SEQUENCE LISTING (130 sequences)

SEQ ID NO: 1 - DNA, 28 bp, Artificial Sequence
  Description of Artificial Sequence synthetic oligonucleotide
cggccatggcccagctgcagactagtgc

SEQ ID NO: 2 - DNA, 35 bp, Artificial Sequence
  Description of Artificial Sequence synthetic oligonucleotide
ggccgcactagtctgcagctgggccatggccggct

SEQ ID NO: 3 - DNA, 45 bp, Artificial Sequence
  Description of Artificial Sequence synthetic oligonucleotide
ctcgcggcccagccggccatggccgaggctgcagtcacccaaagc

SEQ ID NO: 4 - DNA, 32 bp, Artificial Sequence
  Description of Artificial Sequence synthetic oligonucleotide
cttcctcactagtacagtctgctcggccccag

SEQ ID NO: 5 - DNA, 30 bp, Artificial Sequence
  Description of Artificial Sequence synthetic oligonucleotide
gatggcctcgaggagcaggtggagcagctt

SEQ ID NO: 6 - DNA, 33 bp, Artificial Sequence
  Description of Artificial Sequence synthetic oligonucleotide
gactagcccgggacagggaacgtctgaactggg

SEQ ID NO: 7 - DNA, 45 bp, Artificial Sequence
  Description of Artificial Sequence synthetic oligonucleotide
ctcgcggcccagccggccatggccgagcaggtggagcagcttcct

SEQ ID NO: 8 - DNA, 33 bp, Artificial Sequence
  Description of Artificial Sequence synthetic oligonucleotide
ctcgcgctcgaggaggctgcagtcacccaaagc

SEQ ID NO: 9 - DNA, 33 bp, Artificial Sequence
  Description of Artificial Sequence synthetic oligonucleotide
ctcgcgcccgggacagtctgctcggccccaggc

SEQ ID NO: 10 - DNA, 33 bp, Artificial Sequence
  Description of Artificial Sequence synthetic oligonucleotide
ctcgcgactagtacagggaacgtctgaactggg

SEQ ID NO: 11 - DNA, 30 bp, Artificial Sequence
  Description of Artificial Sequence synthetic oligonucleotide
ctcgcgcccggggtctgctcggccccaggc

SEQ ID NO: 12 - DNA, 30 bp, Artificial Sequence
  Description of Artificial Sequence synthetic oligonucleotide
ctcgcgactagtgggaacgtctgaactggg

SEQ ID NO: 13 - DNA, 30 bp, Artificial Sequence
  Description of Artificial Sequence synthetic oligonucleotide
ctcgcgactagtgtctgctcggccccaggc

SEQ ID NO: 14 - DNA, 30 bp, Artificial Sequence
  Description of Artificial Sequence synthetic oligonucleotide
ctcgcgcccggggggaacgtctgaactggg

SEQ ID NO: 15 - DNA, 34 bp, Artificial Sequence
  Description of Artificial Sequence synthetic oligonucleotide
ctcgcgctcgagcgaggctgcagtcacccaaagc

SEQ ID NO: 16 - DNA, 37 bp, Artificial Sequence
  Description of Artificial Sequence synthetic oligonucleotide
ggggggcccggggctgagggtgacgatcccgcaaaag

SEQ ID NO: 17 - DNA, 67 bp, Artificial Sequence
  Description of Artificial Sequence synthetic oligonucleotide
ctagtctggtggcggtggcagcggcggtggtggttccggtggcggcggttctggcggtggcggttcc

SEQ ID NO: 18 - DNA, 67 bp, Artificial Sequence
  Description of Artificial Sequence synthetic oligonucleotide
tcgaggaaccgccaccgccagaaccgccgccaccggaaccaccaccgccgctgccaccgccaccaga

SEQ ID NO: 19 - DNA, 37 bp, Artificial Sequence
  Description of Artificial Sequence synthetic oligonucleotide
gtgctcactagtgtttggctctacagtgagtttggtg

SEQ ID NO: 20 - DNA, 33 bp, Artificial Sequence
  Description of Artificial Sequence synthetic oligonucleotide
gatggctcgagtgagcaggtggagcagcttcct

SEQ ID NO: 21 - DNA, 33 bp, Artificial Sequence
  Description of Artificial Sequence synthetic oligonucleotide
ctagtccccgggtacaactgtgagtctggttcc

SEQ ID NO: 22 - DNA, 33 bp, Artificial Sequence
  Description of Artificial Sequence synthetic oligonucleotide
ctcgagactagttacaactgtgagtctggttcc

SEQ ID NO: 23 - DNA, 33 bp, Artificial Sequence
  Description of Artificial Sequence synthetic oligonucleotide
cggccgaggaagaagagtacatcccgatggatc

SEQ ID NO: 24 - DNA, 40 bp, Artificial Sequence
  Description of Artificial Sequence synthetic oligonucleotide
gggccatccatcgggatgtactcttcttcctcggccggct

SEQ ID NO: 25 - DNA, 36 bp, Artificial Sequence
  Description of Artificial Sequence synthetic oligonucleotide
ccggggaggaagaagagtacatcccgatggattgag

SEQ ID NO: 26 - DNA, 36 bp, Artificial Sequence
  Description of Artificial Sequence synthetic oligonucleotide
aattctcaatccatcgggatgtactcttcttcctcc

SEQ ID NO: 27 - DNA, 15 bp, Artificial Sequence
  Description of Artificial Sequence synthetic oligonucleotide
gcccgggactagtgc

SEQ ID NO: 28 - DNA, 23 bp, Artificial Sequence
  Description of Artificial Sequence synthetic oligonucleotide
ggccgcactagtcccgggctgca

SEQ ID NO: 29 - DNA, 75 bp, Artificial Sequence
  Description of Artificial Sequence synthetic oligonucleotide
ctagtccccgggtcatcaagcggcgccttccatcggcatgtactcttcttcctctacaactgtgagtctggttcc

SEQ ID NO: 30 - DNA, 72 bp, Artificial Sequence
  Description of Artificial Sequence synthetic oligonucleotide
ctagtccccgggtcatcaagcggcgccttccatcggcatgtactcttcttcctcgtctgctcggccccaggc

SEQ ID NO: 31 - DNA, 33 bp, Artificial Sequence; identical to SEQ ID NO: 21
  Description of Artificial Sequence synthetic oligonucleotide

SEQ ID NO: 32 - DNA, 45 bp, Artificial Sequence
  Description of Artificial Sequence synthetic oligonucleotide
ccggggaggaagaagagtacatgccgatggaaggcgccgcttagc

SEQ ID NO: 33 - DNA, 45 bp, Artificial Sequence
  Description of Artificial Sequence synthetic oligonucleotide
cctccttcttctcatgtacggctaccttccgcggcgaatcgggcc

SEQ ID NO: 34 - DNA, 33 bp, Artificial Sequence
  Description of Artificial Sequence synthetic oligonucleotide
gatcagcccggggaggctgcagtcacccaaagc

SEQ ID NO: 35 - DNA, 33 bp, Artificial Sequence
  Description of Artificial Sequence synthetic oligonucleotide
ctagtccccgggacagtctgctcggccccaccg

SEQ ID NO: 36 - DNA, 42 bp, Artificial Sequence
  Description of Artificial Sequence synthetic oligonucleotide
ccggggaggaagaagagtacatgccgatggaaggcgccgctc

SEQ ID NO: 37 - DNA, 42 bp, Artificial Sequence
  Description of Artificial Sequence synthetic oligonucleotide
cctccttcttctcatgtacggctaccttccgcggcgagggcc

SEQ ID NO: 38 - DNA, 32 bp, Artificial Sequence
  Description of Artificial Sequence synthetic oligonucleotide
cgccgctcaccatcaccatcatcactgatgac

SEQ ID NO: 39 - DNA, 34 bp, Artificial Sequence
  Description of Artificial Sequence synthetic oligonucleotide
ggcgagtggtagtggtagtagtgactactgggcc

SEQ ID NO: 40 - DNA, 33 bp, Artificial Sequence
  Description of Artificial Sequence synthetic oligonucleotide
gatcagggcgccgctactgttgaaagttgttta

SEQ ID NO: 41 - DNA, 33 bp, Artificial Sequence
  Description of Artificial Sequence synthetic oligonucleotide
ctgatcggatcctcattaaagccagaatggaaa

SEQ ID NO: 42 - DNA, 45 bp, Artificial Sequence
  Description of Artificial Sequence synthetic oligonucleotide
ccgggctaagcggcgccttccatcggcatgtactcttcttcctcc

SEQ ID NO: 43 - DNA, 42 bp, Artificial Sequence
  Description of Artificial Sequence synthetic oligonucleotide
ccgggagcggcgccttccatcggcatgtactcttcttcctcc

SEQ ID NO: 44 - DNA, 28 bp, Artificial Sequence
  Description of Artificial Sequence synthetic oligonucleotide
ccgggtcatcagtgatgatggtgagcgg

SEQ ID NO: 45 - DNA, 15 bp, Artificial Sequence
  Description of Artificial Sequence synthetic oligonucleotide
gctcgagcttactcc

SEQ ID NO: 46 - DNA, 14 bp, Artificial Sequence
  Description of Artificial Sequence synthetic oligonucleotide
cgctcattaggcgg

SEQ ID NO: 47 - DNA, 15 bp, Artificial Sequence
  Description of Artificial Sequence synthetic oligonucleotide
gtgtacttctgtgcc

SEQ ID NO: 48 - DNA, 15 bp, Artificial Sequence
  Description of Artificial Sequence synthetic oligonucleotide
ctgtgagtctggttc

SEQ ID NO: 49 - DNA, 15 bp, Artificial Sequence
  Description of Artificial Sequence synthetic oligonucleotide
gcaggttctgggttc

SEQ ID NO: 50 - DNA, 17 bp, Artificial Sequence
  Description of Artificial Sequence synthetic oligonucleotide
catttactaacgtctgg

SEQ ID NO: 51 - DNA, 15 bp, Artificial Sequence
  Description of Artificial Sequence synthetic oligonucleotide
cgcctggtactgagc

SEQ ID NO: 52 - DNA, 15 bp, Artificial Sequence
  Description of Artificial Sequence synthetic oligonucleotide
cctcaacctcctgtc

SEQ ID NO: 53 - DNA, 16 bp, Artificial Sequence
  Description of Artificial Sequence synthetic oligonucleotide
cttattccgtggtgtc

SEQ ID NO: 54 - DNA, 15 bp, Artificial Sequence
  Description of Artificial Sequence synthetic oligonucleotide
ccaccctcagaaccg

SEQ ID NO: 55 - DNA, 16 bp, Artificial Sequence
  Description of Artificial Sequence synthetic oligonucleotide
gaatttaccgttccag

SEQ ID NO: 56 - DNA, 16 bp, Artificial Sequence
  Description of Artificial Sequence synthetic oligonucleotide
ctttagcgtcagactg

SEQ ID NO: 57 - DNA, 16 bp, Artificial Sequence
  Description of Artificial Sequence synthetic oligonucleotide
gaaacgcaaagacacc

SEQ ID NO: 58 - DNA, 39 bp, Artificial Sequence
  Description of Artificial Sequence synthetic oligonucleotide
ggggggcccgggctgctgagggtgacgatcccgcaaaag

SEQ ID NO: 59 - DNA, 39 bp, Artificial Sequence
  Description of Artificial Sequence synthetic oligonucleotide
gggggggaattctattagcttgctttcgaggtgaatttc

SEQ ID NO: 60 - DNA, 41 bp, Artificial Sequence
  Description of Artificial Sequence synthetic oligonucleotide
gagcacggcccagccggccatggccgaggctgcagtcaccc

SEQ ID NO: 61 - DNA, 69 bp, Artificial Sequence
  Description of Artificial Sequence synthetic oligonucleotide
gagcacgagactagtagcacgaacaacacggtcgtcgatcggttccggcgggtttggctctacagtgag

SEQ ID NO: 62 - DNA, 86 bp, Artificial Sequence
  Description of Artificial Sequence synthetic oligonucleotide
gatccctcctggacacgcaggatggaaggaagctgctccacctgctcagcacgaacaacacggtcgtcgatcggttccggcggggc

SEQ ID NO: 63 - DNA, 86 bp, Artificial Sequence
  Description of Artificial Sequence synthetic oligonucleotide
catggccccgccggaaccgatcgacgaccgtgttgttcgtgctgagcaggtggagcagcttcctcccatcctgcgtgtccaggagg

SEQ ID NO: 64 - DNA, 39 bp, Artificial Sequence
  Description of Artificial Sequence synthetic oligonucleotide
gaggtggaattctattaagactccttattacgcagtatg

SEQ ID NO: 65 - DNA, 60 bp, Artificial Sequence
  Description of Artificial Sequence synthetic oligonucleotide
gaggaggtggtgactagtagcaggttctggtgggttctggatgtttggctctacagtgag

SEQ ID NO: 66 - DNA, 57 bp, Artificial Sequence
  Description of Artificial Sequence synthetic oligonucleotide
gaggaggtggtgactagaagcaggttctgggttctggatgtttggctctacagtgag

SEQ ID NO: 67 - DNA, 51 bp, Artificial Sequence
  Description of Artificial Sequence synthetic oligonucleotide
gaggtggaattctattagtgatgatggtgatggtgagactccttattacgc

SEQ ID NO: 68 - DNA, 32 bp, Artificial Sequence
  Description of Artificial Sequence synthetic oligonucleotide
gaggtgcccgggactgttgaaagttgtttagc

SEQ ID NO: 69 - DNA, 49 bp, Artificial Sequence
  Description of Artificial Sequence synthetic oligonucleotide
gaggtggaattctattagtgatgatggtgatggtggcttgctttcgagg

SEQ ID NO: 70 - DNA, 31 bp, Artificial Sequence
  Description of Artificial Sequence synthetic oligonucleotide
gaggtggaattctattagcttgctttcgagg

SEQ ID NO: 71 - DNA, 31 bp, Artificial Sequence
  Description of Artificial Sequence synthetic oligonucleotide
gaggtgcccggggctgagggtgacgatcccg

SEQ ID NO: 72 - DNA, 30 bp, Artificial Sequence
  Description of Artificial Sequence synthetic oligonucleotide
aattctcatcagtgatgatggtgatggtgc

SEQ ID NO: 73 - DNA, 30 bp, Artificial Sequence
  Description of Artificial Sequence synthetic oligonucleotide
ccgggcaccatcaccatcatcactgatgag

SEQ ID NO: 74 - DNA, 56 bp, Artificial Sequence
  Description of Artificial Sequence synthetic oligonucleotide
gtggagcccgggttccatcggcatgtactcttcttcctctacaactgtgagtctgg

SEQ ID NO: 75 - DNA, 64 bp, Artificial Sequence
  Description of Artificial Sequence synthetic oligonucleotide
gaggtggaattctcacccgggttccatcggcatgtactcttcttcctcgtctgctcggccccag

SEQ ID NO: 76 - DNA, 61 bp, Artificial Sequence
  Description of Artificial Sequence synthetic oligonucleotide
gaggtgctgcaggttccatcggcatgtactcttcttcctcgtctagacggccccaggcctc

SEQ ID NO: 77 - DNA, 34 bp, Artificial Sequence
  Description of Artificial Sequence synthetic oligonucleotide
gtggagctgcagggtctagacggccccaggcctc

SEQ ID NO: 78 - DNA, 59 bp, Artificial Sequence
  Description of Artificial Sequence synthetic oligonucleotide
gtggagctgcaggtgatccaccccctccagatccaccccctccgtctgctcggccccag

SEQ ID NO: 79 - DNA, 32 bp, Artificial Sequence
  Description of Artificial Sequence synthetic oligonucleotide
gtggagaagctttgccgagcaggtggagcagc

SEQ ID NO: 80 - DNA, 34 bp, Artificial Sequence
  Description of Artificial Sequence synthetic oligonucleotide
gggggggaggtgctggagcgaggcagcagtcacc

SEQ ID NO: 81 - DNA, 35 bp, Artificial Sequence
  Description of Artificial Sequence synthetic oligonucleotide
gagcccactagtttggctctacagtgagtttggtg

SEQ ID NO: 82 - DNA, 65 bp, Artificial Sequence
  Description of Artificial Sequence synthetic oligonucleotide
ctagaccagcaaatctgcacccacagaatccctaggacagctcccaggttcctctgcatggtgga

SEQ ID NO: 83 - DNA, 65 bp, Artificial Sequence
  Description of Artificial Sequence synthetic oligonucleotide
agcttccaccatgcagaggaacctgggagctgtcctagggattctgtgggtgcagatttgctggt

SEQ ID NO: 84 - DNA, 35 bp, Artificial Sequence
  Description of Artificial Sequence synthetic oligonucleotide
gatcggtctagaggtgagcaggtggagcagcttcc

SEQ ID NO: 85 - DNA, 19 bp, Artificial Sequence
  Description of Artificial Sequence synthetic oligonucleotide
gcctggagactcagccatg

SEQ ID NO: 86 - DNA, 21 bp, Artificial Sequence
  Description of Artificial Sequence synthetic oligonucleotide
gaagtacatggctgagtctcc

SEQ ID NO: 87 - DNA, 23 bp, Artificial Sequence
  Description of Artificial Sequence synthetic oligonucleotide
gatgaacgttccagattccatgg

SEQ ID NO: 88 - DNA, 22 bp, Artificial Sequence
  Description of Artificial Sequence synthetic oligonucleotide
cccaaatcaatgtgccgaaaac

SEQ ID NO: 89 - DNA, 53 bp, Artificial Sequence
  Description of Artificial Sequence synthetic oligonucleotide
ctagaacacaggagactggagagcacgaagaagagcctggagcccatggtgga

SEQ ID NO: 90 - DNA, 20 bp, Artificial Sequence
  Description of Artificial Sequence synthetic oligonucleotide
gctctccttgtaggcctgag

SEQ ID NO: 91 - DNA, 20 bp, Artificial Sequence
  Description of Artificial Sequence synthetic oligonucleotide
gtacttctgtgccagcggtg

SEQ ID NO: 92 - DNA, 22 bp, Artificial Sequence
  Description of Artificial Sequence synthetic oligonucleotide
gagcaattatagctactgcctg

SEQ ID NO: 93 - DNA, 20 bp, Artificial Sequence
  Description of Artificial Sequence synthetic oligonucleotide
ggtctggaggccttgtatcc

SEQ ID NO: 94 - DNA, 53 bp, Artificial Sequence
  Description of Artificial Sequence synthetic oligonucleotide
agcttccaccatgggctccaggctcttcttcgtgctctccagtctcctgtgtt

SEQ ID NO: 95 - DNA, 65 bp, Artificial Sequence
  Description of Artificial Sequence synthetic oligonucleotide
tcgaggaaccgccaccgccagaaccgccgccaccggaaccaccaccgccgctgccaccgccacca

SEQ ID NO: 96 - DNA, 65 bp, Artificial Sequence
  Description of Artificial Sequence synthetic oligonucleotide
ctagtggtggcggtggcagcggcggtggtggttccggtggcggcggttctggcggtggcggttcc

SEQ ID NO: 97 - DNA, 8 bp, Artificial Sequence
  Description of Artificial Sequence synthetic oligonucleotide
ccaccatg

SEQ ID NO: 98 - protein, 9 aa, Artificial Sequence
  Description of Artificial Sequence synthetic peptide
EEEEYMPME

SEQ ID NO: 99 - DNA, 63 bp, Artificial Sequence
  Description of Artificial Sequence synthetic oligonucleotide
atgaaatacctgctgccgaccgcagctgctggtctgctgctgctggcggcccagccgatggcc

SEQ ID NO: 100 - protein, 20 aa, Artificial Sequence
  Description of Artificial Sequence synthetic peptide
MKYLLPTAAALLLLAAQPAM

SEQ ID NO: 101 - DNA, 29 bp, Artificial Sequence
  Description of Artificial Sequence synthetic oligonucleotide
gccggccatggccrgtgctgtcrtctctc

SEQ ID NO: 102 - DNA, 29 bp, Artificial Sequence
  Description of Artificial Sequence synthetic oligonucleotide
gccggccatggccgamrccmargtsaccc

SEQ ID NO: 103 - DNA, 29 bp, Artificial Sequence
  Description of Artificial Sequence synthetic oligonucleotide
gccggccatggccgatgtgaaagtaaccc

SEQ ID NO: 104 - DNA, 29 bp, Artificial Sequence
  Description of Artificial Sequence synthetic oligonucleotide
gccggccatggccgamrcwgmcrtytmcc

SEQ ID NO: 105 - DNA, 29 bp, Artificial Sequence
  Description of Artificial Sequence synthetic oligonucleotide
gccggccatggccarkgctggdgtcactc

SEQ ID NO: 106 - DNA, 29 bp, Artificial Sequence
  Description of Artificial Sequence synthetic oligonucleotide
gccggccatggccaatgccggcgtcatgc

SEQ ID NO: 107 - DNA, 29 bp, Artificial Sequence
  Description of Artificial Sequence synthetic oligonucleotide
gccggccatggccgrtrctggagtctccc

SEQ ID NO: 108 - DNA, 29 bp, Artificial Sequence
  Description of Artificial Sequence synthetic oligonucleotide
gccggccatggccgatgctrgartcaccc

SEQ ID NO: 109 - DNA, 29 bp, Artificial Sequence
  Description of Artificial Sequence synthetic oligonucleotide
gccggccatggccgattctggagtcacac

SEQ ID NO: 110 - DNA, 29 bp, Artificial Sequence
  Description of Artificial Sequence synthetic oligonucleotide
gccggccatggccgaygctggwgttatcc

SEQ ID NO: 111 - DNA, 29 bp, Artificial Sequence
  Description of Artificial Sequence synthetic oligonucleotide
gccggccatggccgatgctgryrttaycc

SEQ ID NO: 112 - DNA, 31 bp, Artificial Sequence
  Description of Artificial Sequence synthetic oligonucleotide
gccggccatggccgaagctggagttactcag

SEQ ID NO: 113 - DNA, 36 bp, Artificial Sequence
  Description of Artificial Sequence synthetic oligonucleotide
gccggccatggccgatgctactattcatcaatggcc

SEQ ID NO: 114 - DNA, 31 bp, Artificial Sequence
  Description of Artificial Sequence synthetic oligonucleotide
gctgccaccgccacccaccttgttcaggtcc

SEQ ID NO: 115 - DNA, 30 bp, Artificial Sequence
  Description of Artificial Sequence synthetic oligonucleotide
gctgccaccgccacccacgttttcaggtcc

SEQ ID NO: 116 - DNA, 34 bp, Artificial Sequence
  Description of Artificial Sequence synthetic oligonucleotide
ggaggcggcggttctgctaagagaccacmcagcc

SEQ ID NO: 117 - DNA, 34 bp, Artificial Sequence
  Description of Artificial Sequence synthetic oligonucleotide
ggaggcggcggttctcagaagrtaactcaarcsc

SEQ ID NO: 118 - DNA, 31 bp, Artificial Sequence
  Description of Artificial Sequence synthetic oligonucleotide
ggaggcggcggttctcagtckgtgasccagc

SEQ ID NO: 119 - DNA, 32 bp, Artificial Sequence
  Description of Artificial Sequence synthetic oligonucleotide
ggaggcggcggttctcagagagtgactcagcc

SEQ ID NO: 120 - DNA, 35 bp, Artificial Sequence
  Description of Artificial Sequence synthetic oligonucleotide
ggaggcggcggttctcwgmasgtgraacaargtcc

SEQ ID NO: 121 - DNA, 35 bp, Artificial Sequence
  Description of Artificial Sequence synthetic oligonucleotide
ggaggcggcggttctcagcaagttaagcaaaattc

SEQ ID NO: 122 - DNA, 34 bp, Artificial Sequence
  Description of Artificial Sequence synthetic oligonucleotide
ggaggcggcggttctgagartgtggrgcwgcatc

SEQ ID NO: 123 - DNA, 35 bp, Artificial Sequence
  Description of Artificial Sequence synthetic oligonucleotide
ggaggcggcggttctaakgargtggmgcagrrtyc

SEQ ID NO: 124 - DNA, 32 bp, Artificial Sequence
  Description of Artificial Sequence synthetic oligonucleotide
ggaggcggcggttctcagctgctggagcagag

SEQ ID NO: 125 - DNA, 35 bp, Artificial Sequence
  Description of Artificial Sequence synthetic oligonucleotide
ggaggcggcggttctcaamagrkagaasagratyc

SEQ ID NO: 126 - DNA, 33 bp, Artificial Sequence
  Description of Artificial Sequence synthetic oligonucleotide
ggaggcggcggttctggacaaarcmttgarcag

SEQ ID NO: 127 - DNA, 33 bp, Artificial Sequence
  Description of Artificial Sequence synthetic oligonucleotide
ggaggcggcggttctaaggaccaagtgtttcag

SEQ ID NO: 128 - DNA, 29 bp, Artificial Sequence
  Description of Artificial Sequence synthetic oligonucleotide
ttcatagactagtagggtcagggttctgg

SEQ ID NO: 129 - DNA, 45 bp, Artificial Sequence
  Description of Artificial Sequence synthetic oligonucleotide
ggtggcggtggcagcggcggtggtggttccggaggcggcggttct

SEQ ID NO: 130 - DNA, 47 bp, Artificial Sequence
  Description of Artificial Sequence antisense synthetic oligonucleotide
agaaccgccgcctaccggaaccaccaccgcccgctgccaccgccacc

(19) United States
(12) Patent Application Publication
(10) Pub. No.: US 2007/0116718 A1
(43) Pub. Date: May 24, 2007

(54) Fusion proteins comprising bacteriophage coat protein and asingle-chain T cell receptor

(75) Inventors: Jon A. Weidanz, Amarillo, TX (US); Kimberlyn F. Card, Pembroke Pines, FL (US); Hing C. Wong, Weston, FL (US)

Correspondence Address: EDWARDS & ANGELL, LLP, P.O. BOX 55874, BOSTON, MA, 02205, US

(73) Assignee: Altor BioScience Corporation

(21) Appl. No.: 11/455,225

(22) Filed: Jun. 15, 2006

Related U.S. Application Data
(63) Continuation of application No. 08/813,781, filed on Mar. 7, 1997, now abandoned.

(51) Int. Cl. A61K 39/12; C07H 21/04; C12P 21/04; C07K 14/005; C07K 14/705; C12N 15/86

(52) U.S. Cl. 424/204.1; 536/23.72; 530/350; 435/69.7; 435/456; 435/325

(57) ABSTRACT

The present invention relates to novel fusion proteins comprising a bacteriophage coat protein and a single-chain T cell receptor and uses of such complexes. In one aspect, the invention relates to soluble fusion protein comprising a bacteriophage coat protein covalently linked to a single-chain T cell receptor which comprises a V-alpha chain covalently linked to a V-beta chain by a peptide linker sequence. The soluble fusion proteins of the invention are useful for a variety of applications including: 1) making a bacteriophage library for displaying single-chain T cell receptors for use in screens for identification and isolation of ligands that bind single-chain T cell receptors, and 2) methods for isolating soluble and fully functional single-chain T cell receptors from the fusion proteins.

### RELATED APPLICATIONS

This application is a continuation of U.S. application Ser. No. 08/813,781 filed Mar. 7, 1997, pending. The entire contents which is expressly incorporated herein by reference.

### 1. FIELD OF THE INVENTION

The present invention relates to fusion proteins comprising bacteriophage coat protein and a single-chain T cell receptor, as well as methods of making, and using such fusion proteins. The fusion proteins are useful for a variety of applications including making bacteriophage display libraries to screen binding molecules in vitro.

### 2. BACKGROUND

A T cell response is modulated by antigen binding to a T cell receptor (TCR). One type of TCR is a membrane bound heterodimer consisting of an α and β chain configured to resemble an immunoglobin variable (V) and constant (C) region. The TCR α chain includes a covalently linked V-α and C-α chain, whereas the β chain includes a V-β chain covalently linked to a C-β chain. The V-α and V-β chains form a pocket or cleft that can bind a superantigen or antigen in the context of a major histocompatibility complex (MHC) (known in humans as an HLA complex). See generally Fundamental Immunology 3rd Ed., W. Paul Ed. Rsen Press LTD. New York (1993).

The TCR is believed to play an important role in the development and function of the immune system. For example, the TCR has been reported to mediate cell killing, increase B cell proliferation, and impact the development and severity of various disorders including, cancer, allergies, viral infections and autoimmune disorders. Accordingly, it has been of great interest to develop methods of obtaining TCRs for use in research and medical settings.

In general, TCRs have been difficult to isolate in significant quantities. For example, most TCR preparative methods rely on expression in E. coli where inclusion bodies exemplifying insoluble and improperly folded TCR molecules are often formed. TCRs can be obtained by these methods but only after difficult solubilization and protein re-folding steps. These steps substantially reduce TCR yields and negatively impact TCR stability and functionality.

Other attempts to obtain TCRs have focussed on designing more soluble TCR forms. For example, TCRs have been fused with a variety of polypeptide sequences including immunoglobin constant regions, phosphatidylinositol linkage sequences, and CD3 chains. In many cases, the goal has been to express the TCR on a cell surface to augment protein folding. The TCR fusion proteins are then separated from the cell surface to obtain the TCR.

However, only small amounts of soluble and functional TCR are usually produced by these methods. See e.g., Gregoire, C., et al. (1991) PNAS, 88, 8077; Matsui, K., et al. (1991) Science 254, 1788 (1991); Engel, I., et al. (1992) Science 256, 1318; Lin, A. Y. et al., (1990) Science 249, 677.

Another approach has been to make single-chain T cell receptors (ie. scTCRs) which include a fused V-α and V-β chain. See Novotny, J. et al. PNAS (USA) 88, 8646 (1991); Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); and PCT WO 96/13593;

Ward, E. S. et al., J. Mol. Biol. 224, 885, (1992); and Schlueter, C. J. et al. J. Mol. Biol. 256, 859 (1996).

However, many scTCR isolation methods have been reported to produce insoluble and improperly folded molecules. For example, published PCT application WO 96/18105 discloses methods of expressing scTCRs in E. coli that require difficult protein re-folding and solubilization steps to obtain the scTCR. The methods generally produce scTCRs in low yields. See also Ward, E. S. et al. supra, and Schlueter, C. J. supra.

Several strategies have been developed to improve purification of scTCRs. The strategies include expressing scTCR fusion proteins on a cell surface or in a bacterial periplasmic space. For example, scTCR fusion proteins have been made by fusing scTCRs to a C-β chain and an intracellular signalling domain (see WO 96/18105). However, the scTCR fusion proteins must be cleaved from the cell surface and are often improperly folded, thereby reducing yields. Other methods of making scTCR fusion proteins have included fusing maltose binding protein to focus expression in the periplasmic space (see WO 96/13593). However, the scTCR fusion proteins usually require difficult protein refolding steps to obtain significant amounts of the fusion protein.

It has been of great interest to study interactions of immune system molecules in vitro and in vivo. One way to study the interactions has been to provide immune system molecules in a convenient format. For example, bacteriophage libraries have been used to display antibodies and epitopes for screening binding molecules in vitro. See e.g., Smith, G. P. and Scott, J. K. Methods in Enzym. 217, 228 (1993); Winter, G. et al. Ann. Rev. Immunol. 12, 433 (1994)).

It would thus be desirable to have methods of producing soluble and fully functional scTCR fusion proteins in significant quantities without performing cleaving or protein re-folding steps. It would be further desirable to provide the scTCR fusion proteins in a format which allows in vitro screening of binding molecules.

### SUMMARY OF THF INVENTION

The present invention relates to scTCR fusion proteins that include a scTCR molecule covalently linked (ie. fused) to a bacteriophage coat protein (e.g., gene III or gene VIII protein). The bacteriophage coat protein unexpectedly increases solubility of the scTCR fusion proteins, thereby substantially enhancing yield and functionality thereof. The scTCR fusion proteins are fully soluble and functional, and can be isolated in significant quantities without performing difficult solubilization, cleaving or re-folding steps. Solubility and functionality of the scTCR fusion proteins are further enhanced by providing slow host cell induction conditions, as well as optimizing vector sequences (ribosome binding, leader, and promoter sequences) which affect fusion protein expression. The scTCR fusion proteins can be produced in a variety of formats including bacteriophage display libraries to screen for binding molecules which specifically bind the scTCR fusion proteins.

The fusion proteins of the present invention (sometimes referred to herein as “scTCR fusion proteins” or “soluble fusion proteins”) are unexpectedly soluble. That is, we have found that by fusing an scTCR to a bacteriophage coat protein, a soluble fusion protein can be made without performing difficult solubilization, protein re-folding or cleaving steps. Formation of inclusion bodies in expressing cells is minimal, thereby significantly increasing yields of the soluble scTCR fusion protein. Additionally, the scTCR fusion proteins are unexpectedly functional, ie., the scTCR fusion protein can specifically bind a binding molecule (e.g., antigen) or other ligand in the presence of the fused bacteriophage coat protein.

A scTCR fusion protein of the present invention includes a bacteriophage coat protein or fragment thereof covalently linked to a V-α chain fused to a V-β chain through a flexible peptide linker sequence. Generally, the bacteriophage coat protein is a bacteriophage gene III or gene VIII protein. As used herein “bacteriophage coat protein” includes the full-length coat protein or suitable fragments thereof (see below). The bacteriophage coat protein or suitable fragment is capable of packaging the scTCR into a bacteriophage and displaying the scTCR as a fusion protein component of the bacteriophage coat. Exemplary bacteriophage coat protein fragments and methods for packaging bacteriophage are explained in further detail below.

The scTCR fusion protein of the invention typically also includes one or more fused protein tags (typically one or two) to help purify the scTCR fusion protein from cell components which can accompany it. Alternatively, or in addition, the protein tag can be used to introduce a specific cleavage site in the soluble fusion protein to cleave (ie. separate) the scTCR molecule from the fusion protein. Accordingly, the scTCR fusion proteins of the invention can be used to obtain fully soluble and functional scTCR molecules which are substantially free from the fusion protein.

The scTCR fusion proteins of the present invention provide a number of significant advantages. For example, prior practice generally required prolonged preparative procedures to obtain scTCRs fusion, often requiring solubilization and protein re-folding steps to obtain significant yields thereof. In contrast, the scTCR fusion proteins of the present invention can be readily isolated and purified without performing these steps, thereby substantially increasing solubility, yields, stability and functionality of the fusion proteins. Importantly, analysis of scTCR interactions with antigen presenting cells (APCs) and superantigens will be facilitated by the use of the fusion proteins. Additionally, a wide variety of fusion proteins can be presented for interaction with superantigens or APCs as will be described more fully below.

The fusion proteins of the present invention include scTCRs that are fully soluble and functional. The fusion proteins are thus compatible with a number of expression systems which can be used to test interaction between the fusion proteins and desired ligands.

The present scTCR fusion proteins are useful in a variety of applications. For example, a DNA segment encoding an scTCR fusion protein of the invention can be used to make a bacteriophage display library. In contrast to libraries that express scTCR fragments, the present bacteriophage libraries express full length scTCRs as fusion proteins. Accordingly, use of the present bacteriophage libraries positively impacts analysis of scTCR, particularly scTCR antigen binding pockets. The present bacteriophage libraries are thus useful in screens employing a variety of binding molecules such as antigens, antibodies, small molecules, superantigens and MHC/HLA peptide complexes. Importantly, the present bacteriophage display libraries express fusion proteins with a V-α and a V-β chain, thereby making the fusion proteins more fully representative of TCRs found in vivo.

Additionally, the present bacteriophage libraries maximize formation of specific binding complexes between scTCR fusion proteins and binding molecules, thereby increasing detection of the binding molecules which are rare or weakly binding. The bacteriophage libraries are also amenable to biopanning techniques (e.g., cell panning and immunopanning). The construction of the bacteriophage libraries and use thereof.

The bacteriophage libraries of the present invention can be provided in a kit for use in screening a variety of binding molecules. More specifically, the kit can include one or more bacteriophage libraries made from cells of interest, e.g., T-cells, a suitable host cell strain (e.g., an E. coli strain), and directions for using the kit.

The scTCR fusion proteins of the present invention are expressed as fully soluble and functional polypeptides. When used to construct a bacteriophage display library, the scTCR is typically fused to a gene III or gene VIII protein or suitable fragment thereof to express the scTCR as a component of a bacteriophage coat (capsid). In either format, the scTCR fusion protein can be contacted by a suitable binding molecule to form a specific binding complex. The binding molecule can be detected and purified (if desired) by a variety of standard means, including those disclosed below.

The scTCRs used to construct scTCR fusion proteins can be made from a variety of sources. The sources include publically available TCR DNA sequences as described below or biological sources such as immune cells from a mammal, particularly a primate such as a human.

The present bacteriophage display libraries can be constructed to display scTCR fusion protein muteins. The scTCR fusion protein muteins generally include a bacteriophage coat protein or suitable fragment thereof covalently linked to a scTCR mutein, which scTCR mutein can be selected in accordance with the present invention for increased specific binding affinity for a desired binding molecule. Methods for making scTCR muteins and scTCR fusion protein muteins are disclosed more fully below.

The invention also pertains to a DNA segment comprising a sequence encoding a soluble scTCR fusion protein which includes a bacteriophage coat protein or suitable fragment thereof covalently linked to an scTCR. Generally, the DNA segment is provided as a DNA vector which is capable of expressing the fusion protein in a suitable host cell. The DNA segment includes an operably linked promoter and leader sequence to augment expression of the soluble scTCR fusion protein under host cell culturing conditions described below.

Further contemplated are methods of isolating a soluble fusion protein of the invention which includes introducing (e.g., by transformation or transfection) a DNA vector comprising a sequence which encodes the soluble fusion protein into a suitable host cell and then culturing the host cells in medium under slow induction conditions. Generally, slow induction conditions refer to cell culturing conditions in which an essential nutrient (e.g., an amino acid or phosphate salt) is slowly depleted from the medium over several hours, thereby inducing expression of the sequence encoding the fusion protein. Generally, the DNA vector will be chosen to be inducible during the slow induction conditions. The fusion protein produced thereby can be purified (if desired) to produce substantially pure scTCR fusion protein. As will be disclosed more fully below, the slow induction conditions increase production of the soluble and fully functional scTCR fusion proteins. Alternatively, the above-described method can be adapted to include a DNA vector which encodes a single-chain T cell receptor to express and purify (if desired) a soluble single-chain T cell receptor. The host cell can be a bacterial, insect or mammalian cell.

In some cases it will be desirable for the present scTCR fusion proteins to include one or more suitable protein tags (e.g., 6XHis), to help purify the fusion protein from the cell. Typically, the cell medium or cell extract is contacted with a synthetic matrix which binds the protein tag. Alternatively, or in addition, the scTCR fusion protein can include one or more cleavable protein tags which can be used to separate the scTCR from the fusion protein to produce soluble and fully functional scTCR molecules.

The invention also includes methods of isolating a DNA segment which includes sequence encoding a soluble scTCR fusion protein of the invention. In general, the method includes infecting host cells with a bacteriophage library made in accordance with the present invention, and culturing the cells under conditions (e.g., slow induction conditions) which permit propagation of the bacteriophages in the cells. The infected cells can then be contacted with a desired binding molecule, preferably a detectably-labelled binding molecule, under conditions which permit binding between the binding molecule and at least one of the bacteriophages. Methods for screening bacteriophage libraries are well known in the art and can be found, e.g., in Sambrook et al. infra. The specific binding complex produced thereby is subsequently identified and the bacteriophage isolated and purified (if desired) to isolate the DNA segment included therein which encodes the scTCR fusion protein.

Methods for detectably-labelling binding molecules such as proteins, polypeptides, and nucleic acids are well known in the art and can be found, e.g., in Sambrook et al. infra and Ausubel et al., infra.

Further provided are methods of increasing the specific binding affinity of a desired scTCR. The method generally includes determining a specific binding affinity between the scTCR and a desired binding molecule (e.g., an antigen or other ligand), and infecting suitable host cells with a bacteriophage library that expresses fusion protein muteins. The infected cells are then contacted with the binding molecule, preferably a detectably-labelled binding molecule, to form specific binding complexes, after which, the specific binding complexes are identified. A DNA segment encoding the corresponding fusion protein mutein is subsequently isolated by standard means. If desired, the DNA segment can be subcloned into a suitable DNA vector for propagation in a host cell. The scTCR mutein thus produced can be separated from the fusion protein (if desired) by cleaving a suitable protein tag. Subsequently, the specific binding affinity between the antigen and the scTCR mutein can be determined. The scTCR exhibiting increased specific binding affinity is readily identified as the scTCR mutein with a specific binding affinity greater than the scTCR protein.

An undesired immune response in a mammal may be reduced or eliminated in accordance with the present invention by one or a combination of alternative strategies. In general, the present invention provides treatment methods for reducing or eliminating an immune response in a mammal by providing an effective amount of an scTCR in a pharmaceutically acceptable formulation. Generally, the scTCR will be one which is capable of competing for antigen with one or more TCRs, particularly those TCRs occurring on pathogenic T cells such as those accompanying a viral infection, autoimmune disorder, transplantation rejection or cancer. Particularly, an scTCR having a greater specific binding affinity for antigen than a corresponding TCR (e.g., 2 to 10 fold) can be used to suppress or eliminate the desired immune response. Protein competition assays are well known in the art and can be used to screen desired scTCRs for competition activity.

Additionally, the invention features methods of making antibodies (monoclonal or polyclonal) using standard immunological techniques by using a purified sample of an scTCR or scTCR fusion protein. Typically, the scTCR is obtained from a soluble fusion protein of the invention, thereby increasing yield of the scTCR. The antibodies can also be generated from an immunogenic peptide that comprises one or more epitopes of the scTCR. Such antibodies, particularly monoclonal antibodies, are useful for a variety of applications including detecting pathogenic T cells in a biological sample, such as blood or serum. The antibodies can also be used to deplete or inhibit the activity of targeted T cells in vivo or in vitro particularly by reducing interaction between the targeted T cells and antigen or MHC/HLA peptide complex. In some cases, it may be desirable to covalently attach a suitable cytotoxic or anti-metabolic agent to the antibody as is well known in the art.

The present invention further pertains to methods of inducing an immune response in a mammal, particularly a primate such as a human, by administering to the mammal an effective amount of an scTCR (separated from a scTCR fusion protein of the invention). Isolation of the scTCR is positively impacted by obtaining the scTCR from a soluble and fully functional scTCR fusion protein of the invention. More specifically, the scTCR can be used to immunize the mammal against TCR antigenic structures that occur on the surface of T cells (ie. targeted T cells) and which perform pathogenic or otherwise undesirable functions in an immune response. Such T cells can be identified by a variety of conventional methods in a biological sample. The T cells can then be purified and assayed in vitro for capacity to undergo a proliferative response in accordance with examples provided below. T cells that exhibit a proliferative response can be established as cultured cell lines from which DNA encoding TCRs can be isolated and used to produce corresponding scTCR fusion proteins, including scTCR fusion protein muteins, according to techniques disclosed herein. TCR antigenic structures of interest will typically include clonotypic epitopes, V-α or V-β family-specific epitopes, conformational epitopes, and linear epitopes. Immunization against TCR antigenic structures that occur on the surface of the T cells inhibits the activity of the T cells, thereby reducing or eliminating the pathogenic or undesirable effects of the T cells. The mammal is generally immunized by administering the scTCR (separated from a scTCR fusion protein of the invention) in an effective amount and in a pharmaceutically acceptable mixture, so that the targeted T cells are depleted or eliminated by the host immune system.

Further contemplated are assays for detecting a binding molecule (e.g., an antigen) capable of specifically binding a TCR. Generally, the assays will be in vitro screens in which a bacteriophage library described herein is incubated with the molecule under conditions which permit the molecule to specifically bind at least one of the bacteriophages in the library. The specific binding complex produced between the scTCR fusion protein displayed by the bacteriophage and the binding molecule can be readily detected. Generally, the presence of the specific binding complex is indicative of the binding molecule capable of specifically binding a TCR which corresponds to the scTCR encoded by the bacteriophage. The selected bacteriophage can be readily propagated to isolate the DNA segment encoding the scTCR fusion protein by methods disclosed herein.

The invention further pertains to methods of detecting a binding molecule which is capable of inhibiting specific binding between a an antigen and a TCR. The assays involve in vitro screens in which an scTCR fusion protein of the invention is incubated with binding molecules under conditions which permit the antigen and the fusion protein to form a specific binding complex. In a separate reaction, the fusion protein is incubated with the antigen and the binding molecule under the same or similar incubation conditions. Thereafter, the interaction between the antigen and the fusion protein in the absence and presence of the ligand is evaluated by standard binding assays. The ligand capable of inhibiting specific binding between the antigen and the TCR is one in which there is observed less interaction between the fusion protein and the antigen in the presence of the ligand.

### BRIEF DESCRIPTION OF THE DRAWINGS

FIGS. 1A and 1B are drawings showing the pJRS149 (1A) and pKC12 (1B) vectors.

FIG. 2 is a flow chart outlining the construction of DNA vectors encoding fusion proteins.

FIG. 3 is a drawing illustrating portions of the pKC44, pKC46 and pKC51 DNA vectors.

FIG. 4 is a flow chart outlining the construction of the pKC45, pKC46 (pSUN18) and pKC51 DNA vectors.

FIG. 5 is a drawing illustrating the PEN2 DNA vector.

FIGS. 6A and 6B are drawings showing portions of (6A) pKC61, pKC63, and pKC65, and (6B) pKC60, pKC62 (pSUN19), pKC64, pKC66 and pKC67 DNA vectors.

FIG. 7 is a Western blot comparing synthesis of fusion proteins from the pKC60 and pKC46 DNA vectors.

FIG. 8 is a Western blot showing expression of soluble scTCR fusion proteins in MM294, K91Kan, X11-B, TB-1, and UT-5600 cell lines.

FIG. 9 is a drawing outlining immunoaffinity purification of a scTCR fusion proteins which include bacteriophage gene VIII protein.

FIG. 10 is a Western Blot showing fusion protein expression from DNA vectors pKC51 and pKC46 as well as purified fractions thereof.

FIG. 11 is an SDS-PAGE gel stained with coomassie blue showing purified scTCR fusion protein from the pKC51 vector.

FIG. 12 is a Western blot of purified scTCR fusion protein from the pKC51 vector.

FIG. 13 is a Western blot illustrating immunoprecipitation experiments with scTCR fusion proteins pKC60 and pKC62.

FIG. 14 is a graph showing surface plasma resonance analysis of a fusion protein encoded by pKC51.

FIG. 15 is a drawing schematically showing interaction of anti-TCR (V-β 8.2) (MR5-2), anti-αβTCR (H57-597 antibody), and anti-M13 antibodies by surface plasma resonance analysis.

FIG. 16 is a graph showing surface plasma resonance analysis of binding between the scTCR fusion protein encoded by. pKC60 and SEC3 superantigen.

FIG. 17 is a graph comparing capture of bacteriophages expressing scTCR fusion protein with an anti-HS7 antibody by ELISA. The scTCR fusion protein was expressed from the pcK46 vector.

FIG. 18 is a graph showing titration of bacteriophage expressing an scTCR fusion protein using pKC51 vector by ELISA.

FIG. 19A and 19B are graphs showing inhibition of IL-2 production of gD12 (19A) and D011.10T (19B) hybridoma cells by scTCR fusion protein encoded by the pKC51 vector.

FIG. 20 is a drawing illustrating pKC70, pKC71, and pKC72 bacteriophage vectors.

FIG. 21 (21A-21G) are tables showing DNA oligonucleotide primers used to construct DNA vectors described below. FIG. 21A (SEQ ID NOs.: 1-16), FIG. 21B (SEQ ID NOs: 17-30), FIG. 21C (SEQ ID NOs: 31-45), FIG. 21D (SEQ ID NOs: 46-63), FIG. 21E (SEQ ID NOs: 64-79), FIG. 21F (SEQ ID NOs: 80-95), FIG. 21G (SEQ ID NOs: 97-100).

FIG. 22 is a table showing DNA oligonucleotide primers used to construct V-β chains of scTCR fusion proteins (SEQ ID NOs: 116-131).

FIG. 23 is a table showing DNA oligonucleotide primers used to construct V-α chains of scTCR fusion proteins (SEQ ID NOs: 101-115).

### DETAILED DESCRIPTION OF THE INVENTION

As summarized above, we have made fully soluble and functional scTCR fusion proteins that include a bacteriophage coat protein or suitable fragment thereof covalently linked to an scTCR molecule. The scTCR includes a V-α chain covalently linked to a V-β chain by a single-chain peptide linker sequence. DNA segments and vectors encoding the scTCR fusion proteins are useful for constructing bacteriophage libraries to display the scTCR fusion proteins in a format suitable for detecting binding molecules in vitro.

In general, preparation of the present scTCR fusion proteins can be accomplished by procedures disclosed herein and by recognized recombinant DNA techniques. For example, preparation of plasma DNA, DNA cleavage with restriction enzymes, ligation of DNA, transformation, transfection electroporation or biolistic transfer of a cell host, culturing the cell host, and isolation and purification of the expressed DNA are known techniques. See generally Sambrook et al. in Molecular Cloning: A Laboratory Manual (2d ed. 1989); and Ausubel et al., Current Protocols in Molecular Biology, John Wiley & Sons, New York, 1989; each reference fully incorporated herein by reference.

The scTCR fusion proteins of the present invention are single-chain fusion proteins which include a bacteriophage coat protein or suitable fragment thereof fused to a scTCR.

Typically, the bacteriophage coat protein or fragment will be a gene III or gene VIII protein from a filamentous phage of E. coli. The scTCR includes a V-α chain fused to a V-β chain by a suitable peptide linker. The scTCR will thus correspond to V-α and V-β chains from TCRs in T cells. The T cells can be naturally occurring or can be found in association with disease, e.g., as in immune system pathologies accompanying cancer, autoimmune system disorders, allergies, or viral infections. Generally, the V-α and V-β chains of the scTCR fusion protein are approximately 200 to 400 amino acids in length, preferably approximately 300 to 350 amino acids in length, and will be at least 90% identical, and preferably 100% identical to the V-α and V-β chains of the TCR. By the term “identical” is meant that the amino acids of the V-α or V-β chain are 100% homologous to the corresponding TCR V-α or V-β chains. Specific binding between an scTCR fusion protein of the invention and a binding molecule can be assay by a number of techniques including immunoadsorption and immunoprecipitation.

As mentioned previously, the scTCR can be fused to a bacteriophage coat protein or fragment thereof from a filamentous phage of E. coli. Exemplary filamentous phages are disclosed in Sanbrook et al., supra; and Smith and Scott, supra. As used herein “fragment” means a portion of bacteriophage coat protein that is capable of being packaged into a bacteriophage and displaying a soluble fusion protein of the invention on the bacteriophage surface. In general, a bacteriophage gene VIII fragment will be approximately 40 to 50 amino acids in length, preferably approximately 50 amino acids in length. A bacteriophage gene III fragment will be approximately 200 to 400 amino acids in length, preferably approximately 400 amino acids in length. Methods for determining if a bacteriophage coat protein fragment is suitably packaged into a bacteriophage are known and generally involve plaque-type assays. Additionally, the bacteriophage coat protein fragment will be capable of displaying the scTCR fusion protein on the surface of the bacteriophage as determined by, standard immunological methods such as biopanning assays described below. The bacteriophage coat protein fragments can be made by a variety of methods including mutagenizing DNA vectors described herein (e.g., by site directed mutagenesis or end deletion) to remove portions of DNA encoding the bacteriophage coat protein. The DNA vector can then be used to fuse DNA encoding the fragment to a desired DNA segment encoding a scTCR.

The V-α chain of the scTCR fusion protein is covalently linked to the V-β chain through a suitable peptide linker fused to the C-terminus of the V-α chain and the N-terminus of the V-β chain. The V-β chain can further include a C-β chain fragment fused to the C-terminus of the V-β chain, and the V-α chain can further include a C-α fragment fused to the C-terminus of the V-α chain and the N-terminus of the peptide linker. Generally, the C-β chain fragment will have a length of approximately 50 to 126 amino acids. Importantly, the C-β chain fragment will not include the last cysteine residue at position 127. The C-α chain fragment will have a length of approximately 1 to 21 amino acids, starting from approximately amino acid 1 (isoleucine) to 21 (leucine) of the C-α chain. The C-α chain fragment will not include any cysteine resides. A bacteriophage coat protein or fragment thereof is fused to the C-terminus of the V-β chain or C-β fragment. Alternatively, a protein tag can be fused to the C-terminus of the V-β chain (or C-β chain fragment) and the N-terminus of the bacteriophage coat protein or fragment thereof. The protein tag can also be fused to the C-terminus of the V-β chain (or C-β chain fragment) and the N-terminus of the bacteriophage coat protein or fragment thereof, and a second protein tag (the same or different) can be fused to the C-terminus of the bacteriophage coat protein or fragment thereof. Alternatively, the protein tag can be fused to the C-terminus of the bacteriophage coat protein or fragment thereof, which bacteriophage coat protein or fragment is further fused at the N-terminus to the C-terminus of the V-β chain (or C-β chain fragment).

Further contemplated scTCR fusion proteins include those with a protein tag fused to the N-terminus of the V-α chain. Additionally, the bacteriophage coat protein or fragment thereof can be fused to the C-terminus of the V-α chain (or C-α fragment) which chain is covalently linked to the C-terminus of the V-β (or C-β fragment) through a peptide linker fused to the N-terminus of the V-α chain.

Additional scTCR fusion proteins of the invention include two peptide linker sequences, where the first peptide linker sequence is fused to the C-terminus of the V-α chain and the N-terminus of the V-β chain. The C-terminus of the V-β chain is fused to the N-terminus of a suitable C-β chain fragment. The second peptide linker is thus fused to the C-terminus of the C-β chain fragment and the N-terminus of the bacteriophage coat protein or suitable fragment thereof.

The scTCR molecule included in the fusion protein comprises a peptide linker sequence flexibly employed to provide effective positioning of the V-α and V-β chains. That is, the peptide linker sequence positions the V-α and V-β chains to a pocket which can specifically bind a molecule such as an antigen. Antigen binding to the scTCR fusion protein can be used to modulate T cell activity as determined by assays described below. Exemplary of such assays include in vitro assays involving sequential steps of culturing T cells expressing a TCR to proliferate and contacting the T cells with the scTCR fusion protein (or scTCR obtained therefrom) and then evaluating whether the fusion protein is capable of modulating activity of the T cells.

In any of the above-mentioned scTCR fusion proteins, the V-β chain can be covalently linked to the V-α chain through a suitable peptide linker fused to the C-terminus of the V-β chain and the N-terminus of the V-α chain.

A DNA segment encoding a desired V-α and V-β chain can be obtained from a variety of sources including T cell hybridomas, and T cells such as cytotoxic T cells (CTLs). The CTLs can be naturally-occurring or can be associated with a pathogenic immune system response in a rodent (e.g., mouse, rat, rabbit) or primate (e.g. human or chimpanzee). For example, CTLs can be derived from patients suffering from or suspected of having a viral infection, cancer, an autoimmune disorder, allergy or transplantation rejection response. More particularly, CTLs can be isolated from patients infected with a DNA or RNA virus to make scTCR fusion proteins in accordance with the present invention. For example, scTCR fusion proteins can be prepared using CTLs isolated from HIV-infected patients categorized as long-term non-progressors. See Example 18, below. Other sources of CTLs are antigen-specific CTLs and TILs isolated from patients with established carcinomas and melanomas (see e.g., Cox A. et al. Science (1994) 264: 716; Rosenberg, S. A. et al. N. Eng. J. Med. (1988) 319: 1676; Kawakami, Y. et al., J. Exp. Med. (1994) 180: 347); Kawakami, Y. et al. PNAS (1994) 91:6458). Pathogenic immune responses of particular interest include those accompanying multiple sclerosis, insulin dependent diabetes, rheumatoid arthritis, allergies, cancer (ie. immune responses against tumor associated antigens such as CEA), tissue rejection in a patient undergoing transplant surgery such as organ or skin transplant surgery, or an infectious disease, particularly an infectious disease involving an RNA or DNA virus. Particular viruses of interest include the human immunodeficiency viruses (HIV), cytomeglovirus (CMV), influenza, hepatitis, pox virus, Epstein Barr, adenovirus or polyoma viruses. Alternatively, DNA segments encoding the V-α and V-β chains can be obtained from public databases disclosing TCR DNA sequences such as those disclosed below.

With respect to obtaining V-α and V-β chains from cell sources, several alternative procedures can be used to prepare the DNA segments. More particularly, to prepare α and β chain DNA, mRNA is isolated from those cells demonstrating a desired TCR binding specificity. Such methods generally include use of a Polymerase Chain Reaction ie. (PCR) protocol using first-strand cDNA template made from the mRNA. Standard recombinant techniques can then be employed to make the desired α and β chains. The DNA segment encoding the desired α and β chains is then modified to include a suitable peptide linker sequence and protein tag(s), if desired, afterwhich the DNA is packaged in an appropriate packaging system to produce recombinant bacteriophages displaying the scTCR fusion proteins. DNA segments encoding the scTCR fusion protein can be readily isolated from bacteriophages by conventional recombinant techniques, including subcloning techniques, or PCR amplification methods employing DNA oligonucleotide primers which hybridize to bacteriophage DNA flanking a scTCR fusion protein insert. Generally, the oligonucleotide primers will be between approximately 12 to 50 nucleotides in length preferably approximately 20-25 nucleotides in length. The DNA encoding the fusion protein thus obtained can be used to prepare significant quantities of the soluble fusion protein (milligram quantities per gram cells) in a suitable host cell such as E. coli.

The DNA segment typically includes a leader sequence to provide appropriate cell processing. In general, the leader sequence is fused to at the 5′ end of the sequence encoding the scTCR fusion protein. More specifically, the leader will be covalently linked to the 5′ end of the DNA sequence encoding V-α chain, or in some embodiments, the V-β chain. It will be recognized however that although a specific leader sequence is linked to particular α or β chain in a vector, leader sequences can often be exchanged using recombinant techniques without a detrimental effect on the processing of the fusion protein. Thus in one preferred embodiment, the 5′ end of the V-α chain is covalently linked to the 3′ end of the leader sequence. The leader sequence will be between approximately 12 to 26 amino acid residues in length. A preferred leader sequence is the modified Pel B sequence disclosed below in Example 3.

The DNA segment typically also includes a promoter such as the trp operon promoter, lac promoter, trp-lac promoter, lac^{uvs} or phoA promoter. Preferred promoters are those such as phoA which provide strong, regulated expression during slow induction conditions lasting several hours (e.g., 2 to 10 hours). Under suitable culture conditions, most strong promoters are capable of providing fusion protein at levels up to and exceeding approximately 10% of the total host cell protein.

A bacteriophage library of the present invention is readily made in several steps. For example, a DNA segment encoding a scTCR can be made as outlined previously. The DNA is then ligated into a suitable phage or phage-derived vector (e.g., a phagemid) to fuse the DNA to sequence encoding a bacteriophage coat protein, typically the gene III or gene VIII protein. Alternatively, the fusion can be to a bacteriophage coat protein fragment. The recombinant phage or phage-derived vector thus produced includes the scTCR fused to the bacteriophage coat protein or fragment thereof. In some cases, it will be desirable to further include sequence encoding one or more protein tags as described previously or a stop codon. After infecting a suitable E. coli strain, the fusion protein is displayed in multiple copies (typically dozens to hundreds) on bacteriophage surfaces. A helper phage is provided to augment production of recombinant virions displaying scTCRs of interest. General methods for constructing bacteriophage display libraries using different bacteriophage and E. coli strains have been disclosed. See generally, Smith and Scott supra, Parmley, S. F., and Smith, G. P. (1988) Gene 73, 305.

Particular scTCRs used to make the bacteriophage library include those scTCRs with V-α and/or V-β chains derived from a mammal. Examples include primates, particularly human and chimpanzees, rodents, e.g., immunologically naive mice such as nude mice or mice which include a transgene capable of expressing an HLA-AS antigen complex (Vitiello, A. et al., J. Exp. Med., (1991) 175, 1002). Particular humans of interest include those suffering from or suspected of having cancer, an infectious disease, an autoimmune disorder, or transplantation rejection.

A bacteriophage library according to the present invention can be constructed to display fusion protein muteins. The term “mutein” is used to refer to a fusion protein that includes an average of one amino acid mutation per molecule. More specifically, the amino acid mutation is an amino acid substitution in the scTCR portion of the fusion protein, thereby producing an scTCR mutein. Generally, the scTCR mutein will be constructed to include, on average, one amino acid substitution. Methods for making and analyzing muteins are described more fully below.

The bacteriophage library displaying scTCR fusion protein muteins can be readily made in several steps. For example, a desired scTCR can be made by isolating DNA segments encoding suitable V-α and V-β chains linked by a peptide linker sequence as described previously. The DNA can be ligated together, then modified by introducing mutations, e.g., by site directed mutagenesis, within the desired V-α or V-β regions of the scTCR. Exemplary mutagenesis methods include alanine scanning mutagenesis (see e.g., Nisbet, I. T. et al., (1985) Gene Anal. Tech. 2, 23; Hines, J. C., Gene (1980) 11, 207; see also Sambrook et al. supra). Suitably, the mutations will be amino acid substitutions which affect the binding affinity of the complimentarily determining region (ie. CDR, sometimes referred to as the hypervariable region) of the scTCR. More specifically, the amino acid substitutions will be conservative or non-conservative substitutions which, as used herein, means substitution of one amino acid in the scTCR for another amino acid. In some cases, the substitution will consist of replacement of an amino acid with another amino acid with substantially similar chemical properties (conservative). In other cases, the substitution will consist of replacement of an amino acid with another amino acid having substantially different chemical properties (non-conservative). Accordingly, a tyrosine residue in the scTCR replaced with a phenylalanine residue would represent a conservative amino acid substitution, whereas an alanine residue replaced with a proline residue would represent a non-conservative substitution. It will be understood by those skilled in the art that the mutagenesis may also provide changing the length or amino acid composition of the peptide linker sequence separating the V-α and V-β chains of the scTCR. In most cases however, mutagenesis will be targeted to the V-α or V-β chains and will provide, on average, one amino acid mutation substitution in the V-α or V-β chain of the fusion protein. The extent of mutagenesis can be conveniently assayed by sequencing the DNA of the mutagenized V-α or V-β chain.

Although a less preferred method, a bacteriophage library according to the invention can be also exposed to a chemical mutagen known to introduce amino acid substitutions (e.g., EMS). The scTCR fusion proteins displayed on the surface of the bacteriophages will then thus include fusion protein muteins. If such methods are employed, the amount of chemical mutagen (or exposure to the mutagen) will preferably be adjusted to produce, on average, one amino acid substitution per V-α or V-β chain. As mentioned previously, a bacteriophage library expressing fusion protein muteins (including scTCR muteins) will be particularly useful for the detection of fusion proteins with increased specific binding affinity for a desired antigen.

An scTCR fusion protein according to the invention will usually include an scTCR that corresponds to a TCR. Generally, the T cell expressing the TCR will be found naturally or under pathological conditions in vivo (e.g., T_{S}, T_{C} or T_{H} cells), or the T cell will be a cultured T cell hybridoma (e.g., D10 or B12 cell lines). See Example 1 which follows. As mentioned above, T cells include CTLs isolated from a human suffering from or suspected of suffering from cancer, an infectious disease, an autoimmune disorder or an allergy. The T cells are used to provide DNA encoding TCR sequences (ie. V-α, C-α, V-β and C-β chain sequences); such DNA can be obtained as disclosed herein. Alternatively, the DNA can be obtained by producing oligonucleotide primers homologous to publically available DNA sequences and PCR-amplifying that DNA according to conventional methods. See e.g., Kabat, E. A., et al. (1987) Sequences of Proteins of Immunological Interest, 4th ed. Public Health Service, N.I.H. Washington, D.C. and Chotia, C. et al., (1988) EMBO J. 7:3745.

More particularly, DNA encoding a desired V-α, C-α, V-β or C-β chain sequence can be amplified by PCR or other suitable means such as DNA cloning methods known in the art. In cases where PCR amplification methods are chosen, PCR DNA oligonucleotide primers are selected to amplify pre-determined TCR DNA flanked by oligonucleotide primers (V-α, C-α, V-β or C-β chain sequences). The PCR oligonucleotide primers will typically be between approximately 12 to 50 nucleotides in length, preferably approximately 20 nucleotides in length. The primers can be used to amplify genomic DNA isolated from a desired T cell or DNA provided in a DNA vector which includes DNA encoding the V-α, C-α, V-β or C-β chain sequences. The PCR primers may suitably include restriction sites to add specific restriction enzyme cleavage sites to the PCR product as needed, e.g., to introduce a ligation site. Exemplary primers are provided in the Examples and Drawings which follow. The PCR products produced will include amplified V-α and V-β chain sequences and can be modified to include ribosome binding, leader and promoter sequences as described below for optimal expression of the fusion protein. Suitable primers, PCR conditions and expression vectors are disclosed in the examples and in the Drawings which follow.

The peptide linker sequence effectively positions the V-α and V-β chains of the fusion protein to form an antigen binding pocket. The soluble fusion protein is thus capable of specifically binding antigen (e.g., superantigens, or antigens in the context of MHC/HLA peptide complexes). Importantly, the fusion protein can thus compete with TCRs on the surface of naturally-occurring or pathogenic T cells. By “compete” is meant that the fusion protein is able to bind antigen at a level which is equal to, or preferably exceeds the specific. binding affinity of the corresponding TCR. Generally, the scTCR fusion protein (or scTCR molecule derived therefrom) will be exhibit a binding affinity which is approximately 2 to 10 fold higher than the corresponding TCR. Methods for determining binding affinity are known in the art and are disclosed herein. By the term “corresponding” is meant that TCR (or DNA encoding same) which was used to make the V-α and/or V-β chain of the scTCR.

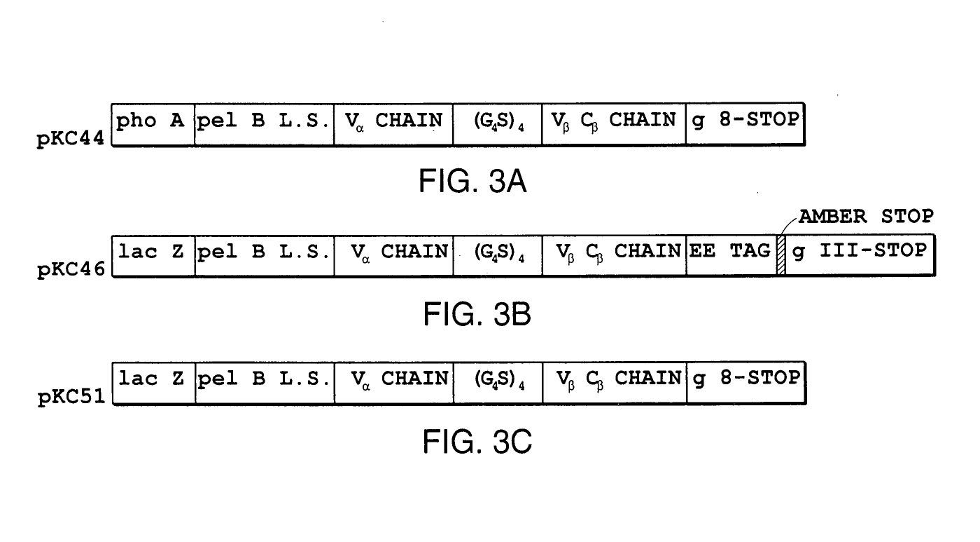

In preferred embodiments of the present invention, the polypeptide linker sequence comprises from about 7 to 20 amino acids, more preferably from about 10 to 20 amino acids, still more preferably from about 12 to 20 amino acids. The linker sequence is typically flexibly disposed in the fusion protein so as to position the V-α and V-β chains in a configuration which optimally binds an antigen. The linker preferably predominantly comprise amino acids with small side chains, such as glycine, alanine and serine, to provide flexibility. Preferably, about 80 or 90 percent or greater of the linker sequence comprises glycine, alanine or serine residues, particularly glycine and serine residues. Preferably, the linker sequence does not contain any proline residues, which could inhibit flexibility. The linker sequence is suitably attached to the C-terminus of the V-α chain and the N-terminus of the V-β chain of a fusion protein. See, e.g., Examples 1, 2 and FIGS. 3, 6A, 6B and 20.

Suitable linker sequences including the (GGGGS)₄ sequence (i.e., Gly Gly Gly Gly Ser)₄ are JA302 (SEQ ID NO: 96) and JA301 (SEQ ID NO: 95). Preferably, a linker sequence is linked between the C-terminal residue of the V-α chain, and the first amino acid of the V-β chain of the scTCR. Different linker sequences can be used including suitable flexible linker designs that have been used successfully to join antibody variable regions together (see M. Whitlow et al., Methods: A Companion to Methods in Enzymology, 2:97-105 (1991)). Such suitable linker sequences can be readily identified empirically. For example, a DNA vector including a DNA segment encoding a fusion protein that includes the linker sequence can be cloned and expressed, and the fusion molecule tested to determine if the molecule is capable of binding antigen, e.g., as in a standard antigen binding assay see e.g., Harlow and Lane, supra. Alternatively, the expressed fusion protein comprising the linker sequence can be tested for capacity to modulate the activity of a T cell as determined by assays disclosed in Examples, 8, 10-12 which follow. Suitable size and sequences of linker sequences also can be determined by conventional computer modeling techniques based on the predicted size and shape of the fusion protein.

Preferably, restriction sites are engineered in the DNA vector so that essentially any nucleotide sequence coding for a V-α chain or V-β chain can be attached to the vector.

Additionally, the DNA vector can be engineered to include one or more restriction sites to add DNA encoding a suitable bacteriophage coat protein (gene VIII or gene III protein) or suitable fragment thereof. Further, DNA encoding one or more protein tags such as myc, EE or 6XHIS can be attached to the DNA vector encoding the fusion protein by conventional methods. See e.g., Manstein, D. S. et al. Gene (1995) 162 (1),129; Grussenmeyer, T. et al. PNAS (USA) (1985) 82, 7952; Tang, E. and Henry, H. L. J. Biol. Chem. (1993) 268 (7), 5069.

More specifically, in one preferred system exemplified in the examples which follow, suitable restriction sites (e.g. XhoI and SpeI sites) are present at the ends of the polypeptide linker sequence between the V-α and V-β chains of the scTCR fusion protein. When fused to an appropriate DNA vector, the restriction sites provide for fusion with the bacteriophage coat protein or fragment thereof.

DNA vectors which include sequence encoding scTCR fusion proteins as disclosed herein generally include (i) an origin of replication functional in E. coli and derived from, e.g., PBR322; (ii) a selectable antibiotic resistance gene, e.g., ampicillin gene; (iii) a transcriptional termination region, e.g., the termination region of the E. coli trp operon; (iv) a transcriptional promoter, e.g., a phoA, tac, tac-lac, lacZ, lac^{uvs}, T7, or T3 promoter; (v) a leader sequence, e.g., a pelB or ompA leader; (vi) a DNA segment encoding the scTCR; (vii) a bacteriophage coat protein such as gene III or gene VIII protein (or a suitable fragment thereof); and (viii) a transcriptional terminator, e.g., the T1T2 sequence from the ribosomal RNA locus of E. coli. In some cases, the DNA vector can further include one or more DNA sequences encoding a protein tag as described previously.

Particular nucleotide sequences can be included in the DNA vector to optimize the expression and stability of the scTCR fusion proteins, particularly under slow induction conditions described herein. For example, the phoA promoter and pelB leader sequences described below are particularly preferred for expressing scTCR fusion proteins during phosphate starvation induction. See Example 4 which follows. A strong translation initiation sequence also can be included in the construct to enhance translation efficiency. For mammalian cell expression, a preferred initiation sequence is the Kozak consensus sequence (CCACCATG) (SEQ ID NO:97).

The leader sequence included in the DNA vector suitably directs expression of the fusion protein to host cell membranes or to the host cell media and includes an effectively positioned restriction site so that a DNA encoding a V-α chain of interest can be conveniently ligated to the construct encoding the fusion protein. Suitably, the restriction site is incorporated into the 3′-end of the leader sequence, sometimes referred to in the art as a junction sequence, e.g. of about 2 to 10 codons in length, and linked to the V-α chain so that the coding region for the V-α chain is typically the first amino acid of the V-α coding region.

For example, one restriction site is the SFiI site, although other cleavage sites can be incorporated before the V-α chain coding region to augment convenient insertion of the V-α chain into the vector construct. As discussed above, use of such a restriction site in combination with a second restriction site, typically positioned at the beginning of the V-α chain, enables rapid and straightforward insertion of sequences coding for a wide variety of V-α chains, or V-α,C-α chains. Preferred leader sequences contain a strong translation initiation site and can sometimes include a cap site at the 3′-end of their mRNA. Exemplary leader sequences include pelB, and OmpA. A particularly preferred leader sequence is pel B as described in Example 4 below.

The term “vector” as used herein means any nucleic acid sequence of interest capable of being incorporated into a host cell resulting in the expression of a nucleic acid of interest such as the scTCR fusion proteins described above vectors can include e.g, linear nucleic acid sequences, plasmids, cosmids, phagmids and extra chromosomal DNA. Specifically, the vector can be recombinant DNA. Also used herein the term “expression,” or “gene expression”, is meant to refer to the production of the protein product of the nucleic acid sequence of interest including transcription of the DNA and translation of the RNA transcription. Typically, a DNA segment encoding an scTCR fusion protein of the invention is inserted into the vector, preferably a DNA vector, to replicate the DNA segment in a suitable host cell.

DNA vectors encoding soluble and fully functional scTCR fusion proteins of the invention were preferably expressed by slowly inducing host cells over an extended time period. Without wishing to be bound to any particular theory, it is believed that slow induction over approximately two to eight hours, preferably approximately four to six hours, stabilizes expression of the scTCR fusion proteins and optimizes intracellular protein folding, particularly when the expression is driven by a strong promoter. For example, a DNA vector was made that included a phoA promoter (strong) operably linked to sequences encoding an scTCR fusion protein. Host cells were then transformed with the DNA vector and phosphate in the host cell media was allowed to deplete from the media over several hours, generally approximately 2 to 10 hours, more typically 4 to 6 hours. It was found that the slow induction conditions combined with use of the strong promoter significantly increased amounts of soluble and fully functional scTCR fusion protein when compared to more rapid induction methods involving use of a chemical inducer and a weak promoter (e.g., IPTG induction of LacZ promoter). See Example 4 which follows. Suitable host cells can be or transformed by a variety of methods including retroviral transfer, calcium-, liposome-, or polybrene mediated transfection, biolistic transfer, or other such techniques known in the art.

As mentioned previously, the scTCR fusion proteins of the present invention can include a one or more protein tags (same or different), including tags which comprise a protease cleavage site. For example, a protein tag can be a polypeptide bearing a charge at physiological pH, such as e.g, 6×HIS in which case a suitable synthetic matrix can be used to purify the fusion protein. More particularly, the synthetic matrix can be a commercially available sepharose matrix, such as e.g. Ni-Sepharose or other such suitable matrixes capable of binding the 6×HIS tag at about pH 6-9. Other suitable tags include EE or myc epitopes which are specifically bound by commercially available monoclonal antibodies. In general, a wide variety of epitopes capable of being specifically bound by an antibody, preferably a commercially available monoclonal antibody are capable of serving as a protein tag. Other suitable synthetic matrices includes those with a bound antibody capable of specifically binding the present scTCR fusion proteins. Exemplary protein tags which include cleavable sites include those with an enterokinase, Factor Xa, snake venom or thrombin cleavage site. See e.g., PCT application WO 96/13593.

A number of strategies can be employed to express soluble fusion proteins of the invention in prokaryotic, eukaryotic, or insect cells. For example, a DNA encoding a fusion protein can be incorporated into a suitable vector by known means such as by use of enzymes to restrict the vector at pre-determined sites to ligate the DNA into the vector. As mentioned previously, the vector will also include sequences encoding a bacteriophage coat protein or a suitable fragment thereof. The vector containing DNA encoding the fusion protein is then introduced into a suitable host for expression of the scTCR fusion protein. Selection of suitable vectors can be empirically based on factors relating to the cloning protocol. For example, the vector should be compatible with, and have the proper replicon for the host cell that is being employed. Further, the vector must be able to accommodate the DNA sequence coding for the fusion protein that is to be expressed. Exemplary vectors include those capable of expressing the fusion proteins in E. coli, particularly in those host strains suitable for construction of a bacteriophage library (see e.g., Smith, G. P. and J. K. Scott, supra). Additional DNA vectors are those capable of expressing the fusion proteins in mammalian cells such as pCDVA3 (available from InVitrogen). See also Sambrook et al., supra and Ausubel et al. supra for additional mammalian vectors.

More particularly, suitable host cells for expressing the fusion proteins of the present invention include cells capable of being readily transformed and exhibiting rapid growth in culture medium. Particularly preferred hosts cells include E. coli, Bacillus subtillus, etc. Other host cells include eukaryotes such as animal cells, yeasts, e.g., S. cerevisiae and insect cells. For the propagation of bacteriophage libraries disclosed herein, E. coli strains such as XL1-B, K91 and K91Kan are preferred. Preferred cells for insect cell expression are those capable of being infected by a baculovirus such as Sf9 cells. In general, conventional culturing conditions are employed in which stably transformed or transfected cell lines are selected e.g., by incorporation of a suitable cell selection marker into the vector (e.g., an antibiotic resistance gene or G418). Cells which express the scTCR fusion protein can be determined by known procedures e.g., ELISA assay using commercially available monoclonal antibodies which specifically bind the V-α or V-β chain as disclosed below.

An expressed scTCR fusion protein can be isolated and purified by known methods including immunoaffinity chromatography, immunoabsorption, immunoprecipitation and the like. Importantly, the preparative procedures will not usually require prolonged refolding steps to obtain significant yields of the fusion protein. In accordance with the protein purification methods described more fully below, yields for most scTCR fusion proteins are in the range of 2 to 20 milligrams per approximately 50 to 100 grams of host cell paste.

In general, to prepare the scTCR fusion proteins, a cell extract or host cell culture medium is centrifuged and the resulting supernatant purified by affinity or immunoaffinity chromatography, e.g. Protein-A or Protein-G affinity chromatography or an immunoaffinity protocol comprising use of an antibody that specifically binds the expressed scTCR fusion molecule. Examples of such an antibody are commercially available monoclonal antibodies capable of specifically binding the V-α chain or V-β chain of the scTCR. Exemplary of such antibodies include H57, MR5-2, and F23.1 obtainable from Pharmagen. Affinity purification of proteins using a monoclonal antibody are generally known and disclosed, e.g., see Harlow and Lane in, Antibodies: A Laboratory Manual (1988).

The fusion proteins of the present invention are provided in a soluble and fully functional form. That is, a form capable of being stably secreted into culture medium. More particularly, the scTCR fusion protein is provided in a form which is stable under substantially physiological conditions in the substantial or complete absence of a chaotropic agent such as a detergent or the like. Thus, the present fusion proteins will not generally include regions rich in hydrophobic amino acids such as those amino acids found in a TCR transmembrane domain. However, in some cases, suitable portions thereof may be included provided that the scTCR fusion protein remains fully soluble. That is, expression of the fusion protein will not lead to formation of significant quantites of inclusion bodies in suitable host cells. Inclusion bodies are readily detectable by microscopy or conventional biochemical techniques.

The scTCR fusion proteins of the present invention can be prepared as discussed above, as well as the examples which follow. Generally, DNA coding for a desired V-α or V-β chain can be obtained from a suitable source such as a T cell, T cell hybridoma line, or publically available V-α and V-β chain sequence as described previously. The DNA can be amplified by PCR, cloning or other suitable means. For example, DNA encoding a desired V-α chain can be cloned into a suitable vector, followed by cloning of DNA encoding a desired V-β chain and a suitable single chain linker sequence to produce a desired scTCR. As disclosed previously, in some cases the scTCR will include a DNA encoding a C-α and/or C-β chain fragment. The scTCR molecule construct is then further fused to a bacteriophage coat protein or suitable fragment thereof (e.g., gene III or gene VIII protein), by ligating DNA encoding the scTCR to a suitably cleaved DNA vector, which vector includes DNA sequence encoding the bacteriophage protein or fragment and other sequences necessary for bacteriophage function. Exemplary DNA vectors which include sequences encoding bacteriophage gene III or gene VIII proteins are disclosed below. As described previously, DNA encoding one or more protein tags can be added to the fusion protein as needed, preferably by selecting a DNA vector which already includes the desired protein tags. The ligated DNA vector is then expressed in a suitable host and the fusion protein harvested and purified if desired.

The scTCR fusion proteins of the present invention are generally encoded by a DNA construct configured to include covalently linked in sequence: promoter/leader sequence/V-β chain/single chain linker sequence/V-β chain; promoter/leader sequence/V-α chain/single chain linker sequence/V-β chain, C-β chain fragment; promoter/leader sequence/V-α chain, C-α chain fragment/single chain linker sequence/V-β chain; or promoter/leader sequence/V-α chain, C-α chain fragment/single chain linker sequence/V-β chain, C-β chain fragment. The DNA vectors are suitably introduced into bacterial cell, baculoviral-insect cell or mammalian cell, including those specific expression systems disclosed herein, for expression of the fusion protein and purification if desired.

Molecular weights of scTCR fusion proteins of the present invention will vary depending on a number of factors including the fused bacteriophage coat protein or fragment chosen, or whether one or more protein tags are employed. In general, a soluble and fully functional fusion protein will have a molecular weight of greater than approximately 45 kDA, in which the V-α and V-β chains therein will have a molecular weight of greater than about 20 kDA, more typically between about 21 to about 26 kDa. Typically, a fusion protein of the present invention will have a molecular weight of about 48 to about 50 kDa. All of the above mentioned molecular weights are determined by conventional molecular sizing experiments such as SDS-PAGE gel electrophesis. See generally Harlow and Lane, supra; Ausubel et al, supra.

Polyvalent scTCR fusion proteins accordingly to the invention are also useful for a number of applications. For example, it is believed that increasing valency of an scTCR can often enhance function. The polyvalent fusion proteins can be made by covalently linking between one and four fusion proteins (the same or different) by using e.g., standard biotin-streptavidin labelling techniques, or by conjugation to suitable solid supports such as latex beads. Chemically cross-linked fusion proteins (for example cross-linked to dendrimers) are also suitable polyvalent species. For example, the fusion protein can be modified by including sequences encoding amino acid residues with chemically reactive side chains such as Cys or His. Such amino acids with chemically reactive side chains may be positioned in a variety of positions in the fusion protein, preferably distal to the antigen binding domain of the scTCR. For example, the C-terminus of a C-β chain fragment of an fusion protein can be covalently linked to a protein purification tag or other fused protein which includes such a reactive amino acid(s). Suitable side chains can be included to chemically link two or more fusion proteins to a suitable dendrimer particle to give a multivalent molecule. Dendrimers are synthetic chemical polymers that can have any one of a number of different functional groups of their surface (D. Tomalia, Aldrichimica Acta, 26:91:101 (1993)). Exemplary dendrimers for use in accordance with the present invention include e.g. E9 starburst polyamine dendrimer and E9 comburst polyamine dendrimer, which can link cysteine residues. Examples of polyvalent fusion proteins can be found in Example 9 which follows.

It also may be desirable to construct DNA vectors encoding a fusion protein of the invention which further includes other agents, particularly a T cell co-stimulatory factor such as those of the B-7 gene family (e.g., B7-1 or B7-2 ) to boost activity of cells including the fusion proteins.

The fusion proteins of the present invention can be used to detect and characterize recombinant peptide antigens, e.g., superantigens or antigens in the context of an MHC or HLA molecule. For example, the methods of the invention can be used to map an uncharacterized epitope for T cells as follows: sequences encoding either a library of random peptides or selected peptides can be provided in a peptide library. The library is then screened with the fusion protein, preferably a detectably-labelled fusion protein. Peptides specifically bound by the fusion protein are then suitably amplified. Sequence analysis of the peptide will identify sequences bound by the fusion protein. Additionally, the cloned peptide sequence can be tested for binding to the T cells expressing a TCR corresponding to the V-α and V-β chains of the fusion protein. Any one of several random peptide libraries can be suitably employed. See e.g., J. Scott et al., Science (1990) 249:386; J. Devlin et al., Science, (1990) 249:404; S. Cwirla et al., PNAS (USA), (1990); 87:6378; J. Hammer et al., J. Exp. Med. (1992) 176:1007; Rhode, P. R. et al. J. Immunol. (1996) 157: 4885; and D. O'Sullivan et al., J. Immunolo., (1991) 147:2663.

Highly useful single-chain class I and class II MHC/peptide complexes (ie. “scMHC complexes”) were disclosed in published PCT Application No. PCT/US95/09816, as well as pending U.S. patent application Ser. Nos. 08/382,454, filed Feb. 1, 1995, and 08/596,387 filed on Jan. 31, 1996. The said published PCT application No. PCT/US95/09816 and pending U.S. application Ser. Nos. 08/382,454 and 08/596,587 also disclose highly useful in vitro and in vivo T cell binding assays which can be used to test the function of the scTCR fusion proteins disclosed herein.

More particularly, the said published PCT application No. PCT/US95/09816 and pending U.S. application Ser. Nos. 08/382,454 and 08/596,587 disclose single-chain MHC class II IA^{d} complexes (ie. “scIa^{d} molecules”) that include a presenting peptide derived from amino acids 323-339 of oval bumin (ie. OVA) or a presenting peptide derived from amino acids 246-261 of the HSV-1 gD peptide (ie. gD12). As disclosed in said pending U.S. application Ser. No. 08/596,587, the OVA and gD12 peptides can be provided in a fused or non-covalently bound to the scIA^{d} complex. The said published PCT application No. PCT/US95/09816 and pending U.S. application Ser. Nos. 08/382,454 and 08/596,587 are each fully incorporated herein by reference.

The ability of a scTCR fusion protein of the present invention to modulate activity of a T cell (ie. reduce or eliminate T cell activity such as proliferation) can be readily determined by in vitro assays. Exemplary in vitro assays and materials for performing the assays have been disclosed previously in said published PCT Application No. published PCT/US95/09816, as well as said pending U.S. patent application Ser. Nos. 08/382,454 and 08/596,387.

In general, suitable T cells for the assays are provided by transformed T cell lines such as T cell hybridomas or T cells isolated from a mammal, e.g., a primate such as from a human or from a rodent such as a mouse, rat or rabbit. Other suitable T cells include: 1) T cell hybridomas which are publicly available or can be prepared by known methods, 2) T helper cells, and 3) T cytotoxic cells, preferably cytotoxic CD8⁺ cells. T cells can be isolated from a mammal by known methods. See, for example, R. Shimonkevitz et al., J. Exp. Med., (1983) 158:303.

As disclosed in said published PCT Application No. US95/09816, as well as said pending U.S. patent application Ser. Nos. 08/382,454 and 08/596,387, an in vitro assay can be performed to determine if a molecule is capable of modulating T cell activity. More particularly, such an assay can be readily adapted for use with the scTCR fusion proteins of the present invention. Generally, the assay is generally conducted as follows, by the sequential steps 1-4 below. T cells suitably express a marker that can be assayed and that indicates T cell activation, or modulation of T cell activity after activation. Thus, e.g., as disclosed in Example 8 below, the murine T cell hybridoma DO11.10 expressing interleukin-2 (IL-2) upon activation can be employed. IL-2 concentrations can be measured to determine if a particular scTCR is capable of modulating activity of this T cell hybridoma. One general example of such a suitable assay is conducted by the following sequential steps:

1. T cell hybridomas or T cells which include a TCR corresponding to an scTCR fusion protein of the invention are obtained. Exemplary T cells are a human CTLs as discussed previously.

2. The T cell hybridomas or T cells are then cultured under conditions that allow proliferation.

3. The proliferating T cell hybridomas or T cells are then contacted with the scTCR fusion protein.

4. The T cell hybridomas or T cells are contacted with an antigen capable of specifically binding the TCR (and scTCR fusion protein) and activating the T cell hybridoma or T cells. Exemplary antigens include superantigens, an sc-MHC class I or II complex bearing a presenting peptide as disclosed above, or a suitable APC.

5. The T cell hybridomas or T cells are contacted with a suitable co-stimulatory factor to provide signals necessary for activation. The T cell hybridomas or T cells are subsequently assayed for a marker, e.g. IL-2 production is measured. A decrease in IL-2 production, e.g., a 40 percent or greater decrease in IL-2 production after a period of 24 hrs., indicates the scTCR fusion protein modulates the activity of the T cells and can suppress an immune response.

Example 8 which follows exemplifies such an assay.

As disclosed previously in said published PCT Application No. US95/09816 and in said pending U.S. patent application Ser. Nos. 08/382,454 and 08/596,387, the T cells employed in the assays are usually incubated under conditions suitable for proliferation. For example, a DO11.10 T cell hybridoma is suitably incubated at about 37° C. and 5% CO₂ in complete culture medium (RPMI 1640 supplemented with 10% FBS, penicillin/streptomycin, L-glutamine and 5×10−5 M 2-mercaptoethanol). Serial dilutions of an fusion protein can be added to the T cell culture medium in concentrations typically in the range of from 10⁻¹² to 10⁻⁶ M. T cell activation signals are preferably provided by antigen presenting cells that have been loaded with the appropriate antigen. It is believed that use of antigen dose and APC numbers giving slightly submaximal T cell activation is preferred to detect inhibition of T cell responses with fusion proteins. A decrease in production of IL-2 following contact with the scTCR indicates the fusion protein modulates activity of the T cells.

As disclosed previously in said published PCT Application No. published PCT/US95/09816 and in said pending U.S. patent application Ser. Nos. 08/382,454 and 08/596,387, rather than measurement of an expressed protein such as IL-2, modulation of T cell activation can be suitably determined by changes in antigen-dependent T cell proliferation as measured by radiolabelling techniques as are recognized in the art. For example, a detectably-labeled (e.g., tritiated) nucleotide may be introduced into an assay culture medium. Incorporation of such a tagged nucleotide into DNA serves as a measure of T cell proliferation. This assay is not suitable for T cells that do not require antigen presentation for growth, e.g., T cell hybridomas. It is suitable for measurement of modulation of T cell activation for untransformed T cells isolated from mammals. A decrease in the level of T cell proliferation following contact with the fusion protein indicates that the molecule modulates activity of the T cells and can suppress immune response, e.g., see Example 8, 10 which follows. The in vitro T cell proliferation assay is preferred for measuring the effects of fusion proteins on antigen-specific changes in T cell clonal expansion in vivo. Such an assay is specifically described in Example 11 which follows.

As described in the examples below, measurement of IL-2 production or T cell proliferation can be employed to determine if the scTCR fusion protein is capable of modifying T cell activation. For example, a decrease in IL-2 production of APC-stimulated T cells after contact by the fusion protein indicates that the fusion molecule modulates activity of the T cells and can suppress a T cell mediated immune response.

In vivo assays also may be suitably employed to determine the ability of a fusion protein to modulate the activity of T cells, including the ability to inhibit or inactivate T cell development. For example, the scTCR fusion protein can be assayed for its ability to inhibit immunoglobulin class switching (i.e. IgM to IgG). See e.g., P. Linsley et al., Science, (1992) 257:792-795.

Diagnostic methods using fusion proteins are also provided including in vivo diagnostic imaging and HLA typing (see, e.g., A. K. Abbas, Cellular and Molecular Immunology, page 328 (W.B. Saunders Co. 1991). For in vivo imaging applications, the fusion protein includes a radioactive label (e.g., ¹²⁵I, ³²P, ⁹⁹Tc) or other detectable tag which can be administered to a mammal and the subject scanned by known procedures for binding of the scTCR. Such an analysis of the mammal could aid in the diagnosis and treatment of a number of disorders including e.g. undesired expression of APCs accompanying immune system disorders.

Assays also may be employed to evaluate use of the scTCR fusion protein (or preferably scTCR obtained from the fusion protein) for treatment of an autoimmune disorder.

For example, as disclosed in said published PCT Application No. US95/09816, as well as said pending U.S. patent application Ser. Nos. 08/382,454 and 08/596,387, experimental allergic encephalomyelitis (EAE) is an autoimmune disease in mice and a recognized model for multiple sclerosis. In one exemplary assay, a mouse strain can be treated to develop EAE and then a suitable scTCR fusion protein (or scTCR obtained from the fusion protein) can be administered. The animal can then be evaluated to determine if EAE development is inhibited or prevented after administration of the fusion protein or scTCR. Such an assay is specifically described in Example 12 which follows.

The ability of a scTCR fusion protein of the invention to include an immune response, including vaccination against a targeted disorder as disclosed previously, may be readily determined by an in vivo assay. For example, the fusion protein (or preferably the scTCR obtained from the fusion protein), can be administered to a mammal such as a mouse, blood samples obtained from the mammal at the time of initial administration and several times periodically thereafter (e.g. at 2, 5 and 8 weeks after administration of the scTCR fusion protein). Serum is collected from the blood samples and assayed for the presence of antibodies raised by the immunization. Antibody concentrations may be determined in accordance with standard immunological techniques.

The present invention also provides a method of administering a DNA segment encoding a fusion protein to express the fusion protein within cells of the mammal, particularly a primate such as a human. Preferably, DNA carrying the coding regions of the fusion protein, suitably under the control of an appropriate promoter such as the CMV promoter, is injected directly into skeletal muscle of the subject according to known methods.

Methods for administration of plasmid DNA, uptake of that DNA by cells of the administered subject and expression of protein has been reported (see J. Ulmer et al. Science, (1993) 259:1745-1749).

The scTCR fusion proteins of the present invention have a number of therapeutic applications. For example, the fusion proteins (or preferably the scTCR obtained from the fusion protein) can be administered to reduce or eliminate an immune response in a mammal, e.g., to treat a mammal including a human that suffers from or is susceptible to cancer, an infectious disease, allergy or an autoimmune disorder such as e.g. multiple sclerosis, insulin-dependent diabetes mellitus, rheumatoid arthritis and the like. Administration can be via any suitable means such as direct administration of DNA encoding the fusion protein. Also suitable for treatment are those subjects suffering or likely to suffer from an undesired immune response e.g. patients undergoing some type of transplant surgery such as transplant of heart, kidney, skin or other organs. In situations involving transplant rejection, a treatment protocol may suitably be commenced in advance of the surgical procedure.

A number of distinct approaches can be employed to reduce or eliminate an immune response of a mammal in accordance with the invention. For example, one treatment method for reduction of an undesired immune response provides for the administration of an effective amount of a desired scTCR fusion protein (or preferably the scTCR derived therefrom) to reduce or eliminate interaction between pathogenic T cells and an antigen. Accordingly, T cell mediated immune responses such as T cell proliferation, differentiation, activation or B lymphocyte stimulation can be selectively controlled. Preferably, the fusion protein exhibits at least the same or preferably increased specific binding affinity for antigen as the pathogenic T cells mediating the undesired immune response. Particularly preferred in this respect are scTCR muteins described previously which demonstrate increased specific binding affinity for ligand.

Administration of scTCRs or antibody to a TCR prepared in accordance with the methods disclosed herein can be administered to a mammal by injection, e.g., intraperitoneal or intravenous injection. An fusion protein, least those molecules used in therapeutic applications, are preferably produced from mammalian cells or other suitable cells and purified prior to use so it is essentially or completely free of pyrogens. The optimal dose for a given therapeutic applications can be determined by conventional means and will generally vary depending on a number of factors including the route of administration, the patient's weight, general health, sex, and other such factors recognized by the art-skilled.

Administration can be in a single dose, or a series of doses separated by intervals of days or weeks. The term “single dose” as used herein can be a solitary dose, and can also be a sustained release dose. The subject can be a mammal (e.g,. a human or livestock such as cattle and pets such as dogs and cats) and include treatment as a pharmaceutical composition which comprises the scTCR or the antibody. Such pharmaceutical compositions of the invention are prepared and used in accordance with procedures known in the art. For example, formulations containing a therapeutically effective amount of an the fusion protein may be presented in unit-dose or multi-dose containers, e.g., sealed ampules and vials, and may be stored in a freeze dried (lyophilized) condition requiring only the addition of the sterile liquid carrier, e.g. water injections, immediately prior use. Liposome formulations also may be preferred for many applications. Other compositions for parenteral administration also will be suitable and include aqueous and non-aqueous sterile injection solutions which may contain anti-oxidants, buffers, bacteriostat and solutes which render the formulation isotonic with the blood of the intended recipient; and aqueous and non-aqueous sterile suspensions which may include suspending agents and thickening agents.

Methods of the invention which include reducing or eliminating T cell responses also may be used in combination with known immunosuppressive, anti-viral, anti-cancer or anti-inflammatory agents to provide a more effective treatment of a T cell mediated disorder. For example, the fusion proteins can be used in combination with conventional immunosuppressive drugs such as cyclosporin or anti-inflammatory agents such as corticosteroids and non-steroidal drugs for the treatment of autoimmune disorders and allergies.

As mentioned previously, the scTCR fusion proteins (or scTCR molecules separated therefrom) can be used to produce antibodies by techniques generally known in the art, and are typically generated to a purified sample of the fusion protein. In most cases, the scTCR fusion protein chosen for raising the antibodies will be first cleaved as described previously to remove the bacteriophage coat protein or fragment thereof. The scTCR thus obtain is used as an immunogen. The antibodies also can be generated from an immunogenic peptide that comprises one or more epitopes of an scTCR of interest.

More particularly, antibodies can be prepared by immunizing a mammal with a purified sample of the scTCR fusion protein, an scTCR molecule separated from the fusion protein, or an immunogenic peptide as discussed above, alone or complexed with a suitable carrier. Preferably, the scTCR molecule will be used as immunogen. Suitable mammals include typical laboratory animals such as sheep, goats, rabbits, guinea pigs, rats and mice. Rats and mice, especially mice, are preferred for obtaining monoclonal antibodies. The antigen can be administered to the mammal by any of a number of suitable routes such as subcutaneous, intraperitoneal, intravenous, intramuscular or intracutaneous injection. The optimal immunizing interval, immunizing dose, etc. can vary within relatively wide ranges and can be determined empirically based on this disclosure. Typical procedures involve injection of the antigen several times over a number of weeks. Antibodies are collected from serum of the immunized animal by standard techniques and screened to find antibodies specific for the scTCR. Of particular interest are those antibodies which specifically bind the V-α or V-β chain, particularly the hypervariable region therein, including those antibodies which recognize linear or conformational epitopes thereon. Monoclonal antibodies can be produced in cells which produce antibodies and those cells used to generate monoclonal antibodies by using standard fusion techniques for forming hybridoma cells. See G. Kohler, et al., Nature, (1975) 256:456. Typically, this involves fusing an antibody producing cell with an immortal cell line such as a myeloma cell to produce the hybrid cell. Alternatively, monoclonal antibodies can be produced from cells by the method of Huse, et al., Science, (1989) 256:1275.

One suitable protocol provides for intraperitoneal immunization of a mouse with a composition comprising purified scTCR complex conducted over a period of about two to seven months. Spleen cells then can be removed from the immunized mouse. Sera from the immunized mouse is assayed for titers of antibodies specific for the scTCR prior to excision of spleen cells. The excised mouse spleen cells are then fused to an appropriate homogenic or heterogenic (preferably homogenic) lymphoid cell line having a marker such as hypoxanthine-guanine phosphoribosyltransferase deficiency (HGPRT) or thymidine kinase deficiency (TK⁻). Preferably a myeloma cell is employed as the lymphoid cell line. Myeloma cells and spleen cells are mixed together, e.g. at a ratio of about 1 to 4 myeloma cells to spleen cells. The cells can be fused by the polyethylene glycol (PEG) method. See G. Kohler, et al., Nature, supra. The thus cloned hybridoma is grown in a culture medium, e.g.

RPMI-1640. See G. E. More, et al., Journal of American Medical Association, (1967) 199:549. Hybridomas, grown after the fusion procedure, are screened such as by radioimmunoassay or enzyme immunoassay for secretion of antibodies that bind specifically to the purified scTCR. An ELISA can be used to screen antibody containing sera according to conventional methods. Hybridomas that show positive results upon such screening can be expanded and cloned by limiting dilution method. Further screens are preferably performed to select antibodies that can bind to scTCR in solution as well as in a biological sample. The isolated antibodies can be further purified by any suitable immunological technique including affinity chromatography.

For some applications, it may be desirable to produce chimeric antibody derivatives which specifically bind scTCRs, e.g. antibody molecules that combine a non-human animal variable region and a human constant region, to thereby render the antibodies less immunogenic in a human subject than the corresponding non-chimeric antibody. A variety of types of such chimeric antibodies can be prepared, including e.g. by producing human variable region chimeras, in which parts of the variable regions, especially conserved regions of the antigen-binding domain, are of human origin and only the hypervariable regions are of non-human origin. See also discussions of humanized chimeric antibodies and methods of producing same in S. L. Morrison, Science, (1985) 229:1202; Oi et al., BioTechniques, (1986) 4:214; Teng et al., Proc. Natl. Acad. Sci. U.S.A., (1983) 80:7308-7312; Kozbor et al., Immunology Today, (1983) 4:7279; Olsson et al., Meth. Enzymol., (1982) 9:3-16.

The term antibody as used herein generally refers to whole immunoglobulin as well immunologically active fragments which bind the scTCR. The immunoglobins and immunologically active fragments thereof include an antibody combining site (i.e., peritope capable of specifically binding the fusion protein). Exemplary antibody fragments include, for example, Fab, F(v), Fab′, F(ab′)₂ fragments, “half molecules” derived by reducing the disulfide bonds of immunoglobulins, single chain immunoglobulins, or other suitable antigen binding fragments (see e.g., Bird et al., Science, (1988) 242; Huston et al., PNAS, (USA), (1988) 85:5879; Webber et al., Mol. Immunol., (1995) 32:249. The antibody or immunologically active fragment thereof may be of animal (e.g., a rodent such as a mouse or a rat), or chimeric form (see Morrison et al., PNAS, (1984) 81:6851; Jones et al., Nature, (1986) 321.

By the term, “specific binding” or a similar term is meant a molecule disclosed herein which binds another molecule, thereby forming a specific binding pair. However, the molecule does not recognize or bind to other molecules as determined by, e.g., Western blotting ELISA, RIA, mobility shift assay, enzyme-immuno assay, competitive assays, saturation assays or other protein binding assays know in the art. See generally, Ausubel, et al supra; Sambrook, et al, supra; Harlow and Lane, supra and references cited therein for examples of methods for detecting specific binding between molecules.

All documents mentioned herein are fully incorporated herein by reference in their entirety.

The following non-limiting examples are illustrative of the present invention.

### EXAMPLE 1

### Construction of Soluble scTCR Fusion Proteins Protein

The DNA sequence of the murine DO11.10 cell TCR has been reported (Kappler, J. et al. PNAS (1994) 91 8462). The TCR recognizes and binds a chicken ovalbumin peptide spanning amino acids 323-339 (ie. OVA) in the context of an I-A^{d} MHC class II molecule. DNA encoding the TCR was prepared generally along the lines of the method disclosed in Kappler and Marrack, supra.

Briefly, mRNA from 1×10⁶ DO11.10 cells was isolated using oligo-dT coated magnetic beads in accordance with the manufacturer's instructions (Dynal). TCR α chain cDNA was made by incubating a mixture containing the C-α specific “back” primer, KC113 (SEQ ID No. 6) along with the DO11.10 mRNA. Subsequently, standard amounts of nucleotides and reverse transcriptase were added to the mixture to form cDNA. The β chain cDNA was made in a similar manner with the exception that the “back” primer KC111 (SEQ ID NO.4) was used instead of the KC113 primer. Alpha chain cDNA was used as a template with primers KC112 (SEQ ID NO. 5) and KC113 in a PCR reaction to amplify a 650 bp 5′ XhoI-3′XmaI α chain fragment. A 750 bp β chain fragment was PCR-amplified using primers KC111 and KC110 (SEQ ID NO. 3) containing SfiI and SpeI sites at the 5′ and 3′ ends respectively.

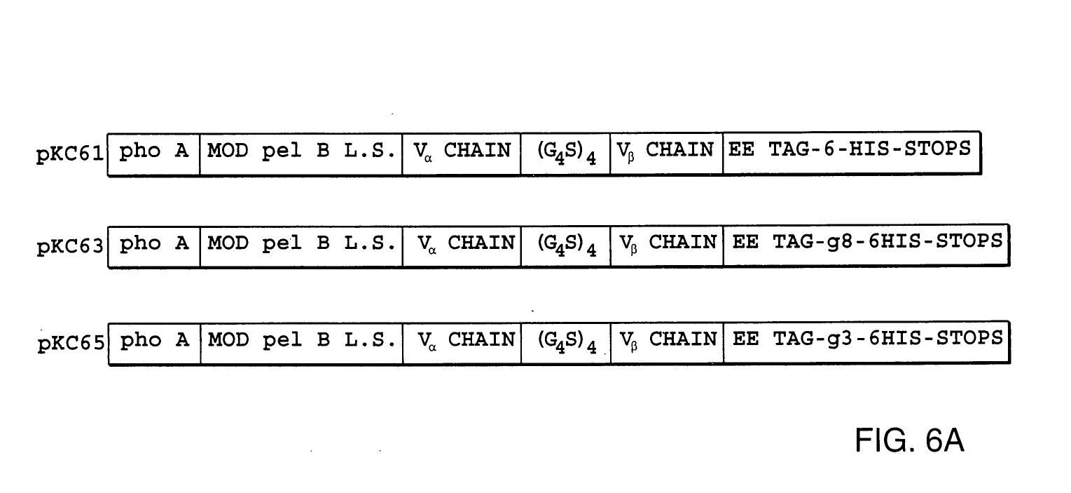

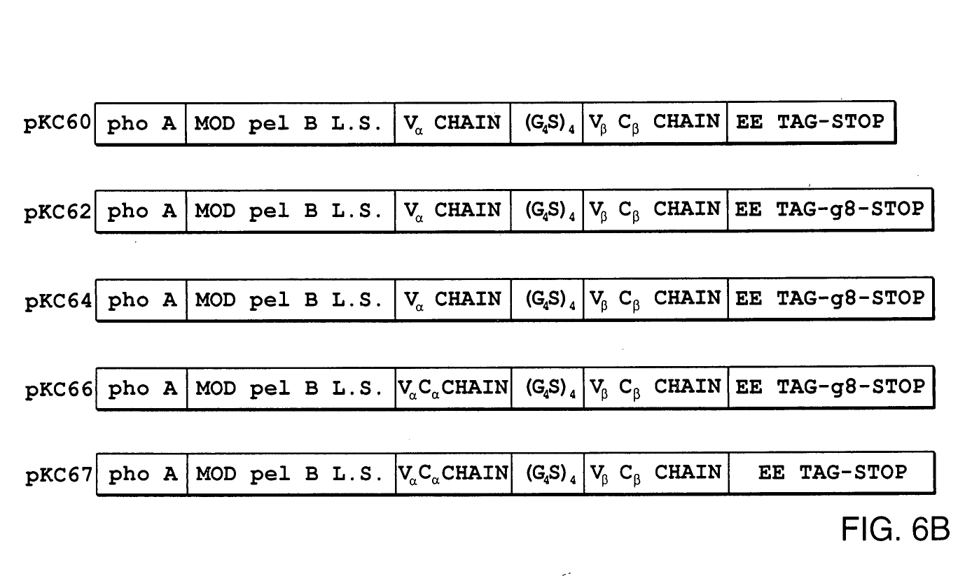

The scTCR was constructed to include a covalently linked V-α chain and V-β chain from the DO11.10 TCR. In some cases, the V-α chain further included the C-α chain fragment and the C-β chain fragment (See e.g, FIGS. 3, 6A and 6B). Generally, the Cα chain fragment was approximately 9 amino acids in length. The C-β chain was typically truncated at amino acid residue 126 just before the cysteine residue at amino acid residue 127 of the full-length C-β chain. It was found that inclusion of this cysteine residue was deleterious to scTCR expression. The 3′ end of the V-β chain (or C-β chain) fragment typically included a fused protein purification tag (e.g. EE, or 6XHis) DNA encoding a bacteriophage gene III or bacteriophage gene VIII coat protein.

### EXAMPLE 2

### Vectors for Expressing scTCR Fusion Proteins

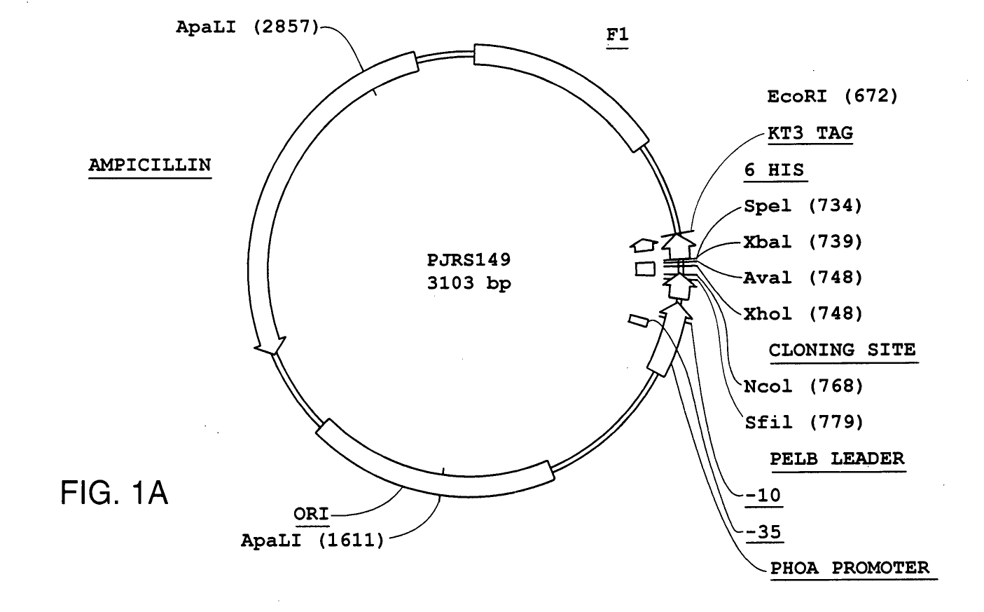

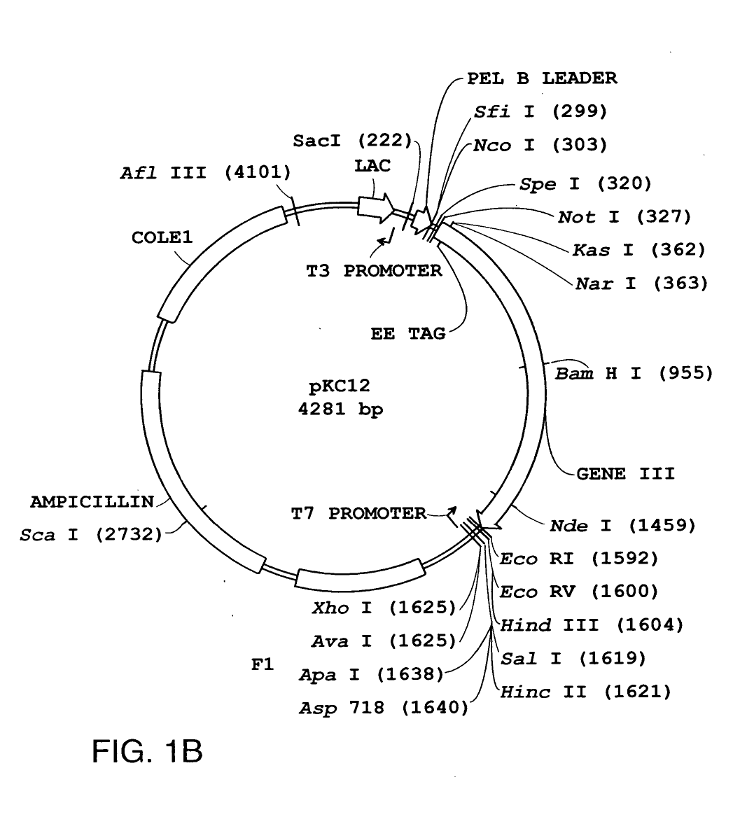

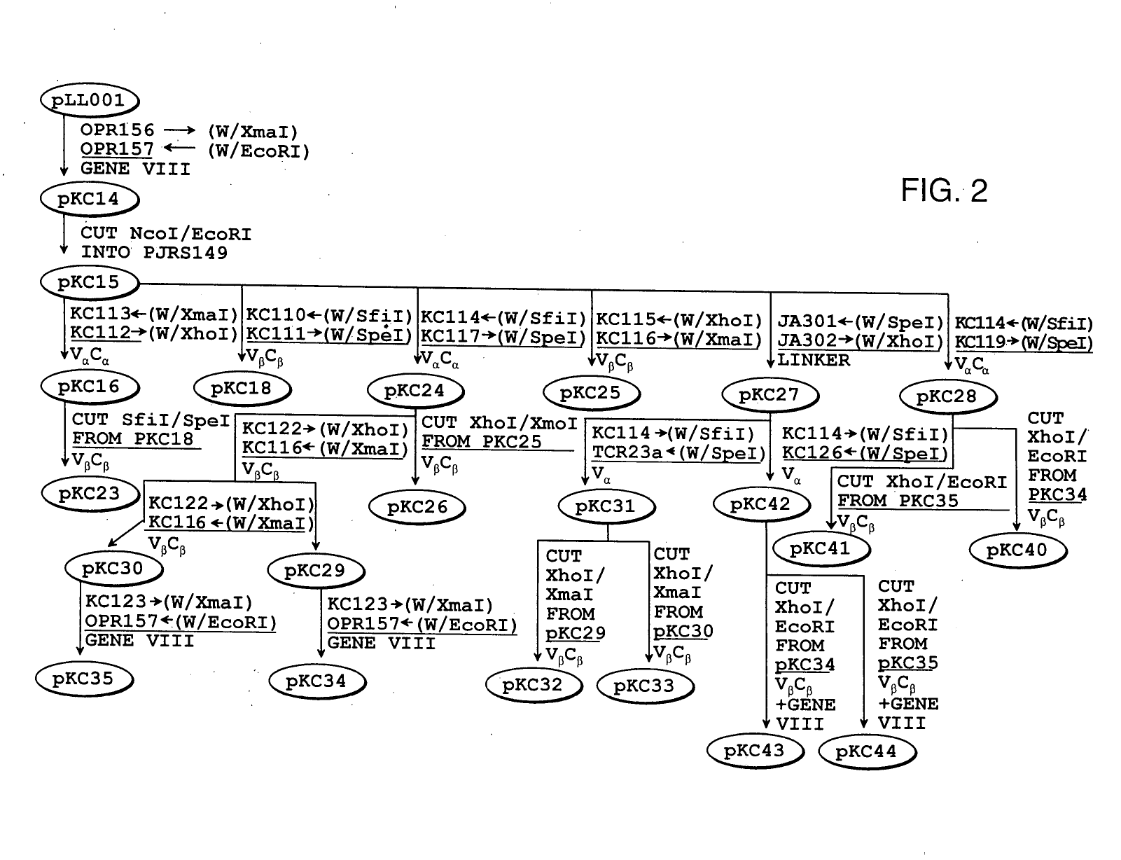

The scTCR DNA provided in Example 1 was inserted into vectors bearing a strong (phoA) or weak (lacZ) bacterial promoter. The vectors used to express the scTCR were the pJRS149 and pKC12 DNA vectors illustrated in FIGS. 1A and 1B, respectively. These vectors allow fusion of the scTCR to a bacteriophage coat protein. The construction of DNA vectors encoding fusion proteins is schematically outlined in FIG. 2 and as follows.

A. DNA Vector pJRS149
The pJRS149 DNA vector is a phagemid with a pBluScript™ (Invitrogen) backbone. The vector was used to produce soluble scTCR fusion proteins for use in bacteriophage display experiments described in Examples 14 to 18 which follows.

B. DNA Vector pKC12
DNA encoding gene III was cloned into the pKC12 vector illustrated in FIG. 1B.

C. DNA Vectors pKC14, and Vector pKC15
The gene VIII DNA sequence was PCR amplified from fd tet bacteriophage (ATCC No.37000) as template and primers OPR156 (“front” SEQ ID. NO.58) and OPR157 (“back” SEQ ID. NO. 59). The gene VIII PCR product was then cloned as a XmaI-EcoRI fragment into vector pLL001 to generate vector pKC14. The pLL001 plasmid was derived from PUVC-19 DNA and contained an XmaI and EcoRI site in the polylinker region for subcloning gene VIII segments. Additionally, the pLL001 vector was used as a shuttle vector to clone gene VIII DNA. Vector pKC15 was derived from pKC14 by cloning a 96 bp NcoI-EcoRI fragment into the pJRS149 vector shown in FIG. 1. The NcoI-EcoRI fragment contains a synthetic polylinker with multiple cloning sites (e.g., SfiI, NcoI, SpeI, XhoI and XmaI), a pelB leader, a phoA promoter and the gene VIII gene. Vector pKC15 has a second pelB leader which originated from the pJRS149 backbone and allows gene cloning to be under the control of a di-cistronic operon.

D. DNA Vectors pKC16 and pKC18
Alpha chain cDNA was used as template to amplify the TCR V-α, C-α gene using primers KC113 (SEQ ID NO: 6) (“front”) and KC112 (SEQ ID NO. 5) (“back”) for cloning a XhoI-XmaI fragment from pKC16. The V-β, C-β gene fragment was amplified using PCR and β-chain cDNA as template with primers KC110 (SEQ ID NO. 3) (“front”) and KC111 (SEQ ID NO:4) (“back” ) and cloned into pKC15 to form vector pKC18.

FIG. 3 illustrates the pKC44, pKC46 and pKC51 DNA vectors used in these experiments. The construction of the DNA vectors from pKC15 is outlined in FIG. 2 and described as follows.

E. DNA Vectors pKC27, pKC42 and pKC44
Vector pKC27 was constructed by annealing primers JA301 (SEQ ID NO. 95) and JA302 (SEQ ID NO. 96) to make a (G₄S)₄ polypeptide linker. The linker was subsequently cloned as a SpeI-XhoI fragment into the pKC15 vector. The V-α 13.1 and V-β, C-β domains were then each cloned into pKC27 DNA. Briefly, a V-α 13.1 gene fragment was produced by PCR amplification using primers KC114 (SEQ ID NO: 7) (“front”) and KC126 (“back”) (SEQ ID NO: 19) with vector pKC16 DNA as template. The V-α3.1 gene was cloned into pKC42 as a SfiI-SpeI fragment. V-β 8.2, and C-β chain DNA was gel purified after digesting pKC18 with XhoI and XmaI. The fragment was cloned into pKC42 to make the pKC44 vector. In general, the pKC44 vector included the scTCR as an scTCR/gene VIII fusion protein under the transcriptional control of the phoA promoter.

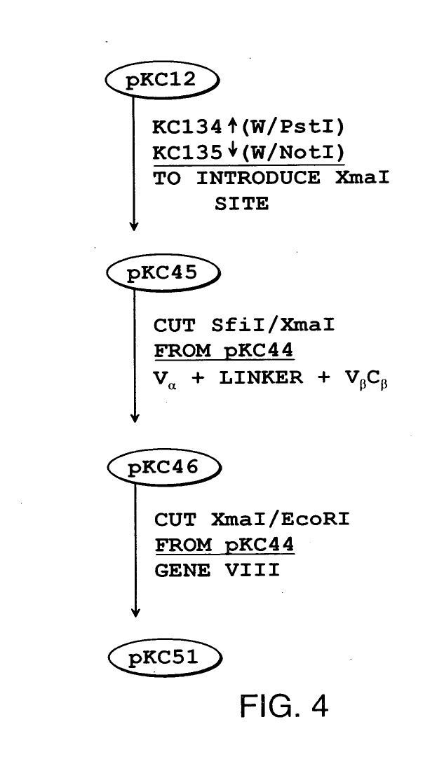

F. pKC12, pKC45, pKC46, and pKC51 DNA Vectors
The construction of vectors pKC45, pKC46, and pKC51 from pKC12 (FIG. 2) is outlined in FIG. 4 and described as follows.
The pKC12 vector was modified so that DNA encoding the scTCR from Example 1 was expressed under the transcriptional control of the lacZ promoter. Briefly, the pKC12 vector was modified by cloning annealed primers KC134 (SEQ ID NO: 27) (“front”) and KC135 (SEQ ID NO:28) (“back”) into pKC12 to produce vector pKC45. The modification added an XmaI site into the polylinker region of pKC12 which already included SfiI and EcoRI sites. Additionally, the pKC45 included a DNA sequence encoding gene III attached to the 5′ end of an EE-tag and an amber stop codon.
The amber stop codon reduced the level of scTCR fusion protein expression to approximately 15-20% in a lacI^{q} amber suppressor host such as XL1 -blue. To clone DNA encoding the scTCR/geneIII fusion protein into pKC45, pKC44 DNA was cleaved with SfiI and XmaI. The scTCR fragment was then gel purified and cloned into pKC45 to produce pKC46. The scTCR insert of pKC46 is illustrated in FIG. 3 fused to the EE-tag and gene III coat protein.
The scTCR/gene VIII fusion protein was placed under the transcriptional control of the lacZ promoter by digesting the pKC44 and pKC46 vectors with XmaI and EcoRI. The gene VIII DNA sequence was isolated from the digested pKC44 DNA and cloned into gel-purifed vector DNA pKC46 as an SfiI-EcoRI fragment to make vector pKC51. The scTCR insert in vector pKC51 was then fused to the gene VIII coat protein as schematically illustrated in FIG. 3c. Unlike vector pKC46, the pKC51 vector does not contain an EE-tag or an amber stop codon.

### EXAMPLE 3

### Cloning DNA Encoding Fusion Proteins into DNA Vector pEN2

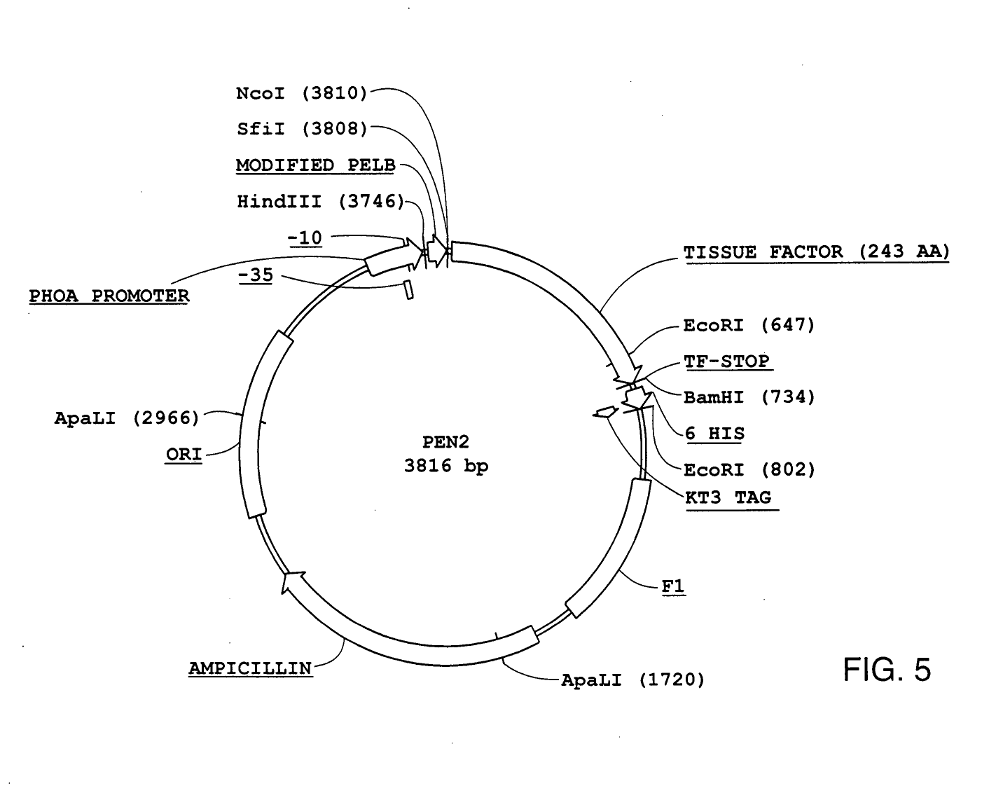

Expression of the soluble scTCR produced in Example 1 was increased by subcloning into the pEN2 vector illustrated in FIG. 5. The pEN2 vector includes a phoA promoter, a gene 10 ribosomal binding site, and a modified pel B leader. Soluble scTCR inserts in a pEN2 vector format are schematically illustrated in FIG. 6A and FIG. 6B and described as follows. In FIG. 6A and 6B, the DNA vectors were constructed from the pEN2 vector.

A. Construction of DNA Vector pKC60
The pKC60 vector was made by introducing a 1204 bp SfiI-EcoRI fragment into pEN2. The SfiI-EcoRI fragment consisted of the V-α and V-β, C-β domains. The fragment was made by amplifying pKC51 DNA as template and using primers KC114 (“front” SEQ ID NO: 7 ) and JWTCR208 (“back” SEQ ID NO:75). The JWTCR209 primer included a EE-tag and a XmaI site 3′ of the C-β domain. The addition of an XmaI site facilitated cloning of the gene III and gene VIII genes. The pKC60 vector was made by cloning the scTCR XmaI-EcoRI fragment described in above into the pEN2 vector.

B. Construction of DNA Vector pKC61
To create a truncated version of the scTCR, the V-α, V-β sequence of vector pKC46 was PCR-amplified with primers KC115 (“front” SEQ ID NO: 8 ) and JWTCR209 (“back” SEQ ID NO:74). The resulting PCR amplification products were subcloned into vector pKC60 to yield vector pKC61. The pKC61 vector was further modified by adding a 6XHis tag to the 3′ end of the EE-tag sequence.

C. Construction of pKC62 and pKC64 DNA Vectors
DNA encoding bacteriophage geneIII was amplified with primers TCR215 (SEQ ID NO 68) and 218 (SEQ ID NO 64) using pKC46 vector DNA as the template and cloned into pKC60 to make vector pKC64. The gene VIII gene was amplified with primers TCR212 (SEQ ID NO:71) and 213 (SEQ ID NO: 70) using vector pKC51 DNA as template and then cloned into pKC60 to make vector pKC62.

D. Construction of DNA Vectors pKC63 and pKC65
GeneIII (pKC65) and gene VIII (pKC63) fusion proteins are cloned into vector backbone pKC61 after PCR amplification of the respected gene fragments. Both pKC65 and pKC63 will then contain the 6XHis tail encoded in the primer sequence.

E. Construction of pKC66 and pKC67 DNA Vectors
The pKC66 and pKC67 vectors were made by amplifying an SfiI-SpeI fragment containing the V-α and the first 8 amino acids of the C-α chain fragment using pKC51 DNA as template and primers KC114 (front SEQ ID NO: 7) and JWTCR 217-β (back SEQ ID NO: 66). To make the pKC66 and pKC67 vectors, the KC114 and JWTCR 217-β primers were used to PCR-amplify TCR DNA, afterwhich the amplified DNA was subcloned into pKC62 or pKC60 which was previously digested with SfiI and SpeI These vectors each include 8 amino acid residues from the N-terminus of the C-α domain. The vectors were used in the construction of the TCR library described below. Vector pKC66 includes the gene VIII gene.

The DNA vectors pKC46 (pSUN18) and pKC62 (pSUN19) have been deposited pursuant to the Budapest Treaty with the American Type Culture Collection (ATCC) at 12301 Parklawn Drive, Rockville, Md. The DNA vectors were deposited with the ATCC on Feb. 26, 1997 and were assigned Accession Nos. 97895 (pSUN18) and 97896 (pSUN19). The DNA vector pKC62 (pSUN19) includes a phoA promoter, modified pelB sequence, gene 10 ribosome binding site and bacteriophage gene VIII promoter. The DNA vector pKC46 (pSUN18) includes the lac Z promoter, an EE tag and bacteriophage gene III protein. The DNA vectors can be propagated in E. coli or other suitable host cells in accordance with standard methods.

The DNA vectors pKC46 (pSUN18) and pKC62 (pSUN19) are designed to accommodate a variety of Vα, Vβ-Cβ and polypeptide linker sequences. The Vα chain of both DNA vectors can be removed by restriction digestion with SFiI and SpeI. The Vβ-Cβ chain can be removed by restriction digestion with XhoI-XmaI. Additionally, the DNA vectors allow exchange of the peptide linker sequence by restriction digestion with SpeI and XhoI.

### EXAMPLE 4

### Regulation of scTCR Fusion Protein Expression

1. phoA Promoter

XL1-B cells were transformed with vector pKC44 which includes the phoA promoter. The transformed cells were grown overnight in media containing phosphate to prevent scTCR fusion protein induction. When phosphate is removed from the media expression of the scTCR/geneVIII protein occurs. After overnight growth in shaker flasks the cells were washed several times in phosphate deficient media. Expression of the scTCR fusion protein was initiated by resuspending washed cells in phosphate deficient media. After a four hour induction, the cells were harvested. Typically, a four hour induction time gave suitable levels of soluble scTCR/gene VIII fusion protein as determined by Western blot analysis. Greater yields of the scTCR/gene VIII fusion protein (e.g., between approximately 10 to 400 mg) under phoA promoter control can be obtained by propagating cells in 3 and 10 liter fermentors or other large-scale preparative container such as a bioreactor.

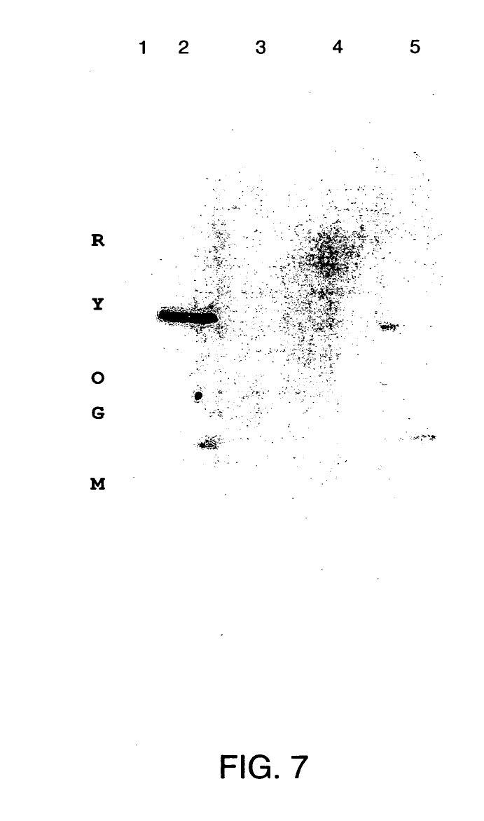

Generally, vectors that included a phoA promoter were preferred over vectors using other promoters such as Lac. Expression with the pEN2 vector was particularly preferred for a number of reasons. For example, in addition to including a phoA promoter the pEN2 vector includes a gene 10 ribosomal binding site that augments translation. The pEN2 vector further includes the pelB leader peptide which was modified to include codons preferred by E. coli. The modified pel B leader sequence and peptide are illustrated in SEQ ID NO: 129 and 130. The Western Blot illustrated in FIG. 7 shows that these modifications significantly improved expression of scTCR fusion proteins by approximately 10-50 fold.

The Western blot shown in FIG. 7 was probed with an anti-EE tag antibody (1:5000 dilution) followed by a goat anti-mouse-HRP antibody (1:20,000 dilution). Visualization was in accordance with standard immunological methods. Lane 1 shows molecular weight markers (Amersham). Lane 2 shows 5 microliters of 10 OD/ml of pKC60 scTCR (induced). Lane 3 is the same as lane 2, except uninduced (i.e., derepressed). Lane 4 shows 5 microliters of 10 OD/ml pKC51 scTCR (no EE tag). Lane 5 shows five microliters of 50 OD/ml pKC46 scTCR induced with IPTG.

2. LacZ Promoter

The scTCR/gene III (vector pKC46) and scTCR/gene VIII (vector pKC51) fusion proteins was expressed in approximately 3 liter fermentors. Expression of the scTCR fusion protein under the transcriptional control of the phoA promoter produced more fusion protein than vectors including a Lac promoter. Accordingly, the scTCR fusion protein was generally expressed from vectors including a phoA promoter.

### EXAMPLE 5

### Expression of scTCR Fusion Protein Strains

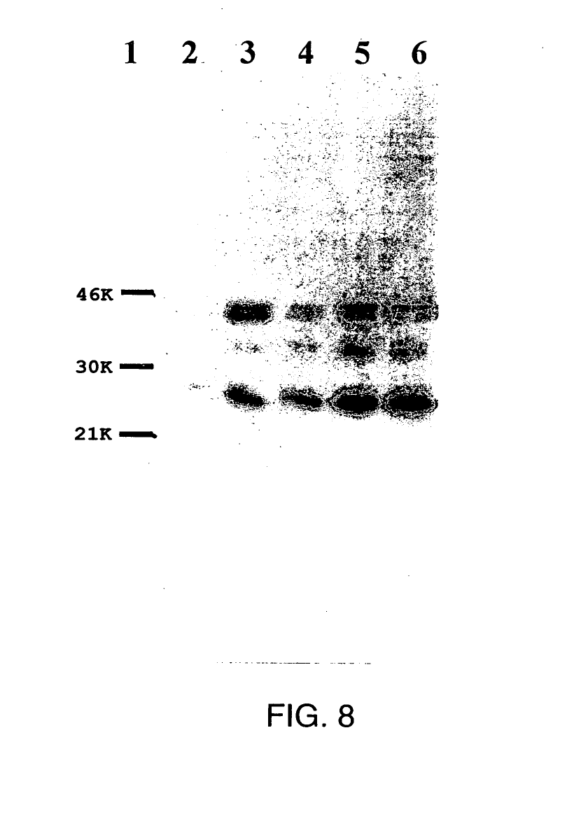

Several E. coli strains were analyzed for capacity to express scTCR fusion proteins (XL1-B, MM294, TB1, UT5600 and K91Kan). The host cell strains were transformed with pKC60 to express the scTCR fusion protein produced in Example 4. All transformed strains were adapted to grow in phosphate media at 30° C. Induction of the scTCR fusion proteins was preformed by growth in media deficient in phosphate. The level of scTCR expression was determined by Western blot analysis as shown in FIG. 8. In FIG. 8, lane 1 shows molecular weight markers (Amersham). Lane 2 shows four microliters of 10 OD/ml MM294 lysate (pKC60). Lane 3 is the same as lane 2, except that the host strain is K91kan. Lanes 4-6 are also the same as lane 2, except that the host strains are XL1-B, TB-1, and UT5600, respectively. All immunoprecipitations were prepared from phoA induced cells in the soluble cell fraction.

Briefly, the Western blot was conducted by adding 5 ul of a 5 OD/ml cell suspension of transformed cells on a 12% SDS-PAGE gel and transferred to a blot according to standard Western blot techniques. The scTCR was detected by probing the blot with anti-EE tag antibody licensed from Gernot Walter's laboratory at the University of San Diego, San Diego Calif. Alternatively, other anti-EE tag antibody and EE-tags can be used (e.g., those obtained from Pharmacia). Antibody binding was followed by the addition of a goat anti-mouse-HRP labeled conjugate (Jackson Laboratories). Expression of the scTCR fusion protein was greatest in strain K91 Kan (FIG. 8).

### EXAMPLE 6

### Purification of scTCR Fusion Proteins

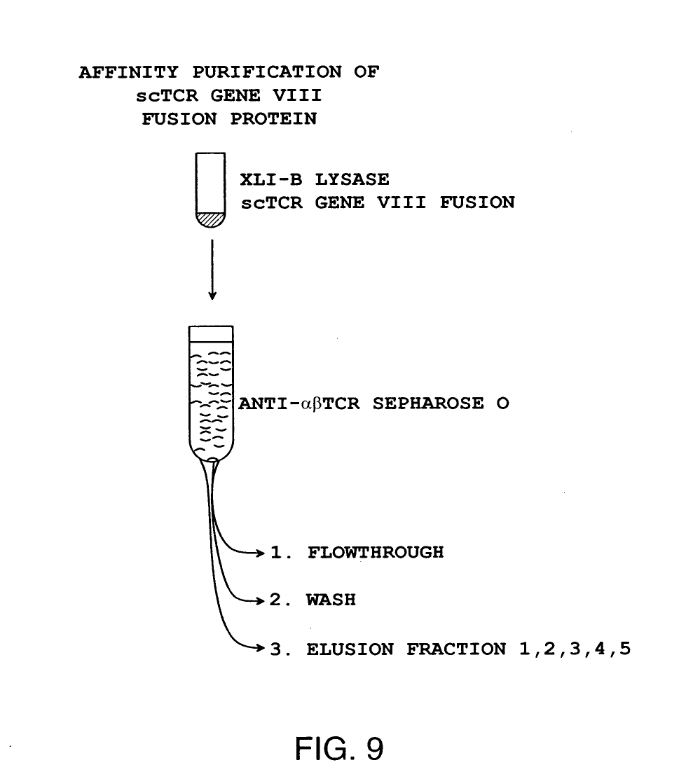

The fusion protein encoded by vector pKC51 was purified from transformed cells by immunoaffinity chromatography in accordance with conventional methods. The immunoaffinity purification of the scTCR/gene VIII fusion protein is schematically illustrated in FIG. 9.

Briefly, the purification was performed by making a chromatographic column by coupling 5 mg of hamster anti-mouse αβ TCR antibody H57-597 (ATTC Accession HB-218) per ml of protein-A coated sepharose beads. E. coli lysates were prepared by solubilizing 50 g of fermentor-derived cell paste in 100 ml of solubilization buffer (0.05 M Tris, pH 8.0, 150 mM NaCl and 5 mM EDTA. Resuspended cells were lysed by two passages through a French Press. Insoluble material was removed by centrifugation at 10,000 g for 20 minutes. The supernatant was retained and applied to the antibody column at a flow rate of 0.2 m/min. Subsequently, the column was washed with 20 column volumes of PBS and bound scTCR fusion proteins were eluted in 0.1 M glycine pH 3.0 and one ml fractions were collected in tubes containing 0.05 ml of 2 M Tris, pH 8.0. Fractions containing scTCR were pooled and dialyzed overnight against 4 liters of PBS. The next day purified protein was concentrated 5 to 10 fold using a centricon filtration device (mw 100 cutoff).

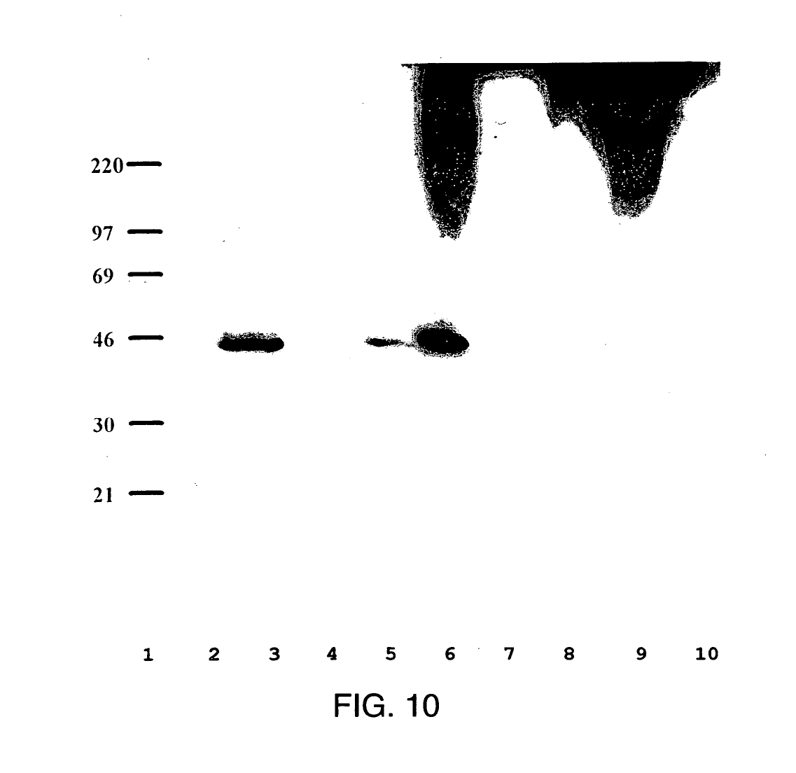

The scTCR fusion protein preparations were evaluated for purity by electrophoresis on an SDS-PAGE gel followed by commassie brilliant blue staining. Protein integrity was determined by western blotting using either antibody H57-597 or anti-Glu-Glu (EE) tag antibody as a probe. The EE antibody recognizes a nine amino acid linear epitope, EEEEYMPME (SEQ ID NO 98) (FIG. 10). Two other antibodies which bind to V-β8.2 conformational epitopes (MR5-2 and F23.1 antibodies (PharMingen)) were also used to purify scTCR fusion proteins. Additionally, a 6XHis tail was added to some constructs (see FIG. 6A) to facilitate scTCR purification by Ni²⁺NTA affinity chromatography.

FIG. 10 illustrates a Western blot showing expression of various scTCR fusion proteins from pKC51 (lane 2), pKC46 (lane 3), pKC46 purified on an H57 antibody column (lane 5), pKC46 re-folded and purified on an H57 antibody column (lane 6). In lane 7, approximately 5× more fusion protein was loaded than lane 1 or 6. Molecular weight markers are shown (lane 1). Flow through from a soluble (lane 9) and insoluble (lane 10) column fraction are also shown. Lanes 4 and 8 are blanks. The blot was probed with a 1:5000 dilution of anti-EE tag monoclonal antibody followed by visualization with a 1:20,000 dilution of goat anti-mouse-HRP antibody. Staining was conducted by standard methods.

### EXAMPLE 7

### Characterization of scTCR Fusion Proteins

A panel of anti-αβ TCR mAbs was used to characterize scTCR fusion proteins. The hamster mAb, H57-597, recognizes an epitope on the C-β domain. We conducted competition tests which showed a competing effect between MR5-2 and H57-597 suggesting that the epitopes bound by each antibody were in close proximity. Other antibodies were used to characterize the scTCR, including the nine amino acid EE sequence engineered into same TCR molecules and a polyclonal sheep anti-bacteriophage serum (Pharmacia) having broad specificity to gene III and gene VIII proteins.

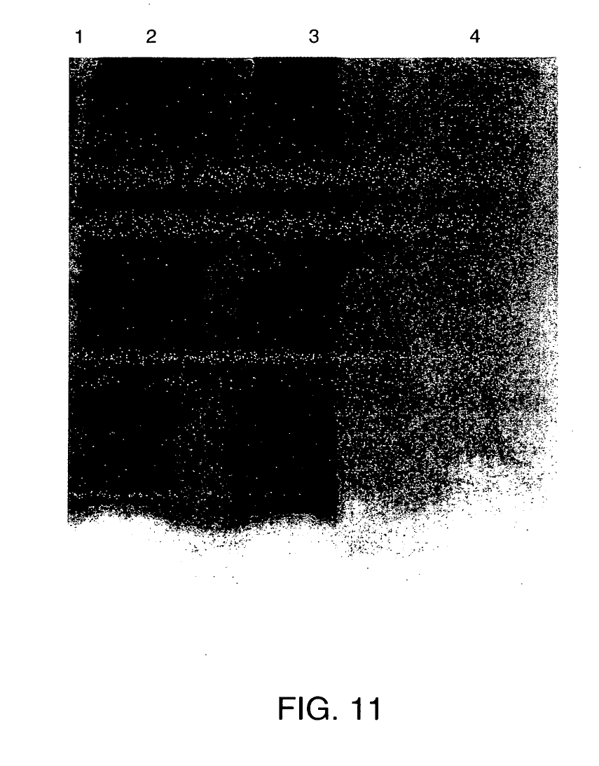

A. Molecular Weight
The scTCR/gene VIII fusion protein expressed from vector pKC51 was run on a 12% SDS-PAGE gel and stained with coomassie blue (see FIG. 11). In FIG. 11, lane 2 shows the scTCR fusion protein produced from a 10 OD/ml culture of XL1-B cells. Lane 3 of FIG. 11 shows flow through from an H57 antibody column used to purify the scTCR. Lane 4 of the figure shows the purified scTCR from the column. Lane 1 of FIG. 11 shows molecular weight markers. Inspection of the gel revealed that the scTCR fusion protein migrated in the gel at approximately 46 Kd. This result indicated that the scTCR was intact.

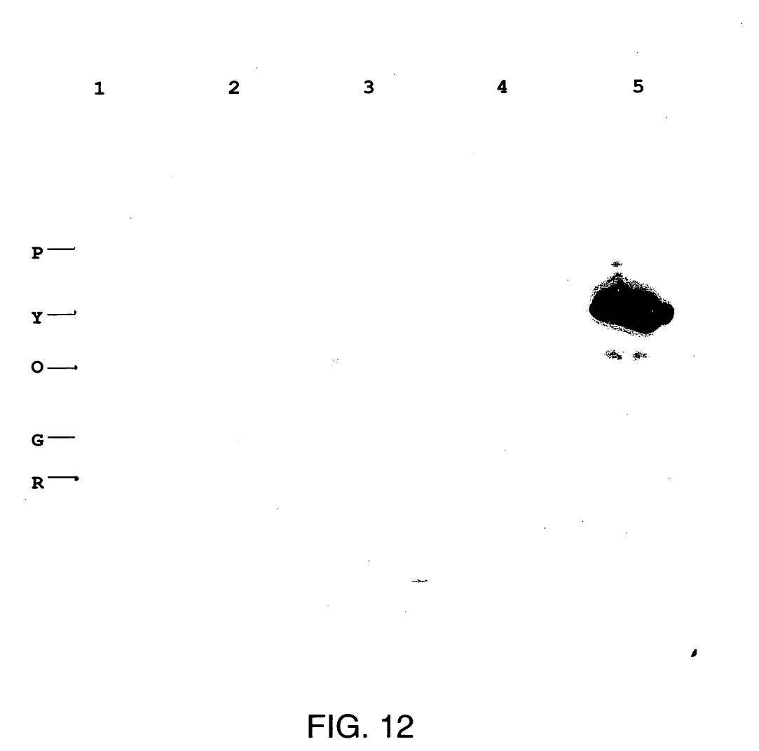

The Western blot shown in FIG. 12 was probed with antibodies which bind to a EE-tag, bacteriophage protein or an epitope within the C-β domain showed a 46-50 Kd protein (FIG. 12). scTCR fusion proteins produced from other DNA vectors were analyzed in a similar fashion.

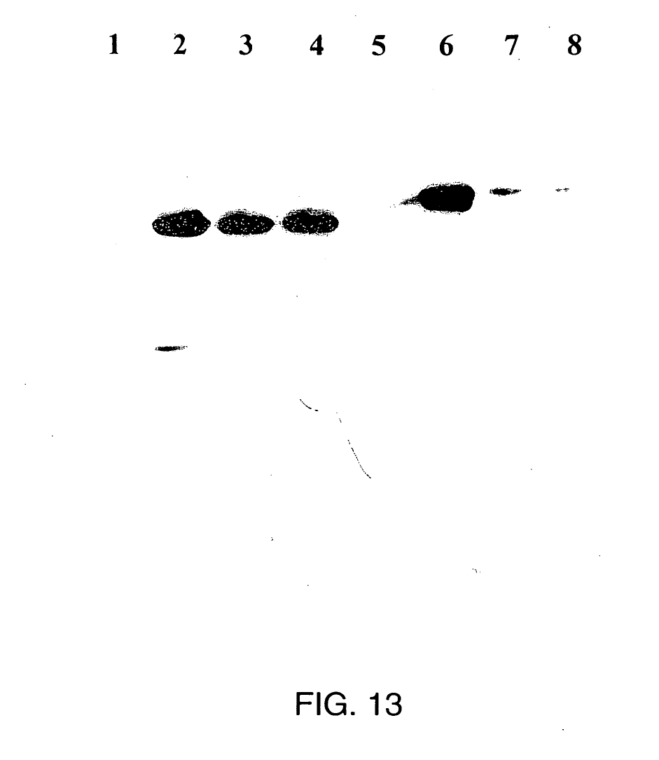

B. Conformational Folding
The fusion protein encoded by the pKC60 DNA vector was also tested for proper folding by performing binding assays with the anti-V-β8.2 antibodies (MR5-2 and F23.1) and with anti-idotype mab KJ1 (a kind gift from Drs. Kappler and Marrack). The scTCR fusion protein was incubated with the anti-V-β 8.2 antibody overnight at 4° C., followed the next day by binding with goat anti-mouse coated magnetic beads (Dynal). The material was allowed to incubate for an additional hour at room temperature (RT). Immune complexes were precipitated according to standard procedures. Beads were then extensively washed with 0.5 ml of PBS+0.5 M NaCl to remove non-specifically bound proteins. After 4 washes, the magnetic beads were resuspended in 50 ul of cracking buffer containing SDS with and without β-2 mercaptoethanol and boiled for 3 minutes. The magnetic beads were removed and the solution was loaded onto a 12% SDS-PAGE and run for 1 hour at 120 volts. The samples were electrophoresd and a Western blot was performed on the samples by probing the blot with anti-TCR antibody-HRP labeled (H57 obtained from Pharmingen) or anti-EE tag antibody (FIG. 13).

FIG. 13 is a Western blot probed with a 1:5000 dilution of anti-EE tag antibody followed by visualization with a 1:20,000 dilution of goat anti-mouse-HRP. Both cultures of pKC60 and pKC62 were induced by phosphate depletion. Lane 1 shows 10 microliters of a 10 OD/M1/sample of pKC60 soluble fraction. Lane 2 shows immunoprecipitation of the scTCR fusion protein produced from pKC60 by the MR5-2 monoclonal antibody. Lane 3 shows the same immunoprecipitation conditions as lane 2, except with MAb F23.1. Lane 4 shows immunoprecipitation of the scTCR fusion protein produced from pKC60 with MAb KJ1. Lanes 5-8 are the same as lanes 1-4 except the scTCR fusion protein from pKC62 was analyzed. All lanes were from induced cultures. The data indicates that the scTCR fusion protein had a conformationally correct V-β domain.

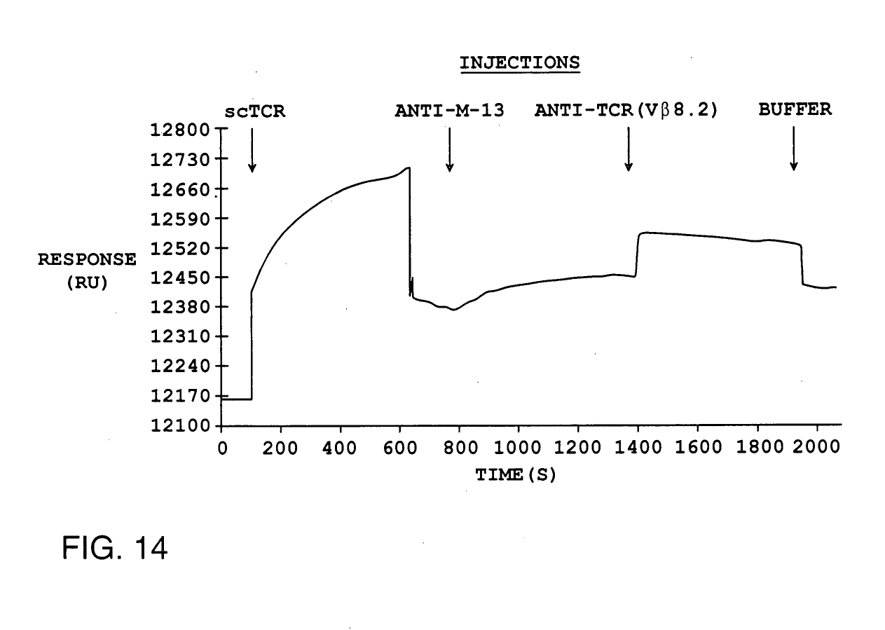

3. Surface Plasmon Resonance (Bia(Core)
The scTCR fusion protein encoded by pKC51 was further characterized using surface plasmon resonance (FIG. 14). Fusion proteins were detected in a conventional sandwich assay by first capturing the scTCR molecule on a biosensor chip coated with either MR5-2 or H57 antibody in general accordance with the manufacturer's instructions (Pharmacia). In general, if one of the antibodies was used to capture a scTCR fusion protein, the other antibody was used to form the sandwich complex.

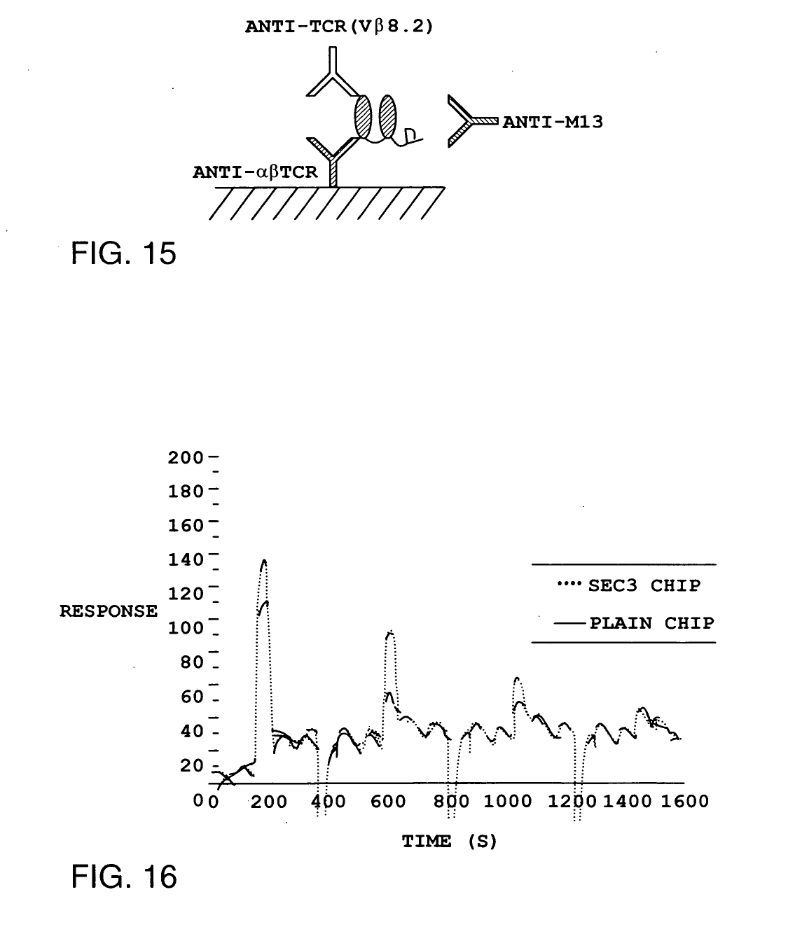

The data indicated that the two antibodies recognized different regions of β chain and competed for binding to the scTCR (FIG. 14). The interaction is schematically represented in FIG. 15. The scTCR could be further bound by the anti-M13 antibody indicating the presence of bacteriophage protein on the scTCR fusion protein.
Superantigens (ie. SAg) are capable of binding some V-β chains independent of presentation in an MHC format. The interaction between TCR and SAg occurs within the hypervariable 4 (HV4) domain of the V-β chain. Since SAg interaction with the HV4 domain is conformationally dependent, surface plasmon resonance was used to determine if the scTCR fusion protein could bind to SAg coated on chips (FIG. 16). SAgs are known to bind TCR V-β8.2. The highest affinity reported using surface plasmon resonance is believed to be 5×10⁻⁵ moles, thereby making the interaction comparable to TCR-MHC/peptide interactions.
The Streptococcus SAg known as SEC3 (Toxin Technology, Tampa Fla.) was coupled to a chip using standard amine coupling chemistry. The purified scTCR fusion protein was passed over the chip at a flow rate of 2 ul/min at concentrations ranging from approximately 2 uM to 16 uM. As a control, non-specific binding of the fusion protein to a blank chip was done in parallel with experiments measuring specific binding to the SEC3 coated chip. Binding data was compiled by comparing the difference in scTCR binding between the two chips. The data showed that there was a specific interaction between scTCR fusion protein and the SEC3 SAg on the chip. These data further support the antibody binding data and indicate that the scTCR included a conformationally correct V-β8.2 domain (FIG. 16).

### EXAMPLE 8

### Activity of Fusion Proteins in an OVA Specific T Cell Hybridoma Binding Assay

Cell-based competition inhibition assays are used to determine the function and biological activity of scTCR fusion proteins. In general, the fusion protein purified in Example 4 above (derived from the T cell hybridoma DO11.10) is used to block DO11.10 cells from engaging antigen in the context of an MHC molecule. The interaction is detected by measuring IL-2 production.

As described above, examples of assays for testing the functionality of scTCR fusion proteins have been disclosed in said published PCT Application No. US95/09816, as well as said pending U.S. patent application Ser. Nos. 08/382,454 and 08/596,387. More particularly, the said pending U.S. patent application Ser. No. 08/596,387 disclose T cell hybridoma cell assays, cells for use in such assays (e.g., D011.10), sc-MHC class I and II molecules, particularly sc-MHC IA_{d} class II molecules carrying a covalently linked or non-covalently bound presenting peptide (e.g., OVA or HSV gD12 peptide).

A) T Cell Hybridoma Cell Assay
A T cell hybridoma assay to test the functionality of scTCR fusion protein molecules has been disclosed in said published PCT Application No. US95/09816, as well as said pending U.S. patent application Ser. Nos. 08/382,454 and 08/596,387.
Briefly, the DO11.10 T cell hybridoma line expresses a cell surface T cell receptor specific for the 17 amino acid OVA peptide fragment (amino acids 323-339) derived from chicken ovalbumin. The OVA peptide is presented to DO11.10 cells by APCs expressing the murine class II MHC molecule I-Ad. When the peptide is presented by the appropriate APC, DO11.10 cells respond by producing IL-2, which can then be used as a measure of T cell activation. An exemplary T cell hybridoma cell assay is described briefly as follows.

The sc-IAd/OVA complex is diluted in DPBS (without Mg2+ and Ca2+ ions) and passively coated to wells of a 96 well plate. Preferably, the sc-IAd/OVA peptide includes a covalently linked presenting peptide. After an overnight incubation at room temperature, the wells can be washed two times in DPBS w/o Mg2+ and Ca2+ ions. About 1×10⁵ DO11.10 cells are incubated in the presence or absence of wells coated with 0.5 ug MHC/peptide molecules for 4 or 7 hours at 37° C.

To demonstrate specificity of scTCR fusion proteins, a second T cell hybridoma can be used (gD) which also is IAd restricted but recognizes an HSV peptide. The gD T cell hybridoma has been disclosed in said pending U.S. patent application Ser. No. 08/596,387. This assay is used to demonstrate that scTCR fusion proteins are capable of inhibiting IL-2 production by DO11.10 hybridoma cells and have little if any inhibitory effect on IL-2 production by gD12 T cell hybridomas. Cultures are carried out in complete culture medium (RPMI 1640 supplemented with 10% FBS, penicillin/streptomycin and L-glutamine) in 96 well flat bottom microtiter plates. After either a 4 or 7 hour incubation, culture supernatant is assayed for the presence of IL-2 using an IL-2 sandwich ELISA.

A suitable IL-2 detection protocol has been disclosed in said published PCT Application No. published PCT/US95/09816, as well as said pending U.S. patent application Ser. Nos. 08/382,454 and 08/596,387. The IL-2 sandwich ELISA protocol conducted herein was essentially as described by PharMingen. Briefly, wells are coated with 50 ul of a 2 ug/ml rat anti-mouse IL-2 antibody. The antibody binds to the plastic by passive diffusion after an overnight incubation at 4° C. The plate is washed two times in PBS/0.5% Tween-20 and then 100 ul of cell culture supernatant is added to each well and allowed to incubate for four hours at room temperature. The plate is then washed in PBS/Tween six times prior to adding the second antibody which is a biotinylated rat anti-mouse IL-2 antibody. This antibody incubates at RT for one hour and the plate is then washed six times in PBS/Tween. Finally, 100 ul of 25 ug/ml of strepavidn-peroxidase is added to each well and incubated for 30 minutes at RT. After 8 washes in PBS/Tween, 100 ul of ABTS substrate is added and the signal is read at OD 405 nm. The concentration of IL-2 produced is quantitated by plotting against a IL-2 standard curve.

Optimal doses of I-A^{d}/peptide molecules for activation of DO11.10 and gD12 cells are those which provide slightly submaximal responses. Accordingly, the experiments can be conducted with wells coated with 0.5 ug of I-Ad/peptide and a 4 or 7 hour incubation.

Soluble scTCR fusion proteins of the present invention can be tested for ability to block IL-2 production in D011.10 cells over a concentration ranging from 10⁻⁹ to 10⁻⁴ M. Tests can be performed with approximately a 10:1 to 1:1 molar ratio of the soluble scTCR and the soluble I-Ad/peptide molecules coated on the plate. Concentrations can be adjusted as needed. Optimally, a decrease in DO11.10 IL-2 production compared to the gD12 IL-2 production following pre-incubation with the soluble scTCR fusion proteins will indicate that the soluble scTCR fusion proteins can suppress immune responses in a TCR specific manner.

Because the TCR has a low binding affinity for MHC/peptide, it may not always be possible to observe a decrease in IL-2 production using the scTCR fusion protein in inhibition assays. However, avidity of the interaction can be increased by making polyvalent scTCR fusion proteins. By increasing avidity of the scTCR fusion proteins (and hindering dissociation thereof), it is believed fewer TCRs will have the opportunity to bind the MHC/peptide complex.

### EXAMPLE 9

### Preparation of Polyvalent Fusion Proteins

Several well-known methods are available for making polyvalent molecules. Polyvalent scTCR fusion proteins can be produced by biotinylating the scTCR in accordance with standard methods, followed by cross-linking the molecule after strepavidin addition. Biotin-streptavidin conjugation produces the polyvalent scTCR fusion protein, typically in a tetrameric format.

Polyvalent scTCR fusion proteins can also be made by covalently linking the fusion protein to latex beads (Polysciences, Inc. Warrington, PA) in accordance with known methods (see e.g., Newman, S. L. et al. J. Immunol. (1995) 154, 753). For example, scTCR fusion proteins can be directly coupled to the beads through either amine groups or disulfide groups. Denaturation of the scTCR can be minimized by coating the latex bead with either strepavidin or an antibody which specifically binds the scTCR. For example, the EE-tag antibody can be used to coat the latex bead in cases where the scTCR includes an EE-tag as described above.

### EXAMPLE 10

### Effects of scTCR Fusion Protein on Antigen Stimulated T Cell Proliferation in vitro

The soluble fusion proteins made in the examples above can be tested for capacity to suppress antigen stimulated T cell proliferation. Examples of such tests are those disclosed in said published PCT Application No. US95/09816, as well as said pending U.S. patent application Ser. Nos. 08/382,454 and 08/596,387.

In one method disclosed in said published PCT Application No. US95/09816, as well as said pending U.S. patent application Ser. Nos. 08/382,454 and 08/596,387, T cells can be isolated from any mammals such as mice. For example, OVA-primed T cells can be obtained from BALB/c mice (MHC Class II: I-Ad) by immunizing with 50 ug of OVA 323-339-KLH in complete Freund's adjuvant, subcutaneously at the base of the tail (see Harlow and Lane, supra). For example, two immunizations can be performed at 7 day intervals and, one week after the second injection, mice can be sacrificed and the inguinal and parasitic lymph nodes removed and dispersed into a single cell suspension. Subsequently, the single cell suspension can be depleted of antigen presenting cells by incubation on nylon wool and Sephadex G-10 columns. The purified T cell populations can be further incubated either with Click's medium alone, or with scTCR fusion proteins dissolved in Click's medium.

Activated B cells from BALB/c mice can be used as antigen presenting cells in a conventional B cell proliferation assay. For example, B cells can be prepared by culturing spleen cells with 50 ug/ml of LPS (ie. liposaccharides) for 48 to 72 hours at which time activated cells can be isolated by density gradient centrifugation on Ficoll/Hypaque (Pharmacia). Activated B cells can then be pulsed with the OVA 323-339 peptide for 3 hours, washed extensively, fixed with paraformaldehyde to inhibit proliferation of B cells, and added to purified T cells alone or T cells plus soluble scTCR fusion proteins. See generally Selected Methods in Cellular Immunol. (1980) B. B. Mishell and S. M. Hiigi W.H. Freeman and Co. San Francisco.

A standard B cell proliferation assay can be carried out in 96 well round bottom microtiter plates at 37° C., 5% CO2 for 3-5 days. Wells can be pulsed with WST-1 (Boehringer Mannheim) reagent for 4 hours prior to termination of cultures. The optical density of the cultures can then be recorded. The degree of peptide-reactive T cell proliferation will be indicative of the T_{H} cell responses (ie. clonal expansion) that took place in the mice following immunization.

### EXAMPLE 11

### Effects of scTCR Fusion Proteins on Antigen Stimulated T Cell Proliferation in vivo

The soluble fusion proteins can be further tested for inhibition of T cell clonal expansion in vivo in accordance with methods disclosed previously in said published PCT Application No. US95/09816, as well as said pending U.S. patent application Ser. Nos. 08/382,454 and 08/596,387.

For example, three test groups can be set up as follows: 15 BALB/c mice can be injected intraperitoneally (IP) with approximately 10 to 100 ug of OVA 323-339-KLH conjugate in Complete Freund's adjuvant to induce an immune response to the OVA 323-339 peptide. On the day before immunization and 2 days after immunization with OVA-KLH, 5 of the mice can be injected IP with approximately 10 to 100 ug of the anti-OVA/I-Ad scTCR fusion protein in PBS. The scTCR will bind to the I-Ad/OVA MHC Class II molecules to reduce or eliminate TCR molecules on antigen-specific T cells from engaging I-Ad/OVA molecules on APCs. The remaining 10 mice can be used as controls. For example, 5 of the mice can receive PBS and the other 5 can receive a scTCR with a different specificity. The mice can be sacrificed 10 days after immunization. The lymph nodes can be removed and dispersed into a single cell suspension. The suspension can be depleted of antigen presenting cells by incubation on nylon wool and Sephadex G-10 columns.

The resulting purified T cell populations can be incubated with APCs pulsed with the OVA 323-339 peptide. Activated B cells from BALB/c mice can be used as APCs in the proliferation assay. The B cells can be prepared by culturing mouse spleen cells with 50 ug/ml of LPS for 48 to 72 hours at which time activated cells can be isolated by density gradient centrifugation on Lymphoprep. Activated B cells can then be pulsed with the OVA 323-339 peptide for 3 hours, washed extensively, fixed with paraformaldehyde to inhibit proliferation of B cells, and added to purified T cells.

The T cell proliferation assay can be carried out in 96 well round bottom microtiter plates at 37° C., 5% CO2 for 3-5 days. Wells can be pulsed with WST-1 reagent for 4 hours prior to termination of cultures and then read at different absorbencies. The degree of peptide-reactive T cell proliferation in this assay will be indicative of the Tₕ cell response (ie. clonal expansion) that took place in the mice following immunization. It is expected that co-injection of the scTCR fusion proteins with either the OVA-KLH or the HSV-KLH immunization will limit the amount of clonal expansion and subsequent in vitro proliferation of the OVA-reactive T cell lines without affecting expansion of the HSV-reactive T cell lines.

### EXAMPLE 12

### Suppression of a Murine Autoimmune Disease

Experimental allergic encephalomyelitis (EAE) is a murine autoimmune disease that is generally recognized to be an animal model for multiple sclerosis. Encephalitogenic regions of two proteins, myelin basic protein (MBP amino acids 91-103) and proteolipoprotein (PLP amino acids 139-151) have been defined. See generally Martin, R. et al. Ann. Rev. Immunol. (1992) 10: 153.

In the SJL mouse strain, EAE can be induced to develop following immunization with the encephalitogenic peptide or adoptive transfer of MBP-reactive T cells. To determine whether treatment with soluble anti-MBP 91-103 or anti-PLP 139-151 T cell receptors will prevent EAE development after T cell activation, SJL mice can be injected with MBP 91-103 and PLP 139-151 reactive T cell blasts in vivo. Suitable assays to detect T cell expansion in such mice have been disclosed in said published PCT Application No. US95/09816, as well as said pending U.S. patent application Ser. Nos. 08/382,454 and 08/596,387.

As further disclosed in said published PCT Application No. US95/09816, as well as said pending U.S. patent application Ser. Nos. 08/382,454 and 08/596,387, EAE can be induced in SJL mice by immunization with approximately 400 ug of MBP 91-103 in complete Freund's adjuvant on the dorsum. Ten to 14 days later, regional draining lymph node cells can be harvested as described above and cultured in 24-well plates at a concentration of 6×10⁶ cells per well in 1.5 ml of RPMI 1640 medium/10% fetal bovine serum/1% penicillin/strepomycin/MBP at 50 ug/ml. After a 4 day in vitro stimulation, MBP 91-103 reactive T cell blasts can be harvested via a Ficoll/Hypaque density gradient (Pharmacia), washed twice in PBS, and 1.3×10⁷ cells can be injected into each mouse.

Mice which receive encephalitogenic MBP 91-103 reactive T cells can be further injected with approximately 100 ug of an scTCR fusion protein specific for MBP 91-103 (IAs context), 100 ug of an scTCR fusion protein specific for the PLP 139-151 (negative control), or saline (sham control) on day 0, 3, and 7 i.v. (total dose 300 ug). Clinical and histological evaluation can be performed to confirm that the scTCR molecule reactive to MBP 91-103+IAs inhibited the development of EAE in the mice.

As further disclosed in said published PCT Application No. published PCT/US95/09816, as well as said pending U.S. patent application Ser. Nos. 08/382,454 and 08/596,387, to induce EAE in SJL mice with PLP peptide 139-151, the mice can be immunized with PLP peptide 139-151 dissolved in PBS and mixed with complete Freund's adjuvant containing Mycobacterium tuberculosis H37Ra at 4 mg/ml in a 1:1 ratio. Mice can then be injected with 152 ug of peptide adjuvant mixture. On the same day and 48 hrs later, all animals can be given 400 ng of pertussis toxin. Adoptive transfer of EAE is then performed as described above.

In accordance with methods described above (see Example 1), TCR DNA can be obtained from normal and EAE diseased SJC mice. The TCR DNA can then be used to construct scTCRs which can be ligated into suitable DNA vectors comprising a bacteriophage coat protein or suitable fragment thereof. The PLP or MBP reactive scTCR peptide fusions can then be expressed and purified if desired in accordance with the above examples. The scTCR peptide fusion proteins can then be tested for the capacity to prevent development of EAE.

As described below in Examples 14 and 15 which follow, the PLP and MBP scTCR fusion proteins can be used to make a bacteriophage display library. The scTCR bacteriophage display library can be screened for receptors that can bind to either the MBP (91-103) or PLP (139-151) peptide in association with IA^{s}. As described more fully below in Examples 15, bound scTCR fusion proteins can be isolated by suitable panning techniques. The scTCR fusion proteins thus obtained can be used for production of soluble scTCR fusion proteins which can then be evaluated for blocking autoreactive T cells in SJL mice.

### EXAMPLE 13

### Affinity Maturation of scTCR Antigen Binding Domains

It is expected that high affinity scTCR fusion proteins can reduce or eliminate undesirable interactions between TCRs and MHC/peptide complexes such as those occurring in autoimmune disorders, allergies, and transplantation rejection. A high affinity scTCR fusion protein can be used, e.g., as a competitor for the corresponding native TCR, thereby reducing or eliminating binding between T cells bearing the TCR and APCs bearing MHC/peptide molecules. Alternatively, the scTCR molecule can reduce binding to a superantigen. It is believed that the low binding affinity and rapid off-rate associated with native TCRs will allow many scTCR fusion proteins to interact with a single MHC/peptide molecule.

High affinity scTCR fusion proteins can be used to reduce or eliminate binding of MHC/peptide molecules which activate autoreactive T cells. To accomplish this, genetic engineering (e.g., site directed or linker scanning mutagenesis) can be used to improve the affinity of scTCR fusion proteins, particularly by increasing the off-rate. Conventional binding assays have been developed to study protein off-rates. In cases where the sequence of an antigenic peptide which specifically binds the scTCR is known or can be readily determined, the peptide can be mutagenized by, e.g., alanine scanning mutagenesis, to identify residues which specifically bind the CDR3 region of the scTCR. Binding between the mutagenized antigenic peptide and the scTCR can be evaluated by binding assays described herein. Accordingly, amino acid residues identified in the peptide will make it possible to identify contact residues in the scTCR molecule. Preferably, a scTCR mutein produced by these methods will have the binding specificity of a TCR for antigen and the affinity of an antibody. One method of making the improved scTCR fusion proteins is by producing fusion protein muteins as described previously.

The improved scTCR fusion proteins can be made by isolating autoreactive scTCR fusion proteins using bacteriophage display libraries described in the examples which follow. The bacteriophage display libraries can then be used to pan against IA^{s} molecules covalently linked to peptides derived from PLP and MBP proteins. Once a scTCR is isolated, scTCR muteins can be made as described previously to further characterize peptide contact residues.

### EXAMPLE 14

### Production of Bacteriophage Display Libraries

The DNA vectors pKC46 and pKC51 described in Example 1, above, were used to display scTCR fusion proteins on the surface of bacteriophage after IPTG induction. The production of bacteriophage display libraries is generally well known and can be used to display polypeptides and proteins up to approximately 50 KD. See generally Smith, G. P. and Scott, J. K. in Methods in Enzymology (1993) 217: 228.

Briefly, a flask containing 2× Luria Broth (LB)+0.5% glucose and 100 ug/ml of ampicillin was inoculated with E. coli strain, XL1-B cells containing the plasmid vector pKC46 or pKC5 1. Following overnight growth, the cells were pelleted by centrifugation and resuspended in 2×LB without glucose. The cells were centrifuged again and washed a second time in 2×LB. After the final wash, cells were resuspended in 50 ml of 2×LB and IPTG was added to a final concentration of 1 mM. The cells were grown for 2 hours at 37° C. and then helper bacteriophage, VCSM13, was added at 10 pfu/5 ml of cell culture. After 15 minutes, the mixture of XL1-B cells and helper bacteriophage were diluted 1 to 50 into pre-warmed 2×LB containing tetracycline and ampicillin and 1 mM IPTG. After an hour of growth at 37° C. kanamycin was added to cultures to select for cells infected with helper bacteriophage. Cell cultures were grown overnight and bacteriophage were purified the next day after two rounds of PEG precipitation. To remove large debris from the sample, purified bacteriophage preparations were filtered through a 0.2 micron filter. Additionally, residual PEG was removed from the bacteriophage preparations by washing the sample in 1% FBS/PBS using a 100 centricon membrane (100 mw cutoff). The bacteriophage titer was determined by counting colonies growing on plates. This is done by making 10-fold serial dilutions of bacteriophage and mixing the diluted samples with infection competent XL1-B cells and plating contents on 2×LB agar containing ampicillin. The bacteriophage titres were in the range of approximately 10¹⁰ to 10¹⁴ cfu/cell.

Following the second PEG precipitation, bacteriophage were sterilized by filtration through a 0.2 micron filter to remove undesirable particles and bacteria. The filtered preparation was then washed exhaustively using a centricon 100 (100 mw cutoff) filter to concentrate the bacteriophage and to buffer exchange into 1% FBS/PBS.

### EXAMPLE 15

### Characterization of Bacteriophage Libraries

A) ELISA Assay
A TCR specific ELISA assay was used to test for TCR molecules displayed on the surface of bacteriophage. Briefly, a 96 well plate was coated with neutrAvidin (Pierce) at 200 ng/well in coating buffer, pH 9.0 and incubated at 4° C. overnight. The plate was blocked using 5% non-fat dry milk (NFDM) for one hour and then either anti-α,β TCR (H57) or anti-V-β8.2 (MR5-2) biotin labeled antibodies was diluted in 10 mM Tris, pH 8.0 containing 2.5% NFDM. The diluted antibodies were then individually added to the wells and incubated for one hour at RT. The plate was washed six times in TBS/Tween (0.5%) (TBST) to remove unbound antibody.

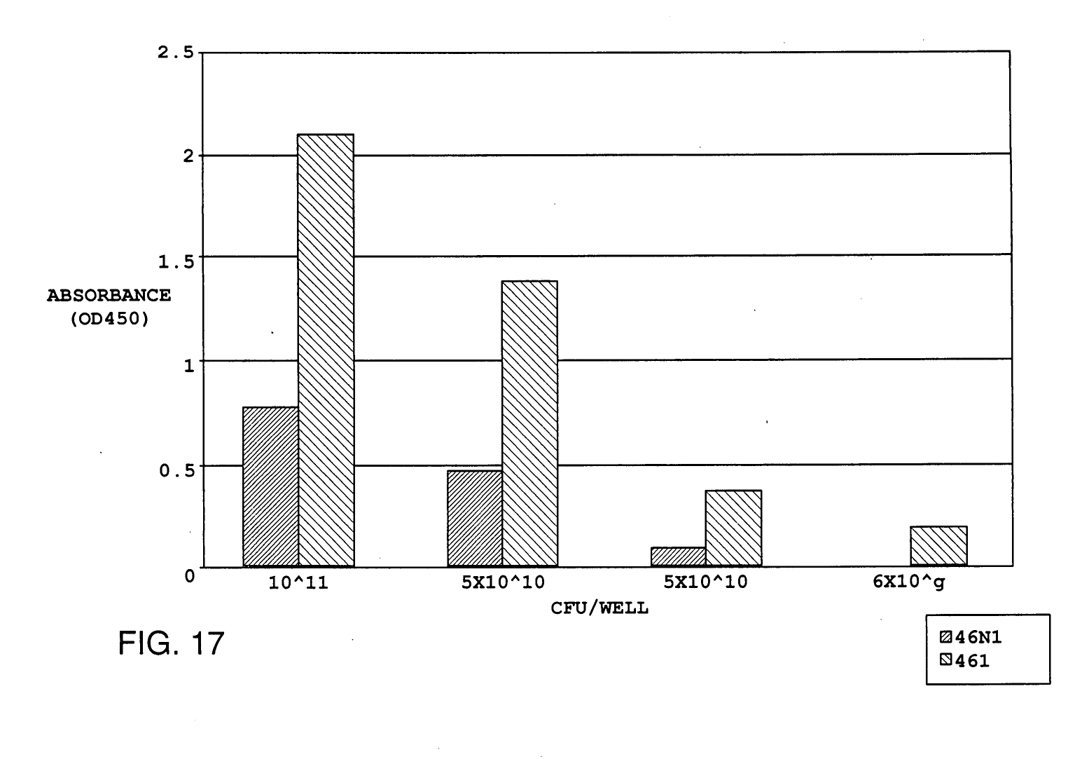

The bacteriophage-expressed fusion proteins were detected by incubating bacteriophage particles in wells coated with antibodies for one hr at RT. The plate is washed six times with TBST followed by the addition of anti-M13-HRP conjugate (Pharmacia) diluted in 2.5% NFDM. After a one hr incubation, the plate was washed eight times with TBST. 100 ul of TMB substrate was added to each well and after 10 minutes the reaction was quenched by the addition of 100 ul of 1M sulfuric acid. The plate was read at an absorbance OD of 450 nm (FIG. 17). We tested control bacteriophage (prepared from a CA III geneIII antibody library) for non-specific binding to antibodies H57 and MR5-2. Further controls were performed by assaying non-specific binding to wells coated w/BSA or with anti-V-β17.

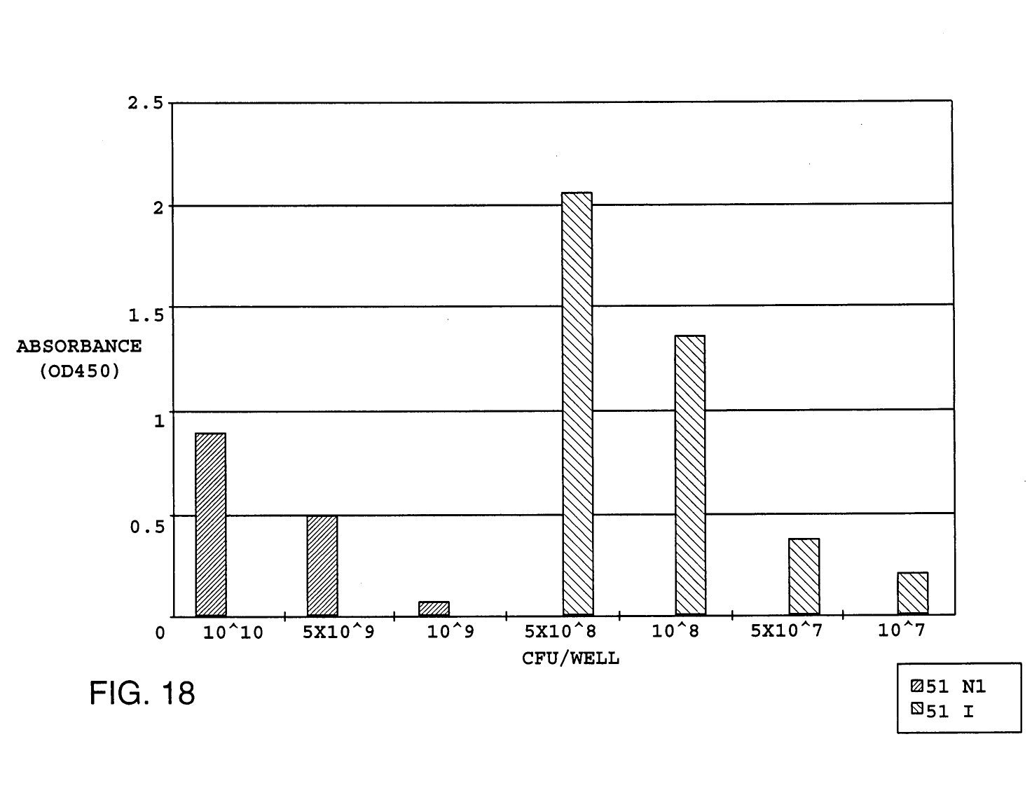

FIG. 17 and FIG. 18 show an ELISA assay of scTCR fusion proteins under non-induced (ie. derepressed) and induced conditions. FIG. 17 illustrates scTCR fusion protein made from pCK46 and FIG. 18 shows fusion protein made from pCK51. In each figure, dark boxes show non-induced cultures and light boxes show induced cultures. FIGS. 17 and 18 show that scTCR fusion proteins were expressed in the bacteriophage library and displayed on the surface of the bacteriophages. The OD 450 nm absorbencies were approximately 60 to 200 fold higher in bacteriophage preparations induced to express scTCR fusion proteins than non-induced bacteriophage preparations (FIG. 18). The ELISA assay detected bacteriophage displaying gene VIII scTCR fusion proteins at approximately 200 to 500 fold higher levels than bacteriophage displaying gene III scTCR fusion (see FIG. 17). It is believed that the results indicate that the TCR/gene VIII bacteriophage have multiple scTCR fusion proteins expressed on the bacteriophage surface. Multivalent bacteriophage display of scTCR fusion proteins would improve chances of panning against MHC/peptide complexes because the multivalency would increase avidity.

B) Activity of Fusion Proteins Displayed in the Bacteriophage Library

To demonstrate that the fusion proteins on bacteriophage were biologically active, bacteriophage displaying the DO11.10 scTCR/gene VIII fusion were analyzed for the capacity to block specific interactions between the TCR on the DO11.10 T cells and an immobilized scIAd/OVA molecule. Exemplary assays to detect such interactions using a DO11.10 T cell line and single-chain MHC molecules have been disclosed above.

A DO11.10 T cell hybridoma and IAd/OVA system was used in general accordance with the methods disclosed previously in Example 8 to measure the reduction of IL-2 levels in the presence of scTCR expressed on bacteriophage (sometimes referred to herein as “scTCR/bacteriophage molecules”). The experiments indicated that the bacteriophage-expressed scTCR fusion proteins interacted with immobilized Iad/OVA because IL-2 levels were reduced in wells that received scTCR/bacteriophage molecules. However, in wells incubated with equal titers of control bacteriophage (no displayed scTCR) a decrease in inhibition was not observed. Thus, the bacteriophage-expressed scTCR fusion proteins are biologically active.

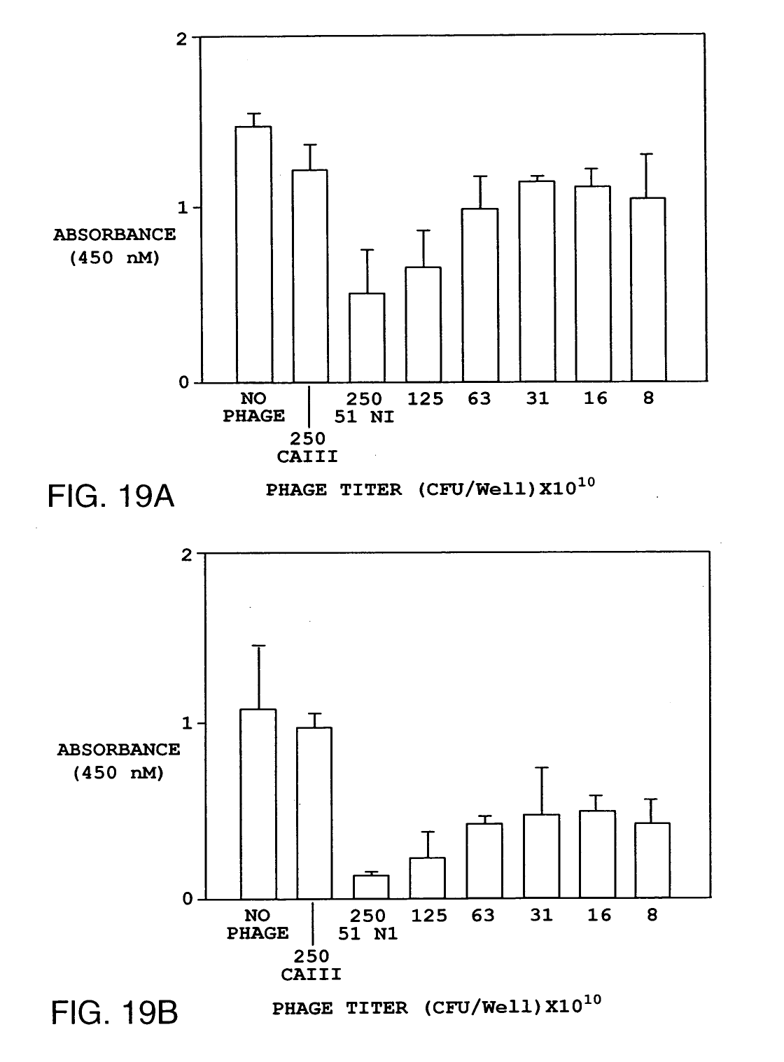

To test the fine specificity of the inhibition, an assay was developed that compared the level of inhibition between DO11.10 and gD12 T hybridomas. As disclosed previously in Example 8, gD12 T cell hybridomas are IA^{d} restricted but recognize a peptide derived from HSV-1. Because both cells recognize peptides restricted by IA^{d}, it is conceivable that the bacteriophage-expressed scTCR from DO11.10 could have some interaction with the IA^{d}/HSV-1 molecule, although it is believed that this interaction would be substantially weaker than the interaction with IA^{d}/OVA MHC/peptide molecule. As shown in FIG. 19A, a low level of IL-2 inhibition was observed in the gD12 hybridoma group, however, the level of IL-2 inhibition in the DO11.10 group was approximately 8 to 10 fold greater. In FIG. 19A, approximately 8 to 250×10¹⁰ phage were used in each experiment. CA^{III} refers to a control phage with no displayed scTCR fusion protein. FIG. 19B shows the results of a related experiment in which IL-2 production was measured from DO11.10 T hybridoma cells.

C) BioPanning Fusion Proteins

Bacteriophage display libraries that present scTCR fusion proteins can be used to pan for specific TCR molecules in accordance with known methods (see e.g., McCafferty, J. et al. Nature (1990) 348:352; Castagroli, L. et al. J. Mol. Biol. (1991) 222:301; Lebeddee, S. L. et al. PNAS (USA) (1992), 89: 3175; Smith, G. P. and Scott, J. K. supra; Blake, J. et al. J. Exp. Med. (1996) 184: 121. Prior panning techniques generally rely on the strength of interactions between a target antigen and antibody which is typically in the range of 10⁻⁶ to 10⁻⁸. However, most TCR-MHC/peptide interactions are weaker with KD values typically in the range of 5×10⁻⁵ M. In general, avidity and valency between scTCR fusion proteins and MHC/peptide complexes can be increased by increasing the number of fusion protein copies expressed on the phage. Other strategies which can be employed include changing wash stringency, increasing the amount of antigen, decreasing off-rate by lowering temperature, and increasing incubation time.

i)—-Antibody Panning

Antibodies specific to the DO11.10 scTCR fusion protein were used to pan for scTCR molecules. Briefly, microtire wells were coated with 200 ng of neutrAvidin followed by incubation with biotin labeled antibodies that bound to either the C-β domain, the V-β8.2 domain, or a non-specific antibody that recognized V-β17. Non-specific binding was also checked by use of wells coated with BSA. The experiments were conducted by diluting 2×10⁶ TCR/geneVIII bacteriophage particles into 1×10¹¹ particles from an irrelevant antibody bacteriophage bank, thus a final dilution of 1:50,000 was done. A 5000 fold enrichment was observed after a single round of enrichment against both the anti-TCR antibody (H57) or the anti-V-β8.2 antibody (MR5-2). The BSA negative control did not yield any positive clones suggesting the enrichment was specific. A first round enrichment between 20 to 10000 fold is not uncommon in antibody antigen panning experiments, indicating that observed enrichment falls within an exceptable range of reported enrichment studies.

ii)—Cell Panning

Cell panning is a routine method of isolating antibodies against cell surface proteins. The method can be readily adapted to screen for desired fusion proteins in the bacteriophage library made in Example 18. Cells for use in cell panning can be from any suitable source, e.g., cells which express MHC/peptide molecules or cells which include an empty MHC complex to which a suitable peptide can be bound as disclosed in said pending U.S. application Ser. No. 08/596,387. Additionally, cells can be employed that have been transfected with a single-chain class I or class II MHC gene in which a suitable peptide is bound or covalently linked to the MHC complex. Examples of cells with suitable single-chain class I or class II MHC/peptide complexes have been disclosed in said published PCT Application No. published PCT/US95/09816, as well as said pending U.S. patent application Ser. Nos. 08/382,454 and 08/596,387.

In general, panning with cells expressing a suitable MHC complex offers several advantages including promoting an available supply of such cells and avoiding time-consuming purification of the MHC/peptide complex.

In one cell panning method, approximately 10¹¹ total bacteriophage with approximately 2×10⁶ D011.10 bacteriophage can be mixed in an eppendorf tube (1.7 ml) with 10⁶ cells in a volume of 0.2 ml for a two hour incubation at 4° C. Cells can then be pelleted by centrifugation at 5,000×g for 10 minutes at 4° C. and washed 5 times in ice cold PBS/tween (0.5% w/v). Subsequently, bacteriophage can be eluted from the cells by adding 50 ul of citrate buffer, pH 5.0. The enriched bacteriophage population can be expanded by infecting E. coli and growing an overnight culture. Bacteriophage are then purified by standard procedures such as those described previously in Example 14. After the 5th round of enrichment, a random sample of colonies can be picked and the genes sequenced. The frequency of DO11.10 TCR genes recovered will determine whether additional rounds of panning are required. For instance, it is expected that a 1:50000 initial dilution of DO11.10 TCR/bacteriophage would show an increase in frequency after five rounds of panning. The increase is expected to be in the range of approximately 1000 to 2000 fold.

iii)—Panning with scMHC/Peptide Complexes

Soluble scMHC/peptide complexes can be made, e.g., in insect cells in accordance with methods previously described in said published PCT Application No. US95/09816, as well as said pending U.S. patent application Ser. Nos. 08/382,454 and 08/596,387.

Soluble scMHC/peptide molecules produced in insect cells can be used to pan for bacteriophage expressing soluble fusion proteins. For example, a concentration of scIA^{d}/OVA (e.g., 1 to 10 ug/well) can be used to coat 96 well plates (Nunc). Subsequently, 2×10⁶DO11.10 bacteriophage can be diluted (1:50000) into 10¹¹ total bacteriophage and incubated with immobilized IA_{d}/OVA. After a two hour incubation, the plate can be washed 5 times in TBST. Bound bacteriophage can be eluted by adding 100 ul of 0.1M HCl/glycine. The frequency of bound DO11.10 TCR/bacteriophage can be determined by performing standard colony lifts and screening for positives with an antibody against a protein tag (e.g., an anti-EE tag antibody as disclosed previously) or DNA specific probes, e.g., a labelled 300 bp α-chain DNA probe available from Amersham that recognizes the scTCR gene or gene product, respectively.

### EXAMPLE 16

### Fluorescence Assisted Cell Sorting Identification of Fusion Proteins

Fluorescence Assisted Cell Sorting (FACS) is a routine method for detecting and sorting cells (see e.g., Davey, H. M. and Kell, D. B in Microbiological Reviews (1996) 60: 641; and Darzynkiewca, Z. et al. (1994) in Flow Cytometry 2nd Ed., Vols. 41 and 42 Academic Press, New York. FACS can be used to detect interactions between cells and bacteriophage displaying scTCR fusion proteins. To perform FACS experiments, cells expressing sc-IA^{d}/OVA complexes were made as disclosed previously. Staining with the IA^{d} specific antibody ASMII-FITC (Pharmingen) verified that the cells expressed the IA^{d}. To confirm that the OVA peptide is present in the IA^{d} groove, a T cell activation assay can be performed as described in said published PCT Application No. published PCT/US95/09816, as well as said pending U.S. patent application Ser. Nos. 08/382,454 and 08/596,387.

A variety of chromagens can be used to label bacteriophages for FACS analysis. One approach is to couple bacteriophages with a suitable chromogen such as phycoerythrin.

Another approach is to couple the bacteriophages with FITC in accordance with known methods such as those described in the Flurotag FITC conjugation kit (Sigma, St. Louis, Mo.). Still another approach is to couple phycoerythrin to bacteriophages indirectly by first biotinylating bacteriophages and coupling strepavidn-phycoerythrin thereto. Still another method would be to use a two antibody approach in which each antibody had a different fluoroscein tag. More particularly, an anti-bacteriophage antibody can be labeled with FITC and a second antibody to the scTCR (e.g., H57) can be labeled with phycoerythrin.

One bacteriophage coupling method is as follows: 10¹² cfu of pKC51 phage are buffer exchanged into 0.1M sodium carbonate buffer (0.2 ml) at a pH of between 9.0 and 9.5. Using the Flurotag FITC conjugation kit, one FITC vial is mixed in 1 ml of 0.1M carbonate-bicarbonate buffer and vortexed until all FITC has been dissolved. Approximately 50 μl of FITC label is added to the bacteriophage preparation so that the final ratio of FITC to phage is 40 to 1 to obtain a suitable fluroescein to protein (F/P) molar ratio. The sample is incubated for 2 hrs in a tube covered with aluminum foil. Labelled bacteriophage is isolated by applying the sample to a G-25 Sephadex column. Typically, the labelled bacteriophage will be in the main fraction (e.g., fractions 6 to 8). The F/P molar ration is subsequently determined using a spectrophotometer to read absorbance at 280 nm and 495 nm. An equation that can be used to determine the F/P molar ratio is the following:
Molar F/P=2.77×A₄₉₅/A₂₈₀(0.35×A₄₉₅)

The absorbance reading of the conjugate sample should suitably fall between 0.3 and 1.0.

The FITC labelled phage can be incubated with the cells expressing the sc-IA_{d}/OVA complex in accordance with conventional FACS methods to detect bacteriophage binding to the cells. Bound bacteriophage can be eluted from the cells and propagated in accordance with standard methods.

### EXAMPLE 17

### Cloning and Expression of scTCR Fusion Proteins in Bacteriophage f88

The panning methods described above employ phagemid-transformed cells co-infected with a wild-type filamentous bacteriophage (ie. VCMS13) to display scTCR fusion proteins. The methods can be improved by using bacteriophage vectors which more efficiently package recombinant scTCR constructs (ie. result in a higher yield of recombinant molecules per total number of virions). For example, bacteriophage f88-4 is a type 88 vector (9273 base pairs) that includes two gene VIII genes, one which is wild-type, and the other which displays a recombinant gene (ie. a gene VIII gene fusion). The bacteriophage f88-4 can be obtained from Dr. G. Smith of the University of Washington. The f88-4 bacteriophage packaged gene VIII constructs more efficiently, resulting in up to 10% increases in yield. It is believed that the increase in yield followed an increase in the multiplicity of recombinant virions (ie. number of recombinant virions per total number of virions). It is believed that the increase resulted in approximately 300 scTCR fusion proteins per virion. Thus, the f88-4 bacteriophage increased yield and multiplicity, and improved avidity of target binding thereby enhancing the probability of isolating specific scTCR fusion proteins.

Recombinant bacteriophages including scTCR fusion proteins were constructed by cloning an scTCR gene into the PstI and HindIII sites of the f88-4 bacteriophage. Expression of the inserted scTCR gene is thus under the control of the tac promoter and induction occurs after the addition of 1 mM IPTG.

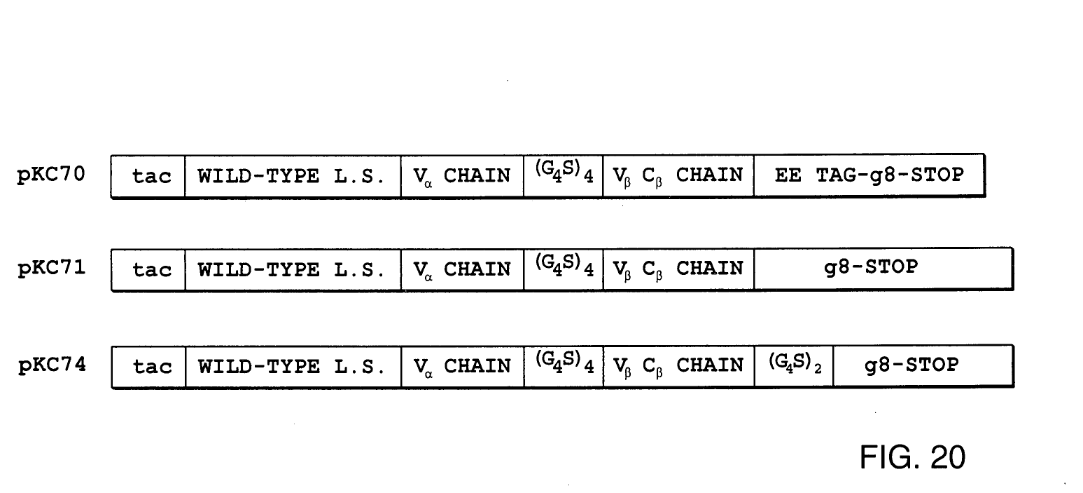

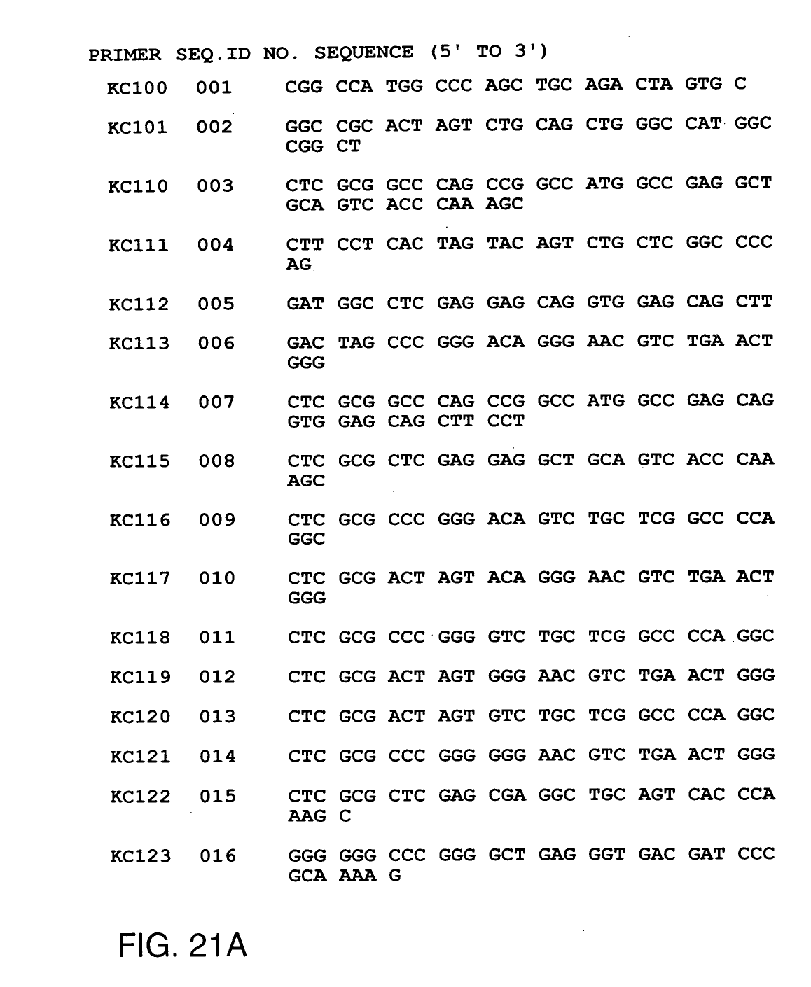

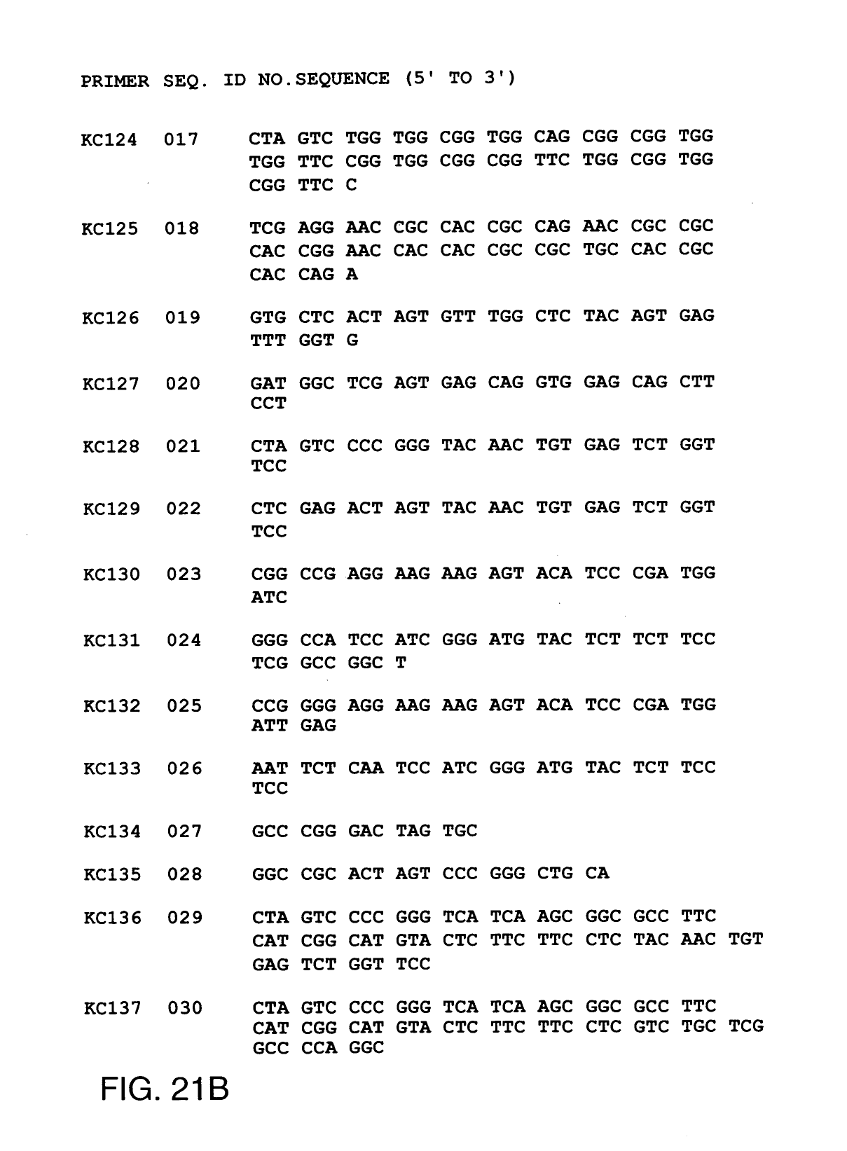

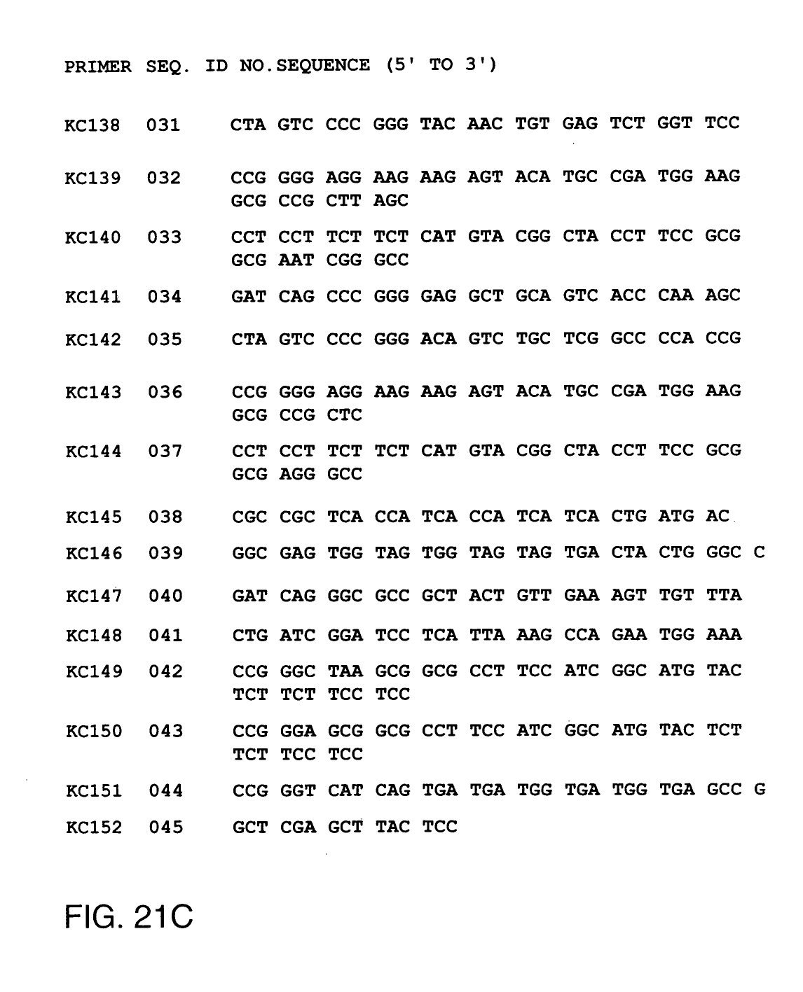

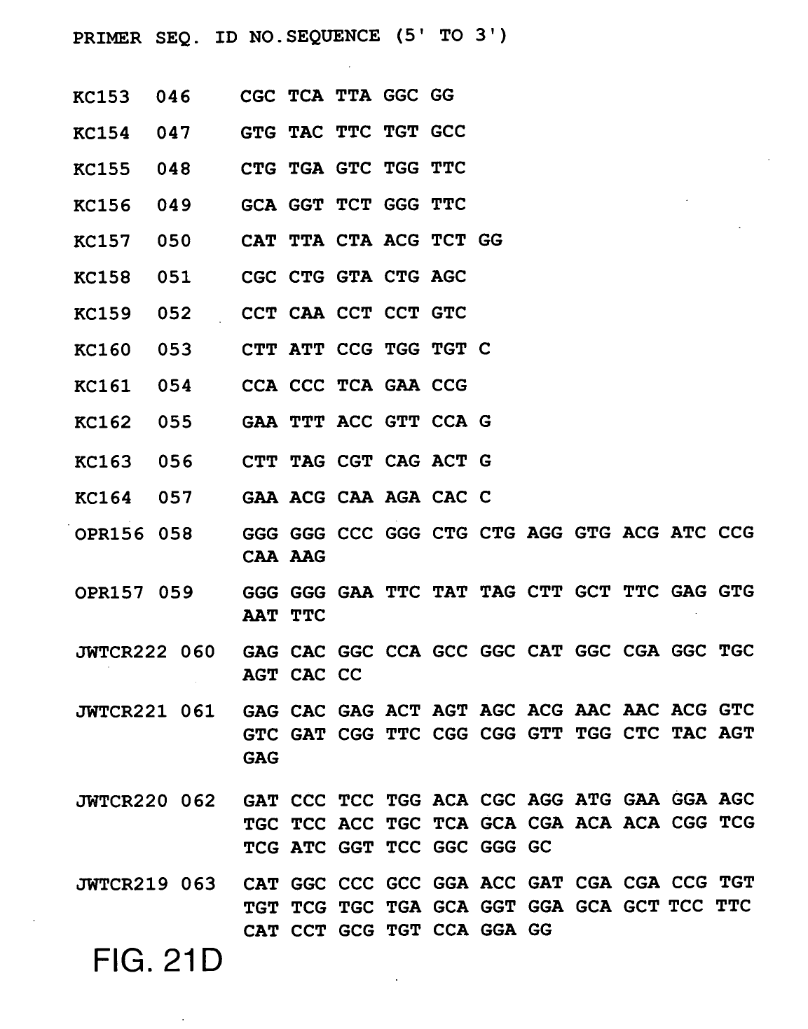

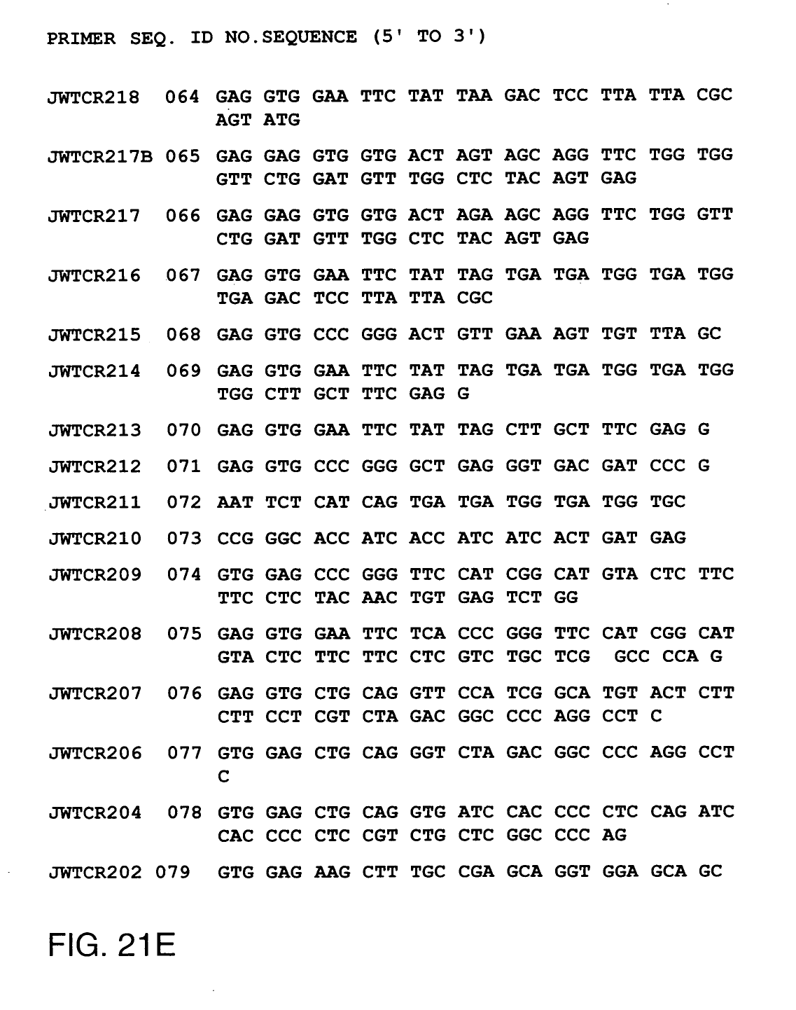

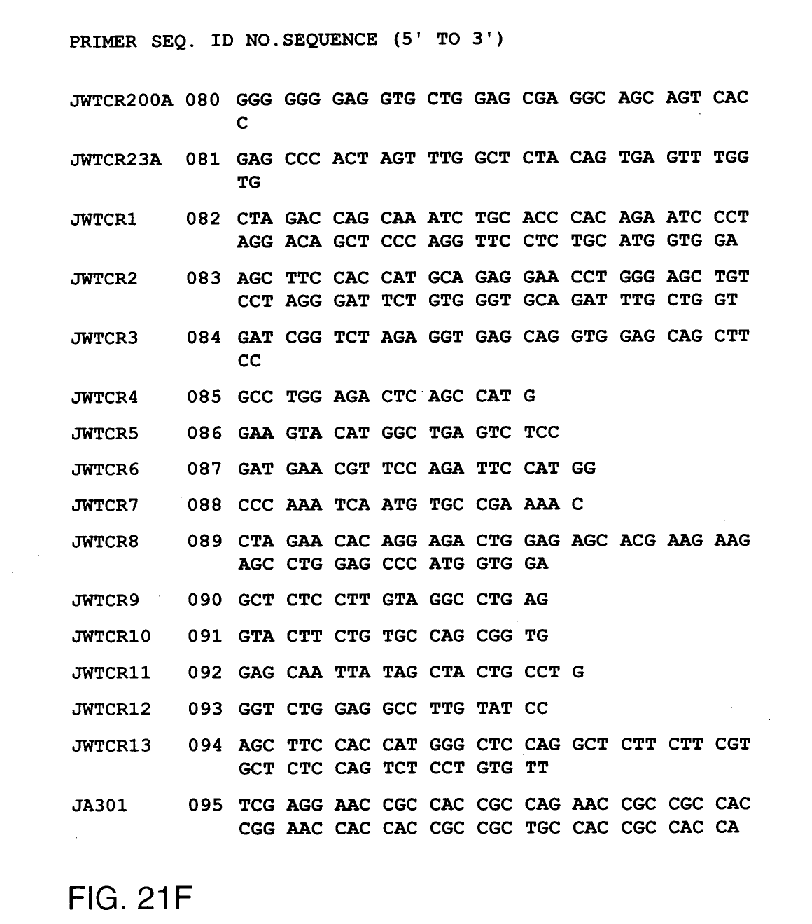

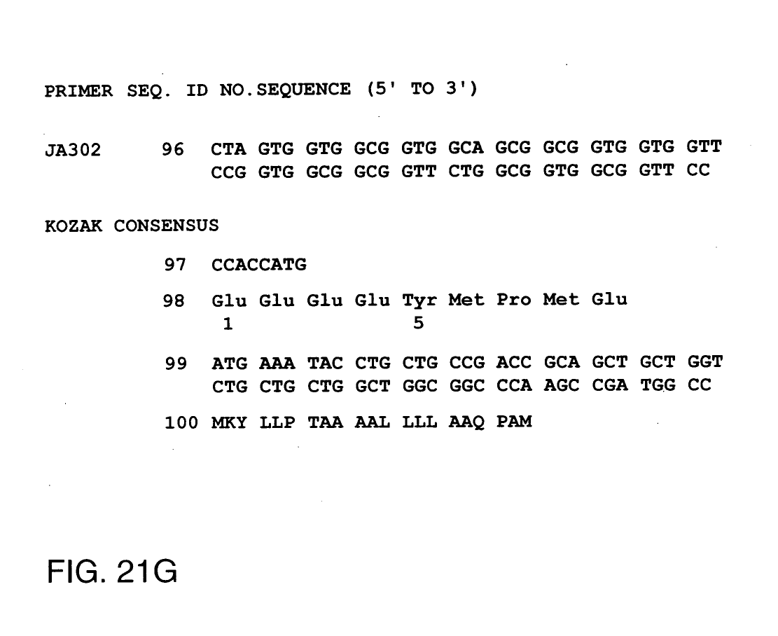

FIG. 20 schematically illustrates several recombinant bacteriophage vectors made using the f88-4 bacteriophage (pKC70, pKC71, and pKC72).

### EXAMPLE 18

### Construction of scTCR Bacteriophage Libraries from HIV-infected Cells

A bacteriophage library displaying scTCR fusion molecules derived from CTLs of HIV-infected patients can be made in accordance with the methods described herein. HIV-infected patients to be used are those who have been diagnosed or categorized as long-term non-progressors (ie. LTNP). These HIV-infected patients have been studied for CTL responses to various HIV antigens including gag and pol. CTL profiles from the patients typically show strong immunoactivity to HIV antigens in comparison to patients who are susceptible to AIDS. See generally Miedema, F. and Klein, M. R. Science (1996) 272, 505.

To identify TCRs participating in the CTL response and to construct corresponding fusion proteins, a class I HLA-A2 restricted human TCR library can be made from T cells isolated from the LTNP patients in accordance with the examples described above.

Several methods can be employed to prepare the class I HLA-A2 restricted bacteriophage library. For example, one way is to isolate and pool 10⁷ T cells from three HLA-A2 LTNP patients as described previously (see Altman, J. D. et al. Science (1996) 274:94). Messenger RNA can be purified from these cells and cDNA made by standard procedures such as by using an oligonucleotide primer to human C-α and C-β TCRs.

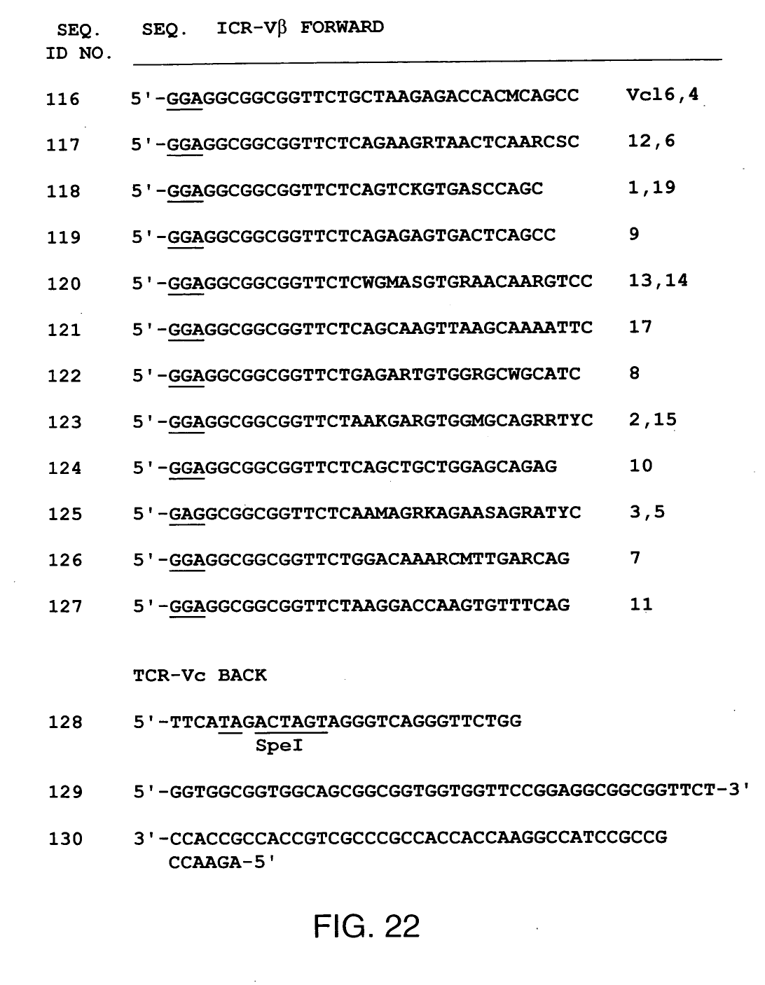

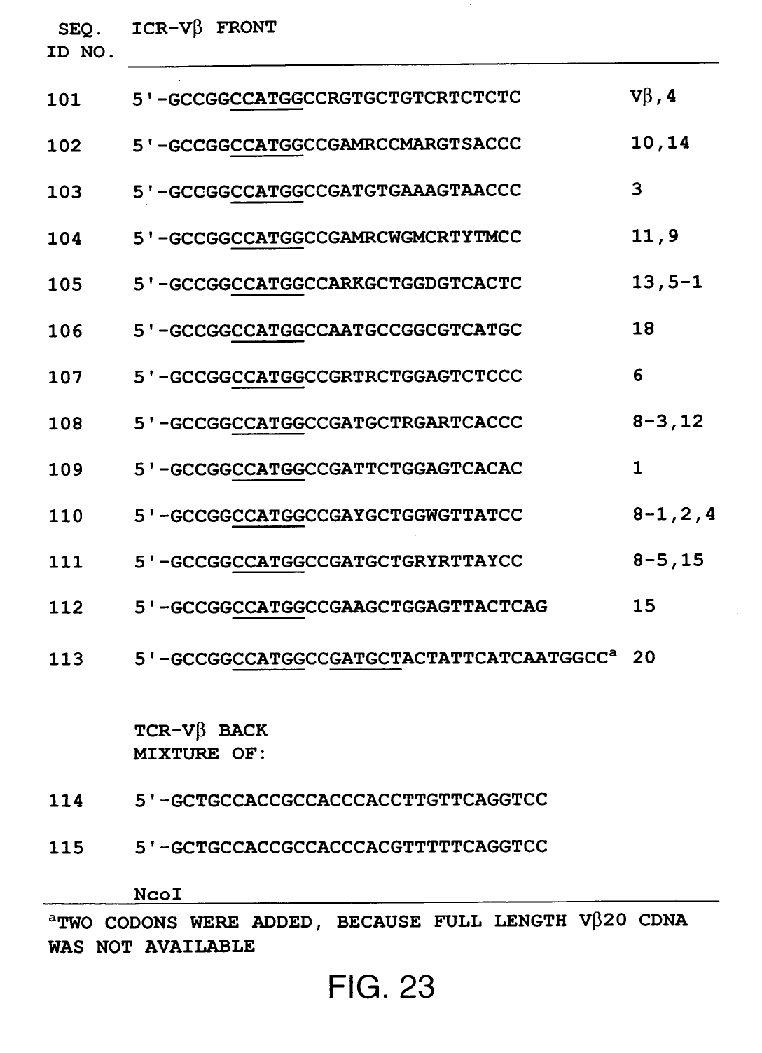

Exemplary primers are disclosed in FIG. 22 (SEQ ID NOs: 116 to 128) and FIG. 23 (SEQ ID NOs.: 101-115). More specifically, the V-α genes can be PCR-amplified using 12 forward primers (FIG. 22 SEQ ID NO. 116-127) and a single back primer (SEQ ID NO.: 128) that hybridizes at the 5′ end of the C-α gene sequence. The β chain can be PCR-amplified by using 13 forward primers (FIG. 23, SEQ ID NOs: 101-113) that hybridize at the 5′ end of V-β chain and two back primers (FIG. 23 SEQ ID NOs: 114, 115) situated 378 bases into the β constant domain. The PCR amplification can be performed in accordance with standard methods.

The PCR-amplified DNA can be inserted into suitable DNA vectors that a bacteriophage coat protein or fragment thereof such as those disclosed in Example 14. More particularly, the pKC45 DNA vector discribed in Example 2 can be used to subclone V-α chains into the SfiI and SpeI of the vector, and the V-β/C-β chains can be cloned into the XmaI site therein. A suitable vector encoding the scTCR bacteriophage fusion protein can then be made in accordance with the above examples. Alternatively, the V-α chains, and V-β/C-β chains can be subcloned into the f-88 bacteriophage vector (see Example 17), e.g., as an insertable HindIII to PSTI fragment. The recombinant bacteriophages thus produced can be used to infect suitable host cells and to propagate and screen recombinant bacteriophage in accordance with examples disclosed previously.

In some cases, it may be desirable to modify cloning sites of the f88 bacteriophage in accordance with standard recombinant techniques to permit cloning of the particular fragments (e.g., an SfiI-XmaI fragment) such as by including suitable linker sequences. The modification can be accomplished, e.g., by annealing suitable restriction site primers into HindIII and PstI sites of the bacteriophage. The bacteriophage display library thus produced can be amplified and stored in accordance with standard procedures.

### EXAMPLE 19

### Screening Bacteriophage Display Libraries from HIV-infected Cells

The recombinant bacteriophage library produced in Example 18 can be screened by several alternative approaches. For example, the library can be screened with a variety of detectably-labelled probes including whole (inactivated) HIV virus, HIV proteins, particularly HIV coat proteins, and APCs expressing HIV peptides in the context of an MHC/HLA complex. The screen can be conducted with a peptide epitope of an HIV coat protein which is known to stimulate an immune response against the HIV virus in vivo. Several examples of such peptide epitopes are known and include peptides isolated from the HIV gp120, gp41, gp160, gag and pol coat proteins.

a) Screening with HIV gp120 Coat Proteins

Peptide epitopes from the V3 loop of the gp120 protein can be used to screen the bacteriophage library. Exemplary peptides include the T1 (amino acids 428-443) and T2 (amino acids 112-124) peptides of the HIV gp120 protein. Peptide epitopes from the gp120 V3 loop are among those capable of inducing HIV neutralizing antibodies (see Berzofsky, J. A. et al. (1991) FASEB J. 5: 2412; Ahlers, J. D. et al, (1993) J. Immunology 150: 5647). See also Karzon, D. T. et al. and Hart, J. K. et al. for examples of other peptide epitopes which can be used to screen the bacteriophage library for scTCR fusion proteins (Karzon, D. T. et al. (1992) Vaccine 14: 1039; Hart, J. K et al. PNAS (USA) (1991) 88: 9448).

B. Biopanning with APCs Expressing HIV Coat Proteins

The scTCR library constructed in Example 18, above, can be panned for scTCR fusion proteins which bind immunogenic HIV peptides such as those derived from the HIV gag or pol protein. Thee HIV coat proteins are presented in the context of cells expressing single-chain HLA-A2 molecules. The single-chain HLA-A2 molecules are made as described in Garboczi, D. N. et al. PNAS (USA) (1992) 89:3429. Cells expressing the single chain HLA-A2 molecules can be made in accordance with known methods (Altman, J. D. et al., supra).

Biopanning can be accomplished by several methods. For example, one way is to use two different presentations of the MHC/peptide target. An initial round of panning will use transfected cells expressing HLA-2 and either gag or pol peptides as a target antigen. Panning is performed by using approximately 10¹¹ bacteriophage from the bacteriophage display library described in Example 18. The bacteriophage library is then contacted with cells presenting the HLA-A2 and gag or pol peptide. Bacteriophage can be incubated with approximately 10⁶ transfectants for two hours at 4° C., washed 2 times in 2% FBS/PBS and then eluted in 100 ul of 0.1M citrate buffer, pH 5.0 and neutralized with 10 ul of 0.1M Tris, pH 8.0. Eluted bacteriophage can be propagated by infecting a suitable host such as E. coli strain K91kan, and growing bacteriophage overnight. Bacteriophage particles can be purified by standard procedures.

Multiple rounds of panning against the cells or a solid support bearing a suitable MHC/antigen can increase the purity of bacteriophages bearing the scTCR fusion proteins identified in the cell panning experiment. For example, the bacteriophages isolated by the cell panning method can be purified by panning against a solid support (e.g., a microtitre dish) coated with single chain HLA-A2 and peptide. After a two hour incubation, non-specific bacteriophage can be removed by washing wells about 8× with 0.3 ml of PBS/0.2% Tween. Bound bacteriophage can be eluted in 0.1 ml of 0.1M HCl in glycine, pH 3.0 and neutralized in 10 ul of 2M Tris, pH 8.0. Additional panning can be performed by alternating between use of cells and immobilized MHC/HLA peptide molecules. After approximately 5 or 6 rounds of panning, a substantially pure preparation of bacteriophage displaying scTCR will result. Subsequently, DNA can be isolated from the bacteriophages to determine the DNA sequence of the α and β variable domains of the scTCR encoded by the bacteriophages. It is expected that a bias or enrichment in the frequency of α or β variable domain usage is indicative of a productive interaction between a scTCR in the bacteriophage display library and an MHC/peptide antigen.

The detectably-labelled probe will be used to enrich the bacteriophage library for a recombinant bacteriophage of interest. For example, when the probe is a peptide epitope from the gp120 V3 loop, recombinant bacteriophages can be isolated which specifically bind the peptide. Typically, several enrichment steps will be performed in which the number of steps will depend on several factors such as, e.g., representation of the desired recombinant bacteriophage in the library, or avidity of the detectably-labelled probe. DNA from the recombinant bacteriophage will be isolated by standard means. DNA encoding the scTCR fusion protein will then be sequenced in accordance with standard methods. DNA sequencing of several recombinant bacteriophage isolates will be indicative of specific binding between the detectably-labelled probe and the scTCR fusion protein encoded by the recombinant bacteriophage.

High frequency clones will then be expressed as soluble and fully functional scTCR fusion proteins in suitable vectors such as the pKC60 and pKC62 vectors described previously. The expressed fusion proteins can then be individually purified if desired by immunoaffinity chromatography using the previously described H57 monoclonal antibody in accordance with standard methods.

### EXAMPLE 20

### Characterization of scTCR Fusion Proteins Detected by Anti-HIV Panning

Several alternative methods can be used to evaluate specific binding between the recombinant bacteriophage detected above and HIV antigen.

A) BioCore Analysis

In cases where an HIV peptide was used to screen the bacteriophage library described above in Example 18, the peptide can be used to make a scMHC class I molecule in accordance with methods described in published PCT Application No. published PCT/US95/09816, as well as said pending U.S. patent application Ser. Nos. 08/382,454 and 08/596,387. Preferably, the peptide will be covalently linked to the scMHC class I molecule. The scMHC class I peptide complex can then be used to coat chips for BioCore Analysis as described below in Examples 7 and 20. More specifically, single chain HLA-A2 molecules bound with gag or pol antigen (see Altman, J. D. et al. supra) can be covalently coupled through amine reactive sites to the biosensor chip. The chip is then contacted with a sample including the scTCR fusion protein. Specific binding can be detected on the chip by described above. See also Seth, A. et al. Nature (1994) 369: 324; Matsui, K. et al. PNAS (USA) (1994) 91: 12862). It is expected that most scTCR fusion protein interactions will be in the range of 10⁻⁵ to 10⁻⁷M. Detection of specific binding between the scMHC class I peptide complex and the scTCR fusion protein would indicate presence of a specific scTCR-peptide binding complex.

By determining a binding coefficient for each of the isolated scTCR fusion proteins the effectiveness of the receptor for binding and triggering cell death can be readily predicted.

B) Binding to scTCR Fusion Protein Tetramers

Specific binding between scTCR fusion proteins and an HIV peptide can also be detected by assaying binding with scTCR fusion protein tetramers in accordance with examples described above (see Example 9). Tetrameric scTCR fusion protein can also be made by inserting DNA encoding the fusion in a suitable DNA vector which includes a Bir A-dependent biotinylation site (see Schatz, P. J. Biotechnology (1993) 11: 1138). The scTCR fusion proteins can be further modified by adding avidin-phycoerythrin in accordance with standard methods to produce the tetrameric scTCR fusion proteins. Transfected cells which display the HIV peptide can be prepared as described above and in accordance with methods described in published PCT Application No. published PCT/US95/09816, as well as said pending U.S. patent application Ser. Nos. 08/382,454 and 08/596,387. The transfected cells can be used to screen the library and can be stained with the labelled scTCR fusion protein tetramer to detect specific binding to the cells. Stronger binding by FACS analysis would be indicative of a specific interaction of the scTCR fusion protein with the MHC class I peptide complex.

The scTCR fusion proteins isolated in Example 19, above, can be tested for binding affinity and activity to determine if the scTCR fusion proteins specifically bind the MHC/peptide complex. Examples of such suitable assays are those described in the examples above.

C. FACS Analysis
FACS can be used to detect interactions between the scTCR fusion proteins and target cells discussed in Example 16, above. For example, the scTCR fusion protein can be biotinylated in accordance with standard methods and combined with strepavidin-phycoerythrin to form labelled sc-TCR tetramers. FACS can be used to qualitatively measure the interaction of the scTCR and a suitable target cell such as A20 cells and tumor cell lines. Alternatively, FACS can be conducted by coupling the scTCR fusion proteins of Example 16 to a suitable solid support (e.g., latex beads) to display multiple copies of the scTCR. A related method has been shown to produce tetrameric class I MHC/peptide molecules (see e.g., Altman, J. D. et al. supra).

D. I1-Expression in gD12 T hybridoma Cells
Activation of gD12 T hybridoma cells can be readily assayed by measuring IL-2 activity as disclosed previously in said pending U.S. patent application Ser. No. 08/596,387. This assay can be used to assess the function of the scTCR fusion proteins produced in Example 16. For example, gD12 T hybridoma cells can be transfected with DNA encoding a scTCR molecule produced in Example in accordance with methods disclosed previously. FACS staining can be conducted as disclosed in Example 16 to detect scTCR receptor expression on the cell surface. Receptor positive clones can be used, in the IL-2 activation assay described in said published PCT Application No. PCT/US95/09816, as well as said pending U.S. patent application Ser. Nos. 08/382,454 and 08/596,387. If the scTCR receptor is active (ie. it specifically binds the MHC/peptide target), I1-2 production from the gD12 cells can be readily detected and measured.

### EXAMPLE 21

### Isolation of Autoimmune Protein Fusions Binding Class II Restricted MHC/peptide Complexes

The present methods can be used to isolate scTCR fusion proteins which specifically bind autoimmune proteins and peptides. For example, T cells can be isolated in accordance with conventional methods from individuals that are DR-2+ and have been diagnosed with multiple sclerosis. TCR DNA can be obtained from the T cells and used to construct a scTCR fusion proteins in accordance with methods described in the examples above. The bacteriophage display library can be made and tested in accordance with Examples 14-15, above.

The bacteriophage display library can be made by isolating 10⁷ T cells from several DR-2+ patients with multiple sclerosis. T cell mRNA can be purified and cDNA made as described in Example 1, above. PCR amplification of the TCR V-α and V-β/C-β chains can be performed by using the same primer set as described in Example 1. The V-α and V-β/C-β chains can be cloned into cloning sites of suitable bacteriophage display vectors such the SfiI-SpeI and XhoI-XmaI sites, respectively, of the f88-4 bacteriophage vector DNA disclosed in Example 17, above. The library can be amplified and stored according to standard methods to pan specific antigenic targets.

The single-chain DR-2 molecules are made in general accordance with methods described above, in said published PCT Application No. PCT/US95/09816, said pending U.S. patent application Ser. Nos. 08/382,454 and 08/596,387, as well as Rhode, P. et al. J. of Mol. Immunology, (1996) 32, 555. More specifically, HLA-DR2 molecules can be purified from EBV transformed lymphoblastoid cells which present the HLA-DR2 molecule. Briefly, the HLA-DR2 molecule can be purified by standard immunoaffinity chromatography by solubilizing the cells with buffer including Triton X-100. The cell lysate is then applied to an antibody-Sepharose column. DR2 is bound and eluted in phosphate buffer containing 0.05% N-dodecyl B-D-maltoside detergent at pH 11.3. Fractions are immediately neutralized with 1M acetic acid and the DR2 pool is collected through a DEAE ion exchange column in phosphate containing 0.5M NaCl pH 8.0. The protein fraction can be assayed for purity by SDS-PAGE gel electrophoresis followed by silver staining.

The bacteriophage library can be screened by several methods, particularly panning against a solid support coated with the single-chain DR-2 molecule in accordance with methods described previously. The panning can be conducted by producing single-chain DR-2 molecules in a host such as insect cells and immobilizing the single-chain DR-2 molecules on a solid support such as walls of a microtitre plate.

Additionally, the bacteriophage library can be purified with cells that have been transfected with DR-2 molecules (see DeKruif, J. et al. PNAS (USA) (1995) 92, 3938. Enrichment for bacteriophage particles expressing scTCR fusion proteins which specifically bind the DR-2 protein can be performed as described above in the above examples.

The invention has been described with reference to preferred embodiments thereof. However, it will be appreciated that those skilled in the art, upon consideration of this disclosure, may make modifications and improvements within the spirit and scope of the invention.

1. A soluble fusion protein comprising a bacteriophage coat protein covalently linked to a single-chain T cell receptor, wherein the single-chain T cell receptor comprises a V-α chain covalently linked to a V-β chain by a peptide linker sequence.

2. The soluble fusion protein of claim 1, wherein the C-terminus of the V-α chain is covalently linked by the peptide linker sequence to the N-terminus of V-β chain.

3. The soluble fusion protein of claim 1, wherein the C-terminus of the V-β chain is covalently linked by the peptide linker sequence to the N-terminus of the V-α chain.

4. The soluble fusion protein of claim 2 further comprising a C-β chain fragment covalently linked between the C-terminus of the V-β chain and the N-terminus of the bacteriophage coat protein.

5. The soluble fusion protein of claim 2 further comprising a C-α chain fragment covalently linked between the C-terminus of the V-α chain and the N-terminus of the peptide linker sequence.

6. The soluble fusion protein of claim 2, wherein the fusion protein further comprises at least one protein tag.

7. The soluble fusion protein of claim 2, wherein the peptide linker sequence contains from approximately 2 to 20 amino acids.

8. The soluble fusion protein of claim 1, wherein the bacteriophage coat protein is gene III or gene VIII protein.

9. A soluble fusion protein comprising covalently linked in sequence: 1) a V-α chain, 2) a peptide linker sequence, 3) a V-β chain and 3) a bacteriophage gene III protein.

10. The soluble fusion protein of claim 9 further comprising a C-β chain fragment covalently linked between the C-terminus of the V-β chain and the N-terminus of the bacteriophage gene III protein.

11. The soluble fusion protein of claim 10, further comprising a protein tag covalently linked to the C-terminus of the C-β fragment and the N-terminus of the bacteriophage gene III protein.

12. The soluble fusion protein of claim 9 further comprising a first protein tag covalently linked between the C-terminus of the V-β chain and the N-terminus of the bacteriophage gene III protein, and a second protein tag covalently linked to the C-terminus of the fusion protein.

13.-17. (canceled)

18. The soluble fusion protein of claim 2, wherein the V-α and V-β chains are isolated from cytotoxic T cells.

19. (canceled)

20. The single-chain T cell receptor of claim 6, wherein the single-chain T cell receptor has been humanized.

21. A DNA segment comprising a sequence encoding a soluble fusion protein, the soluble fusion protein comprising a bacteriophage coat protein covalently linked to a single-chain T cell receptor, wherein the DNA segment further comprises an operably linked promoter and linker sequence.

22. A DNA segment comprising a sequence encoding a soluble fusion protein comprising covalently linked in sequence: 1) a V-α chain, 2) a peptide linker sequence, 3) a V-β chain and 3) a bacteriophage gene III protein.

23. A DNA segment comprising a sequence encoding a soluble fusion protein comprising covalently linked in sequence: 1) a V-α chain, 2) a peptide linker sequence, 3) a V-P chain, and 4) a bacteriophage gene VIII protein.

24. The DNA segment of claim 22, wherein the V-β chain is covalently linked to a C-β chain fragment.

25. The DNA segment of claim 22, wherein the V-α chain is covalently linked to a C-α chain fragment.

26. The DNA segment of claim 23 further comprising a sequence encoding a protein tag covalently linked between the 3′ end of the sequence encoding the V-β chain and the 5′ end of the sequence encoding the bacteriophage gene VIII protein.

27. The DNA segment of claim 24 or 25 further comprising a sequence encoding a protein tag covalently linked between the 3′ end of the sequence encoding the C-β chain fragment and the 5′ end of the sequence encoding the bacteriophage gene VIII protein.

28. The DNA segment of claim 26 further comprising sequence encoding a protein tag covalently linked to the 3′ end of the sequence encoding the fusion protein.

29. A DNA vector comprising the DNA segment of claim 21.

30. The DNA segment of claim 21, wherein the promoter and linker are phoA and pelB from E. coli, respectively.

31. A bacteriophage library comprising bacteriophages displaying soluble fusion proteins, wherein each of the soluble fusion proteins comprises a bacteriophage coat protein covalently linked to a single-chain T cell receptor, wherein each single-chain T cell receptor comprises a V-α chain covalently linked to a V-β chain by a peptide linker sequence.

32-42. (canceled)

43. A kit comprising the bacteriophage display library of claim 31, a host cell sample, and directions for using the kit.

44.-50. (canceled)

51. A method of increasing the specific binding affinity of a single-chain T cell receptor for a ligand, the method comprising:
determining a first specific binding affinity between the single-chain T cell receptor and the ligand,
infecting host cells with the bacteriophage library of claim 44, the infecting being under conditions which permit propagation of the bacteriophages,
contacting the host cells with the ligand sufficient to permit specific binding between at least one of the bacteriophages and the ligand to produce at least one specific binding complex between the bacteriophage and the ligand,
identifying one of the bacteriophages comprising the specific binding complex,
isolating DNA from the bacteriophage, the DNA comprising a sequence encoding a soluble fusion protein mutein and expressing the soluble fusion protein mutein,
separating a soluble single-chain T cell receptor mutein from the soluble fusion protein mutein,
determining a second specific binding affinity between the single-chain T cell receptor mutein and the ligand; and
identifying the single-chain T cell receptor with increased specific binding affinity for the ligand as the single-chain T cell receptor mutein in which the second specific binding affinity is greater than the first specific binding affinity.

52.-53. (canceled)

54. A method of inducing an immune response in a mammal comprising administering to the mammal an effective amount of a single-chain T cell receptor cleaved from a soluble fusion protein comprising a bacteriophage coat protein covalently linked to the single-chain T cell receptor, wherein the immune response is capable of immunizing the mammal against T cell receptor epitopes on the surfaces of pathogenic T cells.

55.-57. (canceled)

58. A method of detecting a molecule capable of inhibiting specific binding between a ligand and a T cell receptor, the method comprising:
incubating a soluble fusion protein comprising a bacteriophage coat protein covalently linked to a single-chain T cell receptor, the incubating being in the presence of the ligand,
incubating a soluble fusion protein comprising a bacteriophage coat protein covalently linked to a single-chain T cell receptor, the incubating being in the presence of the ligand and the molecule; and
evaluating the interaction between the ligand and the soluble fusion protein in the absence and presence of the molecule,
wherein less interaction between the fusion protein and the ligand in the presence of the molecule than in the absence of the molecule is indicative of the molecule capable of inhibiting specific binding between the ligand and the T cell receptor.

59. (canceled)
